SEQUENCE LISTING (7 sequences)

SEQ ID NO: 1 - protein, 5 aa, Unknown
  source: /note=“Description of Unknown PD-1 motif peptide”
MYPPY

SEQ ID NO: 2 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 118 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 7 - protein, 110 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

(19) United States
(12) Patent Application Publication
(10) Pub. No.: US 2017/0340733 A1
(43) Pub. Date: Nov. 30, 2017

(54) COMBINATION THERAPIES

(71) Applicants: Zhu Alexander CAO, Cambridge, MA (US); NOVARTIS AG, Basel (CH)

(72) Inventor: Zhu Alexander Cao, Lawrenceville, NJ (US)

(73) Assignee: NOVARTIS AG, Basel (CH)

(21) Appl. No.: 15/536,718

(22) PCT Filed: Dec. 18, 2015
(86) PCT No.: PCT/US2015/066812

§ 371 Date: Jun. 16, 2017

Related U.S. Application Data
(60) Provisional application No. 62/094,901, filed on Dec. 19, 2014.

(51) Int. Cl. A61K 39/395; A61K 31/4184; A61K 31/4196; A61K 31/4365; A61K 31/4439; C07K 16/30; C07K 16/28; C07K 16/24; A61K 38/13; A61K 38/12; A61K 47/68; A61K 31/551; A61K 31/5377; A61K 31/519; A61K 31/517; A61K 31/506; A61K 31/502; A61K 31/501; A61K 31/497; A61K 31/496; A61K 31/4745; A61K 31/4709; A61K 31/553; A61K 39/00

(52) U.S. Cl. CPC A61K 39/3955; A61K 31/553; A61K 31/4184; A61K 31/4196; A61K 31/4365; A61K 31/4439; C07K 16/3061; C07K 16/30; C07K 16/2869; C07K 16/2827; C07K 16/2818; C07K 16/2803; C07K 16/243; A61K 39/39558; A61K 38/13; A61K 38/12; A61K 47/6803; A61K 31/551; A61K 31/5377; A61K 31/519; A61K 31/517; A61K 31/506; A61K 31/502; A61K 31/501; A61K 31/497; A61K 31/496; A61K 31/4745; A61K 31/4709; A61K 2039/545; C07K 2317/51; C07K 2317/515; C07K 2317/55; C07K 2317/56

(57) ABSTRACT

Combination therapies are disclosed. The combination therapies can be used to treat or prevent cancerous conditions and/or disorders.

### CROSS REFERENCE TO RELATED APPLICATIONS

This application claims the benefit of U.S. Provisional Application No. 62/094,901, filed Dec. 19, 2014, the contents of the aforementioned application are hereby incorporated by reference in their entirety.

### SEQUENCE LISTING

This instant application contains a Sequence Listing which has been submitted electronically in ASCII format and is hereby incorporated by reference in its entirety. Said ASCII copy, created on Dec. 18, 2015, is named C2160-7007WO_SL.txt and is 14,618 bytes in size.

### BACKGROUND

The ability of T cells to mediate an immune response against an antigen requires two distinct signaling interactions (Viglietta, V. et al. (2007) Neurotherapeutics 4:666-675; Korman, A. J. et al. (2007) Adv. Immunol. 90:297-339). First, an antigen that has been arrayed on the surface of antigen-presenting cells (APC) is presented to an antigen-specific naive CD4⁺ T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The immune system is tightly controlled by a network of costimulatory and co-inhibitory ligands and receptors. These molecules provide the second signal for T cell activation and provide a balanced network of positive and negative signals to maximize immune responses against infection, while limiting immunity to self (Wang, L. et al. (Epub Mar. 7, 2011) J. Exp. Med. 208(3):577-92; Lepenies, B. et al. (2008) Endocrine, Metabolic & Immune Disorders—Drug Targets 8:279-288). Examples of costimulatory signals include the binding between the B7.1 (CD80) and B7.2 (CD86) ligands of the APC and the CD28 and CTLA-4 receptors of the CD4⁺ T-lymphocyte (Sharpe, A. H. et al. (2002) Nature Rev. Immunol. 2:116-126; Lindley, P. S. et al. (2009) Immunol. Rev. 229:307-321). Binding of B7.1 or B7.2 to CD28 stimulates T cell activation, whereas binding of B7.1 or B7.2 to CTLA-4 inhibits such activation (Dong, C. et al. (2003) Immunolog. Res. 28(1):39-48; Greenwald, R. J. et al. (2005) Ann. Rev. Immunol. 23:515-548). CD28 is constitutively expressed on the surface of T cells (Gross, J., et al. (1992) J. Immunol. 149:380-388), whereas CTLA4 expression is rapidly up-regulated following T-cell activation (Linsley, P. et al. (1996) Immunity 4:535-543).

Other ligands of the CD28 receptor include a group of related B7 molecules, also known as the “B7 Superfamily” (Coyle, A. J. et al. (2001) Nature Immunol. 2(3):203-209; Sharpe, A. H. et al. (2002) Nature Rev. Immunol. 2:116-126; Collins, M. et al. (2005) Genome Biol. 6:223.1-223.7; Korman, A. J. et al. (2007) Adv. Immunol. 90:297-339). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins, M. et al. (2005) Genome Biol. 6:223.1-223.7).

The Programmed Death 1 (PD-1) protein is an inhibitory member of the extended CD28/CTLA4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72). Although structurally similar to CTLA-4, PD-1 lacks the MYPPY motif (SEQ ID NO: 1) that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Given the importance of immune checkpoint pathways in regulating an immune response, the need exists for developing novel combination therapies that activate the immune system.

### SUMMARY

The present invention provides, at least in part, methods and compositions comprising an immunomodulator (e.g., one or more of: an activator of a costimulatory molecule or an inhibitor of an immune checkpoint molecule) in combination with a second therapeutic agent chosen from one or more of the agents listed in Table 1. In one embodiment, an inhibitor of an immune checkpoint molecule (e.g., one or more inhibitors of PD-1, PD-L1, LAG-3, TIM-3, CEACAM (e.g., CEACAM-1, -3 and/or -5), or CTLA-4) can be combined with a second therapeutic agent chosen from one or more agents listed in Table 1 (e.g., one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor). The combinations described herein can provide a beneficial effect, e.g., in the treatment of a cancer, such as an enhanced anti-cancer effect, reduced toxicity and/or reduced side effects. For example, the immunomodulator, the second therapeutic agent, or both, can be administered at a lower dosage than would be required to achieve the same therapeutic effect compared to a monotherapy dose. Thus, compositions and methods for treating hyperproliferative disorders including cancer using the aforesaid combination therapies are disclosed.

Accordingly, in one aspect the invention features a method of treating (e.g., inhibiting, reducing, ameliorating, or preventing) a disorder, e.g., a hyperproliferative condition or disorder (e.g., a cancer) in a subject. The method includes administering to the subject an immunomodulator (e.g., one or more of: an activator of a costimulatory molecule or an inhibitor of an immune checkpoint molecule) and a second therapeutic agent, e.g., a second therapeutic agent chosen from one or more of the agents listed in Table 1, thereby treating the disorder, e.g., the hyperproliferative condition or disorder (e.g., the cancer). In certain embodiments, the immunomodulator is an inhibitor of an immune checkpoint molecule (e.g., an inhibitor of PD-1, PD-L1, LAG-3, TIM-3, CEACAM (e.g., CEACAM-1, -3 and/or -5), or CTLA-4, or any combination thereof). In other embodiments, the second therapeutic agent is chosen from one or more of the agents listed in Table 1, or disclosed in a publication listed in Table 1. In some embodiments, the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor.

The combination of the immunomodulator and the second agent can be administered together in a single composition or administered separately in two or more different compositions, e.g., compositions or dosage forms as described herein. The administration of the immunomodulator and the second agent can be in any order. For example, the immunomodulator can be administered concurrently with, prior to, or subsequent to, the second agent. When administered in combination, the immunomodualtor, the additional agent (e.g., second or third agent), or all, can be administered in an amount or dose that is higher, lower or the same than the amount or dosage of each agent used individually, e.g., as a monotherapy. In certain embodiments, the administered amount or dosage of the immunomodulator, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the immunomodulator, the additional agent (e.g., second or third agent), or all, that results in a desired effect (e.g., treatment of cancer) is lower (e.g., at least 20%, at least 30%, at least 40%, or at least 50% lower).

In another aspect, the invention features a method of reducing an activity (e.g., growth, survival, or viability, or all), of a hyperproliferative (e.g., a cancer) cell. The method includes contacting the cell with an immunomodulator (e.g., one or more of: an activator of a costimulatory molecule or an inhibitor of an immune checkpoint molecule) and a second therapeutic agent, e.g., a second therapeutic agent chosen from one or more of the agents listed in Table 1, thereby reducing an activity in the cell. In certain embodiments, the immunomodulator is an inhibitor of an immune checkpoint molecule (e.g., an inhibitor of PD-1, PD-L1, LAG-3, TIM-3, CEACAM (e.g., CEACAM-1, -3 and/or -5), or CTLA-4, or any combination thereof). In other embodiments, the second therapeutic agent is chosen from one or more of the agents listed in Table 1, or disclosed in a publication listed in Table 1. In some embodiments, the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) a transduction modulator and/or apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor.

In some embodiments, the methods described herein can be used in vitro. For example, in vitro hPBMC-based assays can be used to screen for combination signals of immunomodulators and second therapeutic agents, as disclosed, e.g., in Wang, C. et al. (2014) Cancer Immunology Research 2:846-856. In some embodiments, the methods described herein can be used in vivo, e.g., in an animal subject or model or as part of a therapeutic protocol. The contacting of the cell with the immunomodulator and the second agent can be in any order. In certain embodiments, the cell is contacted with the immunomodulator concurrently, prior to, or subsequent to, the second agent. In some embodiments, the method described herein is used to measure tumor lymphocyte infiltration (TLI) in vitro or in vivo, as disclosed, e.g., in Frederick, D. T. et al. (2013) Clinical Cancer Research 19:1225-31.

In some embodiments, the method includes contacting the cell with an immunomodulator (e.g., one or more of: an activator of a costimulatory molecule or an inhibitor of an immune checkpoint molecule) and/or a second therapeutic agent, e.g., a second therapeutic agent chosen from one or more of the agents listed in Table 1, or disclosed in a publication listed in Table 1, in an animal model. In some embodiments, the animal model has a mutation that inhibits or activates a target described herein, e.g., PKC, HSP90, cKIT, ALK, CDK4/6, PI3K, mTOR, BRAF, FGF receptor, IGF-1R, and/or VEGFR. In one exemplary embodiment, an animal model is a mouse model with an inactivated p110δ isoform of PI3 kinase (e.g., p110δ^{D910A}) as disclosed, e.g., in Ali K., et al., (2014) Nature 510:407-411.

In some embodiments, an immune phenotype is determined by measuring one or more of expression, activation, signalling, flow cytometry, mRNA analysis, cytokine levels and/or immunohistochemisty. In some embodiments, the immune phenotype is determined systemically, e.g., in PBMCs. In some embodiments, the immune phenotype is determined in situ, e.g, in tumor cells.

In some embodiments, one or more of the following parameters is characterized to determine an immune phenotype: checkpoint induction; level of M1 macrophages relative to level of M2 macrophages; level of effector T cells relative to level of regulatory T cells; and/or level of T_{H1} cells relative to T_{H2/H17} cells.

In another aspect, the invention features a composition (e.g., one or more compositions or dosage forms), comprising an immunomodulator (e.g., one or more of: an activator of a costimulatory molecule or an inhibitor of an immune checkpoint molecule) and a second therapeutic agent, e.g., a second therapeutic agent chosen from one or more of the agents listed in Table 1, or disclosed in a publication listed in Table 1. In certain embodiments, the immunomodulator is an inhibitor of an immune checkpoint molecule (e.g., an inhibitor of PD-1, PD-L1, LAG-3, TIM-3, CEACAM (e.g., CEACAM-1, -3 and/or -5), or CTLA-4, or any combination thereof). In other embodiments, the second therapeutic agent is chosen from one or more of the agents listed in Table 1, or disclosed in a publication listed in Table 1. In some embodiments, the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) a transduction modulator and/or apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor. In one embodiment, the composition comprises a pharmaceutically acceptable carrier. The immunomodulator and the second agent can be present in a single composition or as two or more different compositions. The immunomodulator and the second agent can be administered via the same administration route or via different administration routes. In one embodiment, the pharmaceutical composition comprises the immunomodulator and the second agent separately or together.

Formulations, e.g., dosage formulations, and kits, e.g., therapeutic kits, that include the immunomodulator (e.g., one or more of: an activator of a costimulatory molecule or an inhibitor of an immune checkpoint molecule) and the second therapeutic agent, e.g., a second therapeutic agent chosen from one or more of the agents listed in Table 1, or disclosed in a publication listed in Table 1, and instructions for use, are also disclosed.

In one embodiment, the composition, formulation or combination is for use as a medicine, e.g., for the treatment of a proliferative disease (e.g., a cancer as described herein). In some embodiments, the immunomodulator and the second agent are administered concurrently, e.g., independently at the same time or within an overlapping time interval, or separately within time intervals. In certain embodiment, the time interval allows the immunomodulator and the second agent to be jointly active. In one embodiment, the composition, formulation or combination includes an amount which is jointly therapeutically effective for the treatment of a proliferative disease, e.g., a cancer as described herein.

In another aspect, the invention features a use of a composition (e.g., one or more compositions, formulations or dosage formulations) or a combination, comprising an immunomodulator (e.g., one or more of: an activator of a costimulatory molecule or an inhibitor of an immune checkpoint molecule) and a second therapeutic agent, e.g., a second therapeutic agent chosen from one or more of the agents listed in Table 1, or disclosed in a publication listed in Table 1, for the manufacture of a medicament for treating a proliferative disease, e.g., a cancer. In certain embodiments, the immunomodulator is an inhibitor of an immune checkpoint molecule (e.g., an inhibitor of PD-1, PD-L1, LAG-3, TIM-3, CEACAM (e.g., CEACAM-1, -3 and/or -5) or CTLA-4, or any combination thereof). In other embodiments, the second therapeutic agent is chosen from one or more of the agents listed in Table 1, e.g., one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) a transduction modulator and/or apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor.

Additional features or embodiments of the methods, compositions, dosage formulations, and kits described herein include one or more of the following:

In certain embodiments, the immunomodulator is an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, or CD83 ligand.

In certain embodiments, the immunomodulator is an inhibitor of an immune checkpoint molecule. In one embodiment, the immunomodulator is an inhibitor of PD-1, PD-L1, PD-L2, CTLA-4, TIM-3, LAG-3, CEACAM (e.g., CEACAM-1, -3 and/or -5), VISTA, BTLA, TIGIT, LAIR1, CD160, 2B4 and/or TGFR beta. In one embodiment, the inhibitor of an immune checkpoint molecule inhibits PD-1, PD-L1, LAG-3, TIM-3, CEACAM (e.g., CEACAM-1, -3 and/or -5), CTLA-4, or any combination thereof.

Inhibition of an inhibitory molecule can be performed at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig). In other embodiments, the inhibitor of the inhibitory signal is an antibody or antigen-binding fragment thereof, that binds to the inhibitory molecule; e.g., an antibody or fragment thereof (also referred to herein as “an antibody molecule”) that binds to PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of: Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specifity, e.g., a second binding specificity to TIM-3, CEACAM (e.g., CEACAM-1, -3 and/or -5), LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 or PD-L1 and CEACAM (e.g., CEACAM-1, -3 and/or -5). In another embodiment, the bispecific antibody molecule binds to PD-1 or PD-L1 and CEACAM-1. In still another embodiment, the bispecific antibody molecule binds to PD-1 or PD-L1 and CEACAM-3. In yet another embodiment, the bispecific antibody molecule binds to PD-1 or PD-L1 and CEACAM-5. In another embodiment, the bispecific antibody molecule binds to PD-1 or PD-L1. In yet another embodiment, the bispecific antibody molecule binds to PD-1 and PD-L2. In another embodiment, the bispecific antibody molecule binds to TIM-3 and LAG-3. In another embodiment, the bispecific antibody molecule binds to CEACAM (e.g., CEACAM-1, -3 and/or -5) and LAG-3. In another embodiment, the bispecific antibody molecule binds to CEACAM (e.g., CEACAM-1, -3 and/or -5) and TIM-3. Any combination of the aforesaid molecules can be made in a multispecific antibody molecule, e.g., a trispecific antibody that includes a first binding specificity to PD-1 or PD-1, and a second and third binding specifities to two or more of: TIM-3, CEACAM (e.g., CEACAM-1, -3 and/or -5), LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3, CEACAM (e.g., CEACAM-1, -3 and/or -5) or CTLA-4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In another embodiment, the inhibitor of PD-1 or PD-L1, e.g., the anti-PD-1 or PD-L1 antibody molecule, is administered in combination with a CEACAM inhibitor (e.g., CEACAM-1, -3 and/or -5 inhibitor), e.g., an anti-CEACAM antibody molecule. In another embodiment, the inhibitor of PD-1 or PD-L1, e.g., the anti-PD-1 or PD-L1 antibody molecule, is administered in combination with a CEACAM-1 inhibitor, e.g., an anti-CEACAM-1 antibody molecule. In another embodiment, the inhibitor of PD-1 or PD-L1, e.g., the anti-PD-1 or PD-L1 antibody molecule, is administered in combination with a CEACAM-5 inhibitor, e.g., an anti-CEACAM-5 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. Other combinations of immunomodulators with a PD-1 inhibitor (e.g., one or more of PD-L2, CTLA-4, TIM-3, LAG-3, CEACAM (e.g., CEACAM-1, -3 and/or -5), VISTA, BTLA, TIGIT, LAIR1, CD160, 2B4 and/or TGFR) are also within the present invention. Any of the antibody molecules known in the art or disclosed herein can be used in the aforesaid combinations of inhibitors of checkpoint molecule.

In other embodiments, the immunomodulator is an inhibitor of CEACAM (e.g., CEACAM-1, -3 and/or -5), e.g., human CEACAM (e.g., CEACAM-1, -3 and/or -5). In one embodiment, the immunomodulator is an inhibitor of CEACAM-1, e.g., human CEACAM-1. In another embodiment, the immunomodulator is an inhibitor of CEACAM-3, e.g., human CEACAM-3. In another embodiment, the immunomodulator is an inhibitor of CEACAM-5, e.g., human CEACAM-5. In one embodiment, the inhibitor of CEACAM (e.g., CEACAM-1, -3 and/or -5) is an antibody molecule to CEACAM (e.g., CEACAM-1, -3 and/or -5). The CEACAM (e.g., CEACAM-1, -3 and/or -5) inhibitor can be administered alone, or in combination with other immunomodulators, e.g., in combination with an inhibitor of LAG-3, TIM-3, PD-1, PD-L1 or CTLA-4.

In other embodiments, the immunomodulator is an inhibitor of LAG-3, e.g., human LAG-3. In one embodiment, the inhibitor of LAG-3 is an antibody molecule to LAG-3. The LAG-3 inhibitor can be administered alone, or in combination with other immunomodulators, e.g., in combination with an inhibitor of CEACAM (e.g., CEACAM-1, -3 and/or -5), TIM-3, PD-1, PD-L1 or CTLA-4.

In other embodiments, the immunomodulator is an inhibitor of TIM-3, e.g., human TIM-3. In one embodiment, the inhibitor of TIM-3 is an antibody molecule to TIM-3. The TIM-3 inhibitor can be administered alone, or in combination with other immunomodulators, e.g., in combination with an inhibitor of CEACAM (e.g., CEACAM-1, -3 and/or -5), LAG-3, PD-1, PD-L1 or CTLA-4.

### Exemplary Inhibitors of Immune Checkpoint Molecules

In one embodiment, the PD-1 inhibitor is an anti-PD-1 antibody chosen from Nivolumab, Pembrolizumab or Pidilizumab.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, WO2009/114335, and U.S. Pat. No. 8,354,509.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611. Other anti-PD1 antibodies are disclosed in U.S. Pat. No. 8,609,089, US 2010028330, and/or US 20120114649. Other anti-PD1 antibodies include AMP 514 (Amplimmune).

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224.

In some embodiments, the PD-L1 inhibitor is anti-PD-L1 antibody. In some embodiments, the anti-PD-L1 inhibitor is chosen from YW243.55.S70, MPDL3280A, MEDI-4736, MSB-0010718C, or MDX-1105.

In one embodiment, the PD-L1 inhibitor is MDX-1105. MDX-1105, also known as BMS-936559, is an anti-PD-L1 antibody described in WO2007/005874.

In one embodiment, the PD-L1 inhibitor is YW243.55.S70. The YW243.55.S70 antibody is an anti-PD-L1 described in WO 2010/077634 (heavy and light chain variable region sequences shown in SEQ ID Nos. 20 and 21, respectively).

In one embodiment, the PD-L1 inhibitor is MDPL3280A (Genentech/Roche). MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in WO2010/027827 and WO2011/066342).

In one embodiment, the LAG-3 inhibitor is an anti-LAG-3 antibody molecule. In one embodiment, the LAG-3 inhibitor is BMS-986016, disclosed in more detail herein below.

In another embodiment, the inhibitor of CEACAM (e.g., CEACAM-1, -3 and/or -5) is an anti-CEACAM antibody molecule. In one embodiment, the inhibitor of CEACAM is an anti-CEACAM-1 antibody as described in WO 2010/125571, WO 2013/82366 and WO 2014/022332, e.g., a monoclonal antibody 34B1, 26H7, and 5F4 or a recombinant form thereof, as described in, e.g., US 2004/0047858, U.S. Pat. No. 7,132,255 and WO 99/52552. In other embodiments, the anti-CEACAM antibody is an anti-CEACAM-1 and/or anti-CEACAM-5 antibody molecule as described in, e.g., WO 2010/125571, WO 2013/054331 and US 2014/0271618.

One or more of the aforesaid inhibitors of immune checkpoint molecules can be used in combination with one or more of the second agents disclosed in Table 1, or disclosed in a publication listed in Table 1, as more specifically exemplified below. In some embodiments, the second agent is chosen from one or more of:
1) 3-(1H-indol-3-yl)-4-[2-(4-methyl-1-piperazinyl)-4-quinazolinyl]-1H-pyrrole-2,5-dione;
2) 5-(2,4-dihydroxy-5-isopropylphenyl)-N-ethyl-4-(4-(morpholinomethyl)phenyl)isoxazole-3-carboxamide;
3) 2-methyl-2-(4-(3-methyl-2-oxo-8-(quinolin-3-yl)-2,3-dihydro-1H-imidazo[4,5-c]quinolin-1-yl)phenyl)propanenitrile;
4) Compound D;
5) 4-[3,5-bis(2-hydroxyphenyl)-1H-1,2,4-triazol-1-yl]-benzoic acid;
6) 4,4′-(1H-1,2,4-triazol-1-ylmethylene)bis-benzonitrile;
7) (4S,5R)-3-(2′-amino-2-morpholino-4′-(trifluoromethyl)-[4,5′-bipyrimidin]-6-yl)-4-(hydroxymethyl)-5-methyloxazolidin-2-one;
8) (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one;
9) 4-[(4-methyl-1-piperazinyl)methyl]-N-[4-methyl-3-[[4-(3-pyridinyl)-2-pyrimidinyl]amino]phenyl]-methanesulfonate-benzamide;
10) 4-[(R)-6,7-dihydro-5H-pyrrolo[1,2-c]imidazol-5-yl]-3-fluorobenzonitrile;
11) N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate;
12) (R)-2-(5-(4-(6-benzyl-4,5-dimethylpyridazin-3-yl)-2-methylpiperazin-1-yl)pyrazin-2-yl)propan-2-ol;
13) Compound M;
14) 2-(2′,3-dimethyl-[2,4′-bipyridin]-5-yl)-N-(5-(pyrazin-2-yl)pyridin-2-yl)acetamide;
15) 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide;
16) Compound P;
17) Compound Q;
18) N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide;
19) 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine;
20) cyclo((4R)-4-(2-Aminoethylcarbamoyloxy)-L-prolyl-L-phenylglycyl-D-tryptophyl-L-lysyl-4-O-benzyl-L-tyrosyl-L-phenylalanyl-);
21) 1-amino-5-fluoro-3-[6-(4-methyl-1-piperazinyl)-1H-benzimidazol-2-yl]-2(1H)-quinolinone;
22) 8-(6-Methoxy-pyridin-3-yl)-3-methyl-1-(4-piperazin-1-yl-3-trifluoromethyl-phenyl)-1,3-dihydro-imidazo[4,5-c]quinolin-2-one;
23) N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine;
24) 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide;
25) 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine;
26) 5-chloro-N2-(4-(1-ethylpiperidin-4-yl)-2-fluoro-5-methylphenyl)-N⁴-(5-methyl-1H-pyrazol-3-yl)pyrimidine-2,4-diamine;
27) 6-[(2S,4R,6E)-4-Methyl-2-(methylamino)-3-oxo-6-octenoic acid]cyclosporin D, Amdray, PSC833, [3′-Desoxy-3′-oxo-MeBmt]1-[Val]2-cyclosporin;
28) N-(4-Chlorophenyl)-4-(4-pyridinylmethyl)-1-phthalazinamine succinate;
29) Compound CC;
30) (R)—N-(4-(chlorodifluoromethoxy)phenyl)-6-(3-hydroxypyrrolidin-1-yl)-5-(1H-pyrazol-5-yl)nicotinamide;
31) Compound EE;
32) Compound FF; 33) 4-((2-(((1R,2R)-2-hydroxycyclohexyl)amino)benzo[d]thiazol-6-yl)oxy)-N-methylpicolinamide.

### Exemplary Combination Therapies

In one embodiment, the inhibitor of PD-1 is Nivolumab (CAS Registry No: 946414-94-4) disclosed in e.g., U.S. Pat. No. 8,008,449, and having a sequence disclosed herein, e.g., a heavy chain sequence of SEQ ID NO: 2 and a light chain sequence of SEQ ID NO: 3 (or a sequence substantially identical or similar thereto, e.g., a sequence at least 85%, 90%, 95% identical or higher to the sequence specified).

In another embodiment, the inhibitor of PD-1 is Pembrolizumab disclosed in, e.g., U.S. Pat. No. 8,354,509 and International Patent Application Publication No. WO 2009/114335, and having a sequence disclosed herein, e.g., a heavy chain sequence of SEQ ID NO: 4 and a light chain sequence of SEQ ID NO: 5 (or a sequence substantially identical or similar thereto, e.g., a sequence at least 85%, 90%, 95% identical or higher to the sequence specified).

In another embodiment, the inhibitor of PD-L1 is MSB0010718C (also referred to as A09-246-2) disclosed in, e.g., International Patent Application Publication No. WO 2013/0179174, and having a sequence disclosed herein, e.g., a heavy chain sequence of SEQ ID NO: 6 and a light chain sequence of SEQ ID NO: 7 (or a sequence substantially identical or similar thereto, e.g., a sequence at least 85%, 90%, 95% identical or higher to the sequence specified).

In certain embodiments, the PD-1 inhibitor, e.g., the anti-PD-1 antibody (e.g., Nivolumab) is used in a method or composition described herein. For example, the PD-1 inhibitor, e.g., the anti-PD-1 antibody (e.g., Nivolumab or Pembrolizumab); or the PD-L1 inhibitor, e.g., the anti-PD-L1 antibody (e.g., MSB0010718C) (alone or in combination with other immunomodulators) is used in combination with one or more of the agents listed in Table 1, or disclosed in a publication listed in Table 1. In some embodiments, the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor.

In one embodiment, one or more of the aforesaid combinations is used to treat a disorder, e.g., a disorder described herein (e.g., a disorder disclosed in a publication listed in Table 1). Many of the combinations in this section are useful in treating cancer, but other indications are also described. In one embodiment, one or more of the aforesaid combinations is used to treat a cancer, e.g., a cancer described herein (e.g., a cancer disclosed in a publication listed in Table 1). Each of these combinations is discussed in more detail below.

In one embodiment, the inhibitor of the immune checkpoint molecule (alone or in combination with one or more other immunomodulators) is used in combination with a PKC inhibitor to treat a disorder, e.g., a disorder described herein (e.g., a disorder disclosed in a publication listed in Table 1). In one embodiment, the PKC inhibitor is Sotrastaurin (Compound A) as disclosed herein, or in a publication recited in Table 1. In certain embodiments, the PKC inhibitor is disclosed, e.g., in PCT Publication No. WO 2005/039549. In one embodiment, Sotrastaurin (Compound A) has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with Sotrastaurin (Compound A) to treat a disorder described herein, e.g., in a publication recited in Table 1, such as a cancer, a melanoma, a non-Hodgkin lymphoma, an inflammatory bowel disease, transplant rejection, an ophthalmic disorder, or psoriasis.

In certain embodiments, Sotrastaurin (Compound A) is administered at a dose of about 20 to 600 mg, e.g., about 200 to about 600 mg, about 50 mg to about 450 mg, about 100 mg to 400 mg, about 150 mg to 350 mg, or about 200 mg to 300 mg, e.g., about 50 mg, 100 mg, 150 mg, 200 mg, 300 mg, 400 mg, 500 mg, or 600 mg. The dosing schedule can vary from e.g., every other day to daily, twice or three times a day.

In another embodiment, the inhibitor of an immune checkpoint molecule (alone or in combination with one or more other immunomodulators) is used in combination with an HSP90 inhibitor to treat a disorder, e.g., a disorder described herein (e.g., a disorder disclosed in a publication listed in Table 1). In one embodiment, the HSP90 inhibitor is disclosed herein, e.g., in Table 1. In one embodiment, the HSP90 inhibitor is 5-(2,4-dihydroxy-5-isopropylphenyl)-N-ethyl-4-(4-(morpholinomethyl)phenyl)isoxazole-3-carboxamide (Compound B) as disclosed herein, or in a publication recited in Table 1. In certain embodiments, the HSP90 inhibitor is disclosed, e.g., in PCT Publication No. WO 2010/060937 or WO 2004/072051. In one embodiment, Compound B has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with 5-(2,4-dihydroxy-5-isopropylphenyl)-N-ethyl-4-(4-(morpholinomethyl)phenyl)isoxazole-3-carboxamide (Compound B) to treat a disorder described herein, e.g., in a publication recited in Table 1, such as a cancer, a multiple myeloma, a non-small cell lung cancer, a lymphoma, a gastric cancer, a breast cancer, a digestive/gastrointestinal cancer, a pancreatic cancer, a colorectal cancer, a solid tumor, or a hematopoiesis disorder.

In another embodiment, the inhibitor of an immune checkpoint molecule (alone or in combination with one or more other immunomodulators) is used in combination with an inhibitor of PI3K and/or mTOR to treat a disorder, e.g., a disorder described herein (e.g., a disorder disclosed in a publication listed in Table 1). In one embodiment, the inhibitor of PI3K and/or mTOR is disclosed herein, e.g., in Table 1, In one embodiment, the inhibitor of PI3K and/or mTOR is Dactolisib (Compound C) or 8-(6-methoxy-pyridin-3-yl)-3-methyl-1-(4-piperazin-1-yl-3-trifluoromethyl-phenyl)-1,3-dihydro-imidazo[4,5-c]quinolin-2-one (Compound V) as described herein, or in a publication recited in Table 1. In certain embodiments, the inhibitor of PI3K and/or mTOR is disclosed, e.g., in PCT Publication No. WO 2006/122806. In one embodiment, Dactolisib (Compound C) or 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound V) has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with Dactolisib (Compound C) or 8-(6-methoxy-pyridin-3-yl)-3-methyl-1-(4-piperazin-1-yl-3-trifluoromethyl-phenyl)-1,3-dihydro-imidazo[4,5-c]quinolin-2-one (Compound V) to treat a disorder described herein, e.g., in a publication recited in Table 1, such as a cancer, a prostate cancer, a leukemia (e.g., lymphocytic leukemia), a breast cancer, a brain cancer, a bladder cancer, a pancreatic cancer, a renal cancer, a solid tumor, or a liver cancer.

In another embodiment, the inhibitor of an immune checkpoint molecule (alone or in combination with one or more other immunomodulators) is used in combination with an inhibitor of cytochrome P450 (e.g., a CYP17 inhibitor) to treat a disorder, e.g., a disorder described herein (e.g., a disorder disclosed in a publication listed in Table 1). In one embodiment, the cytochrome P450 inhibitor (e.g., the CYP17 inhibitor) is disclosed herein, e.g., in Table 1. In one embodiment, the cytochrome P450 inhibitor (e.g., the CYP17 inhibitor) is Compound D as disclosed herein, e.g., a publication recited in Table 1. In certain embodiments, Compound D is disclosed, e.g., in PCT Publication No. WO 2010/149755. In one embodiment, the inhibitor of immune check point molecule (e.g., one of Nivolumab, Pembrolizumab or MSB0010718C) is used in combination with Compound D to treat a disorder described herein, e.g., in a publication recited in Table 1 to treat a cancer, e.g., a prostate cancer.

In another embodiment, the inhibitor of an immune checkpoint molecule (alone or in combination with one or more other immunomodulators) is used in combination with an iron chelating agent to treat a disorder, e.g., a disorder described herein (e.g., a disorder disclosed in a publication listed in Table 1). In one embodiment, the iron chelating agent is disclosed herein, e.g., in Table 1. In one embodiment, the iron chelating agent is Deferasirox (Compound E) as disclosed herein, or in a publication recited in Table 1. In certain embodiments, the iron chelating agent is disclosed, e.g., in PCT Publication No. WO 1997/049395. In one embodiment, Defeasirox (Compound E) has the structure provided in Table 1, or as disclosed in the publication recited in Table 1). In one embodiment, the inhibitor of immune checkpoint molecule (e.g., one of Nivolumab, Pembrolizumab or MSB0010718C) is used in combination with Deferasirox (Compound E) to treat a disorder described herein, e.g., in a publication recited in Table 1, e.g., iron overload, hemochromatosis, or myelodysplasia.

In another embodiment, the inhibitor of an immune checkpoint molecule (alone or in combination with one or more other immunomodulators) is used in combination with an aromatase inhibitor to treat a disorder, e.g., a disorder described herein (e.g., a disorder disclosed in a publication listed in Table 1). In one embodiment, the aromatase inhibitor is disclosed herein, e.g., in Table 1. In one embodiment, the aromatase inhibitor is Letrozole (Compound F) as disclosed herein, or in a publication recited in Table 1. In certain embodiments, the aromatase inhibitor is disclosed, e.g., in U.S. Pat. No. 4,978,672. In one embodiment, Letrozole (Compound F) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with Letrozole (Compound F) to treat a disorder described herein, e.g., in a publication recited in Table 1, such as a cancer, a leiomyosarcoma, an endometrium cancer, a breast cancer, a female reproductive system cancer, or a hormone deficiency.

In another embodiment, the inhibitor of an immune checkpoint molecule (alone or in combination with one or more other immunomodulators) is used in combination with a PI3K inhibitor, e.g., a pan-PI3K inhibitor, to treat a disorder, e.g., a disorder described herein (e.g., a disorder disclosed in a publication listed in Table 1). In one embodiment, the PI3K inhibitor is disclosed herein, e.g., in Table 1. In one embodiment, the PI3K inhibitor is (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) as disclosed herein, e.g., in a publication recited in Table 1. In certain embodiments, the PI3K inhibitor is disclosed, e.g., in PCT Publication No. WO2013/124826. In one embodiment, (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) has the structure provided in Table 1, or as disclosed in a publication recited in Table 1. In one embodiment, the immune checkpoint molecule (e.g., one of Nivolumab, Pembrolizumab or MSB0010718C) is used in combination with (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) to treat a disorder described herein, e.g., in a publication recited in Table 1, such as a cancer or an advanced solid tumor.

In another embodiment, the inhibitor of an immune checkpoint molecule (alone or in combination with one or more other immunomodulators) is used in combination with an inhibitor of p53 and/or a p53/Mdm2 interaction to treat a disorder, e.g., a disorder described herein (e.g., a disorder disclosed in a publication listed in Table 1). In one embodiment, the p53 and/or a p53/Mdm2 interaction inhibitor is disclosed herein, e.g., in Table 1. In one embodiment, the p53 and/or a p53/Mdm2 interaction inhibitor is (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) as disclosed herein, or in a publication recited in Table 1. In certain embodiments, the p53 and/or a p53/Mdm2 interaction inhibitor is disclosed, e.g., in PCT Publication No. WO2013/111105. In one embodiment, (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) to treat a disorder described herein, e.g., in publication recited in Table 1, such as a cancer or a soft tissue sarcoma.

In another embodiment, the inhibitor of an immune checkpoint molecule (alone or in combination with one or more other immunomodulators) is used in combination with an apoptosis inducer and/or an angiogenesis inhibitor to treat a disorder, e.g., a disorder described (e.g., a disorder disclosed in a publication listed in Table 1). In one embodiment, the apoptosis inducer and/or an angiogenesis inhibitor is disclosed herein, e.g., in Table 1. In one embodiment, the apoptosis inducer and/or an angiogenesis inhibitor is Imatinib mesylate (Compound I) as disclosed herein, or in a publication recited in Table 1. In certain embodiments, the apoptosis inducer and/or an angiogeneisis inhibitor is disclosed, e.g., in PCT Publication No. WO1999/003854. In one embodiment, the apoptosis inducer and/or an angiogenesis inhibitor has the structure provided in Table 1, or as disclosed in a publication disclosed in Table 1. In one embodiment, the inhibitor of immune checkpoint molecule (e.g., one of Nivolumab, Pembrolizumab or MSB0010718C) is used in combination with Imatinib mesylate (Compound I) to treat a disorder described herein, e.g., in a publication recited in Table 1, such as a cancer, a multiple myeloma, a prostate cancer, a non-small cell lung cancer, a lymphoma, a gastric cancer, a melanoma, a breast cancer, a pancreatic cancer, a digestive/gastrointestinal cancer, a colorectal cancer, a glioblastoma multiforme, a liver cancer, a head and neck cancer, asthma, multiple sclerosis, allergy, Alzheimer's dementia, amyotrophic lateral sclerosis, or rheumatoid arthritis.

In certain embodiments, Imatinib mesylate (Compound I) is administered at a dose of about 100 to 1000 mg, e.g., about 200 mg to 800 mg, about 300 mg to 700 mg, or about 400 mg to 600 mg, e.g., about 200 mg, 300 mg, 400 mg, 500 mg, 600 mg, or 700 mg. The dosing schedule can vary from e.g., every other day to daily, twice or three times a day. In one embodiment, Imatinib mesylate is administered at an oral dose from about 100 mg to 600 mg daily, e.g., about 100 mg, 200 mg, 260 mg, 300 mg, 400 mg, or 600 mg daily.

In another embodiment, the inhibitor of an immune checkpoint molecule (alone or in combination with one or more other immunomodulators) is used in combination with an inhibitor of one or more of cytochrome P450 (e.g., 11B2), aldosterone or angiogenesis to treat a disorder, e.g., a disorder described herein (e.g., in a disorder disclosed in a publication listed in Table 1). In one embodiment, the inhibitor of one or more of cytochrome P450 (e.g., 11B2), aldosterone or angiogenesis is disclosed herein, e.g., in Table 1. In one embodiment, the inhibitor of one or more of cytochrome P450 (e.g., 11B2), aldosterone or angiogenesis is Osilodrostat (Compound J) as disclosed herein, or in a publication recited in Table 1. In certain embodiments, the inhibitor of one or more of cytochrome P450 (e.g., 11B2), aldosterone or angiogenesis is disclosed, e.g., in PCT Publication No. WO2007/024945. In one embodiment, Osilodrostat (Compound J) has the structure provided in Table 1, or as disclosed in a publication recited in Table 1. In one embodiment, the immune checkpoint molecule (e.g., one of Nivolumab, Pembrolizumab or MSB0010718C) is used in combination with Osilodrostat (Compound J) to treat a disorder described herein, e.g., in a publication recited in Table 1, such as Cushing's syndrome, hypertension, or heart failure therapy.

In another embodiment, the inhibitor of an immune checkpoint molecule (alone or in combination with one or more other immunomodulators) is used in combination a Smoothened (SMO) inhibitor to treat a disorder, e.g., a disorder described herein (e.g., a disorder disclosed in a publication listed in Table 1 In one embodiment, the SMO inhibitor is disclosed herein, e.g., in Table 1. In one embodiment, the SMO inhibitor is Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) as disclosed herein, or in a publication recited in Table 1. In certain embodiments, the SMO inhibitor is disclosed, e.g., in PCT Publication No. WO 2007/131201 or WO 2010/007120. In certain embodiments, Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) to treat a disorder described herein, in a publication recited in Table 1, such as a cancer, a medulloblastoma, a small cell lung cancer, a prostate cancer, a basal cell carcinoma, a pancreatic cancer, or an inflammation.

In certain embodiments, Sonidegib phosphate (Compound K) is administered at a dose of about 20 to 500 mg, e.g., about 40 mg to 400 mg, about 50 mg to 300 mg, or about 100 mg to 200 mg, e.g., about 50 mg, 100 mg, 150 mg, 200 mg, 250 mg, or 300 mg. The dosing schedule can vary from e.g., every other day to daily, twice or three times a day.

In another embodiment, the inhibitor of an immune checkpoint molecule (alone or in combination with one or more other immunomodulators) is used in combination a prolactin receptor (PRLR) inhibitor to treat a disorder, e.g., a disorder described herein (e.g., a disorder disclosed in a publication listed in Table 1). In one embodiment, the PRLR inhibitor is disclosed herein, e.g., in Table 1. In one embodiment, the PRLR inhibitor is a human monoclonal antibody (Compound M) disclosed herein, e.g., or in a publication recited in Table 1. In certain embodiments, the human monoclonal antibody (Compound M) is disclosed, e.g., in U.S. Pat. No. 7,867,493. In one embodiment, the inhibitor of the immune checkpoint molecule (e.g., one of Nivolumab, Pembrolizumab or MSB0010718C) is used in combination with human monoclonal antibody molecule (Compound M) described in U.S. Pat. No. 7,867,493 to treat a disorder such as, a cancer, a prostate cancer, or a breast cancer.

In another embodiment, the inhibitor of an immune checkpoint molecule (alone or in combination with one or more other immunomodulators) is used in combination a Wnt signaling inhibitor to treat a disorder, e.g., a disorder described herein (e.g., a disorder disclosed in a publication listed in Table 1). In one embodiment, the Wnt signaling inhibitor is disclosed herein, e.g., in Table 1. In one embodiment, the Wnt signaling inhibitor is 2-(2′,3-dimethyl-[2,4′-bipyridin]-5-yl)-N-(5-(pyrazin-2-yl)pyridin-2-yl)acetamide (Compound N) as disclosed herein, or in a publication recited in Table 1. In certain embodiments, 2-(2′,3-dimethyl-[2,4′-bipyridin]-5-yl)-N-(5-(pyrazin-2-yl)pyridin-2-yl)acetamide (Compound N) is disclosed, e.g., in PCT publication No. WO 2010/101849. In one embodiment, the inhibitor of the immune checkpoint molecule (e.g., one of Nivolumab, Pembrolizumab or MSB0010718C) is used in combination with 2-(2′,3-dimethyl-[2,4′-bipyridin]-5-yl)-N-(5-(pyrazin-2-yl)pyridin-2-yl)acetamide (Compound N) to treat a disorder described herein, in a publication disclosed in Table 1, such as a cancer or a solid tumor (e.g., a head and neck cancer, a squamous cell carcinoma, a breast cancer, a pancreatic cancer, or a colon cancer).

In certain embodiments, 2-(2′,3-dimethyl-[2,4′-bipyridin]-5-yl)-N-(5-(pyrazin-2-yl)pyridin-2-yl)acetamide (Compound N) is administered at a dose of about 1 to 50 mg, e.g., about 2 mg to 45 mg, about 3 mg to 40 mg, about 5 mg to 35 mg, 5 mg to 10 mg, or about 10 mg to 30 mg, e.g., about 2 mg, 5 mg, 10 mg, 20 mg, 30 mg, or 40 mg. The dosing schedule can vary from e.g., every other day to daily, twice or three times a day.

In another embodiment, the inhibitor of an immune checkpoint molecule (alone or in combination with one or more other immunomodulators) is used in combination a CDK4/6 inhibitor to treat a disorder, e.g., a disorder described herein (e.g., a disorder disclosed in a publication listed in Table 1). In one embodiment, the CDK4/6 inhibitor is disclosed herein, e.g., in Table 1. In one embodiment, the CDK4/6 inhibitor is 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) as disclosed herein in a publication recited in Table 1. In certain embodiments, the CDK4/6 inhibitor is disclosed in PCT publication No. WO 2011/101409. In certain embodiments, 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) has the structure provided in Table 1, or in a publication recited in Table 1. In one embodiment, the inhibitor of the immune checkpoint molecule (e.g., one of Nivolumab, Pembrolizumab or MSB0010718C) is used in combination with 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) to treat a disorder described herein, e.g., in a publication recited in Table 1, such as a cancer, a mantle cell lymphoma, a liposarcoma, a non-small cell lung cancer, a melanoma, a squamous cell esophageal cancer, or a breast cancer.

In another embodiment, the inhibitor of an immune checkpoint molecule (alone or in combination with one or more other immunomodulators) is used in combination an FGFR2 and/or FGFR4 inhibitor to treat a disorder, e.g., a disorder described herein (e.g., a disorder disclosed in a publication recited in Table 1). In one embodiment, the FGFR2 and/or FGFR4 inhibitor is disclosed herein, e.g., in Table 1. In one embodiment, the FGFR2 and/or FGFR4 inhibitor is an antibody molecule drug conjugate against an FGFR2 and/or FGFR4 (e.g., mAb 12425 or Compound P) disclosed herein, or in a publication disclosed in Table 1. In certain embodiments, the FGFR2 and/or FGFR4 inhibitor is disclosed, e.g., in PCT Publication No. WO 2014/160160. In one embodiment, the FGFR2 and/or FGFR4 inhibitor (e.g., one of Nivolumab, Pembrolizumab or MSB0010718C) is used in combination with an antibody molecule drug conjugate against an FGFR2 and/or FGFR4 (e.g., mAb 12425 or Compound P) to treat a disorder described herein, e.g., in a publication recited in Table 1, such as a cancer, a gastric cancer, a breast cancer, a rhabdomyosarcoma, a liver cancer, an adrenal cancer, a lung cancer, an esophageal cancer, a colon cancer, or an endometrial cancer.

In some embodiments, Compound P is an antibody molecule drug conjugate against an FGFR2 and/or FGFR4, e.g., mAb 12425.

In another embodiment, the inhibitor of an immune checkpoint molecule (alone or in combination with one or more other immunomodulators) is used in combination an M-CSF inhibitor to treat a disorder, e.g., a disorder described herein (e.g., a disorder disclosed in a publication listed in Table 1). In one embodiment, the M-CSF inhibitor is disclosed herein, e.g., in Table 1. In one embodiment, the M-CSF inhibitor is an antibody molecule or Fab fragment against M-CSF (e.g., Compound Q) disclosed herein, or in a publication recited in Table 1. In certain embodiments, the antibody molecule or Fab fragment against M-CSF (e.g., Compound Q) is disclosed in PCT Publication No. WO 2004/045532. In one embodiment, the inhibitor of the immune checkpoint molecule (e.g., one of Nivolumab, Pembrolizumab or MSB0010718C) is used in combination with the antibody molecule or Fab fragment against M-CSF (e.g., Compound Q) to treat a disorder described herein, e.g., in a publication recited in Table 1, such as a cancer, a prostate cancer, a breast cancer, or pigmented villonodular synovitis (PVNS).

In embodiments, Compound Q is a monoclonal antibody molecule against M-CSF or a fragment (e.g., Fab fragment) thereof. In embodiments, the M-CSF inhibitor or Compound Q is administered at an average dose of about 10 mg/kg.

In another embodiment, the inhibitor of an immune checkpoint molecule (alone or in combination with one or more other immunomodulators) is used in combination an inhibitor of one or more of c-KIT, histamine release, Flt3 (e.g., FLK2/STK1) or PKC to treat a disorder, e.g., a disorder described herein (e.g., a disorder disclosed in a publication listed in Table 1). In one embodiment, the inhibitor of one or more of c-KIT, histamine release, Flt3 (e.g., FLK2/STK1) or PKC is disclosed herein, e.g., in Table 1. In one embodiment, the inhibitor of one or more of c-KIT, histamine release, Flt3 (e.g., FLK2/STK1) or PKC is Midostaurin (Compound R) disclosed herein, e.g., in a publication recited in Table 1. In certain embodiments, the inhibitor of one or more of c-KIT, histamine release, Flt3 (e.g., FLK2/STK1) or PKC is disclosed in PCT Publication No. WO 2003/037347. In one embodiment, Midostaurin (Compound R) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Midostaurin (Compound R) to treat a disorder described herein, e.g., in a publication recited in Table 1, such as a cancer, a colorectal cancer, a myeloid leukemia, myelodysplastic syndrome, an age-related mascular degeration, a diabetic complication, or a dermatologic disorder.

In another embodiment, the inhibitor of an immune checkpoint molecule (alone or in combination with one or more other immunomodulators) is used in combination an inhibitor of one or more of VEGFR-2, PDGFRbeta, KIT or Raf kinase C to treat a disorder, e.g., a disorder described herein (e.g., a disorder in a publication listed in Table 1). In one embodiment, the inhibitor of one or more of VEGFR-2, PDGFRbeta, KIT or Raf kinase C is disclosed herein, e.g., in Table 1. In one embodiment, the inhibitor of one or more of VEGFR-2, PDGFRbeta, KIT or Raf kinase C is 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) as disclosed herein, e.g., in a publication recited in Table 1. In certain embodiments, the inhibitor of one or more of VEGFR-2, PDGFRbeta, KIT or Raf kinase C is disclosed, e.g., in PCT Publication No. WO 2007/030377. In one embodiment, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) to treat a disorder described herein, e.g., in a publication recited in Table 1, such as a cancer, a melanoma, or a solid tumor.

In another embodiment, the inhibitor of an immune checkpoint molecule (alone or in combination with one or more other immunomodulators) is used in combination a somatostatin agonist and/or growth hormone release inhibitor to treat a disorder, e.g., a disorder described herein (e.g., a disorder disclosed in a publication listed in Table 1). In one embodiment, the somatostatin agonist and/or growth hormone release inhibitor is disclosed herein, e.g., in Table 1. In one embodiment, the somatostatin agonist and/or growth hormone release inhibitor is Pasireotide diaspartate (Compound T) disclosed herein, e.g., in a publication recited in Table 1. In certain embodiments, the somatostatin agonist and/or growth hormone release inhibitor is disclosed, e.g., in PCT Publication No. WO2002/010192 or U.S. Pat. No. 7,473,761. In one embodiment, Pasireotide diaspartate (Compound T) has the structure provided in Table 1, or in a publication recited in Table 1. In one embodiment, the inhibitor of the immune checkpoint molecule (e.g., one of Nivolumab, Pembrolizumab or MSB0010718C) is used in combination with Pasireotide diaspartate (Compound T) to treat a disorder described herein, e.g., in a publication recited in Table 1, such as a cancer, a prostate cancer, an endocrine cancer, a nurologic cancer, a skin cancer (e.g., a melanoma), a pancreatic cancer, a liver cancer, Cushing's syndrome, a gastrointestinal disorder, acromegaly, a liver and biliary tract disorder, or liver cirrhosis.

In another embodiment, the inhibitor of an immune checkpoint molecule (alone or in combination with one or more other immunomodulators) is used in combination a signal transduction modulator and/or angiogenesis inhibitor to treat a disorder, e.g., a disorder described herein (e.g., a disorder disclosed in a publication listed in Table 1). In one embodiment, the signal transduction modulator and/or angiogenesis inhibitor is disclosed herein, e.g., in Table 1. In one embodiment, the signal transduction modulator and/or angiogenesis inhibitor is Dovitinib (Compound U) as disclosed herein, or in a publication recited in Table 1. In certain embodiments, the signal transduction modulator and/or angiogenesis inhibitor is disclosed, e.g., in PCT Publication No. WO 2009/115562. In one embodiment, Dovitinib (Compound U) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Dovitinib (Compound U) to treat a disorder described herein, e.g., in a publication recited in Table 1, such as a cancer, a respiratory/thoracic cancer, a multiple myeloma, a prostate cancer, a non-small cell lung cancer, an endocrine cancer, or a neurological genetic disorder.

In another embodiment, the inhibitor of an immune checkpoint molecule (alone or in combination with one or more other immunomodulators) is used in combination an ALK inhibitor to treat a disorder, e.g., a disorder described herein (e.g., a disorder disclosed in a publication listed in Table 1). In one embodiment, the ALK inhibitor is disclosed herein, e.g., in Table 1. In one embodiment, the ALK inhibitor is N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) as disclosed herein, or in a publication recited in Table 1. In certain embodiments, the ALK inhibitor is disclosed in PCT Publication No. WO 2008/073687. In one embodiment, N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) has the structure provided in Table 1, or as disclosed in a publication recited in Table 1. In one embodiment, the inhibitior of the immune checkpoint molecule (e.g., one of Nivolumab, Pembrolizumab or MSB0010718C) is used in combination with N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) to treat a disorder described herein, e.g., in a publication recited in Table 1, such as a cancer, an anaplastic large-cell lymphoma (ALCL), a non-small cell lung carcinoma (NSCLC), or a neuroblastoma.

In another embodiment, the inhibitor of an immune checkpoint molecule (alone or in combination with one or more other immunomodulators) is used in combination an IGF-1R inhibitor to treat a disorder, e.g., a disorder described (e.g., a disorder disclosed in a publication listed in Table 1). In one embodiment, the IGF-1R inhibitor is disclosed herein, e.g., in a publication recited in Table 1. In one embodiment, the IGF-1R inhibitor is 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound Z), as disclosed herein, or in a publication recited in Table 1. In certain embodiments, the IGF-1R inhibitor is disclosed, e.g., in PCT Publication No. WO 2010/002655. In one embodiment, 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound Z) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N2-(4-(1-ethylpiperidin-4-yl)-2-fluoro-5-methylphenyl)-N⁴-(5-methyl-1H-pyrazol-3-yl)pyrimidine-2,4-diamine (Compound Z) to treat a disorder described herein, e.g., in a publication recited in Table 1, such as a cancer or a sarcoma.

In another embodiment, the inhibitor of an immune checkpoint molecule (alone or in combination with one or more other immunomodulators) is used in combination a P-Glycoprotein 1 inhibitor to treat a disorder, e.g., a disorder described herein (e.g., a disorder disclosed in a publication listed in Table 1). In one embodiment, the P-Glycoprotein 1 inhibitor is disclosed herein, e.g., in Table 1. In one embodiment, the P-Glycoprotein 1 inhibitor is Valspodar (Compound AA) as disclosed herein, or in a publication recited in Table 1. In certain embodiments, the P-Glycoprotein 1 inhibitor is disclosed, e.g., in EP 296122. In one embodiment, Valspodar (Compound AA) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Valspodar (Compound AA) to treat a disorder described herein, e.g., in a publication recited in Table 1, such as a cancer or a drug-resistant tumor.

In another embodiment, the inhibitor of an immune checkpoint molecule (alone or in combination with one or more other immunomodulators) is used in combination with a VEGFR inhibitor to treat a disorder, e.g., a disorder described herein (e.g., a disorder disclosed in a publication listed in Table 1). In one embodiment, the VEGFR inhibitor is disclosed herein, e.g., in Table 1. In one embodiment, the VEGFR inhibitor is Vatalanib succinate (Compound BB) as disclosed herein, or in a publication recited in Table 1. In certain embodiments, the VEGFR inhibitor is disclosed, e.g., in WO 98/35958. In one embodiment, Vatalanib succinate (Compound BB) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Vatalanib succinate (Compound BB) to treat a disorder described herein, e.g., in a publication recited in Table 1, such as a cancer.

In another embodiment, the inhibitor of an immune checkpoint molecule (alone or in combination with one or more other immunomodulators0 is used in combination with an IDH inhibitor to treat a disorder, e.g., a disorder described herein (e.g., a disorder disclosed in a publication listed in Table 1). In one embodiment, the IDH inhibitor is disclosed herein, e.g., in Table 1. In one embodiment, the IDH inhibitor is Compound CC as disclosed in Table 1, or in a publication recited in Table 1. In one embodiment, the IDH inhibitor is disclosed, e.g., in PCT Publication No. WO2014/141104. In one embodiment, the inhibitor of the immune checkpoint molecule (e.g., one of Nivolumab, Pembrolizumab or MSB0010718C) is used in combination with Compound CC to treat a disorder described herein, e.g., in a publication recited in Table 1, such as a cancer.

In another embodiment, the inhibitor of an immune checkpoint molecule (alone or in combination with one or more other immunomodulators) is used in combination with a BCL-ABL inhibitor to treat a disorder, e.g., a disorder described herein (e.g., a disorder disclosed in a publication listed in Table 1). In one embodiment, the BCL-ABL inhibitor is disclosed herein, e.g., in Table 1. In one embodiment, the BCL-ABL inhibitor is (R)—N-(4-(chlorodifluoromethoxy)phenyl)-6-(3-hydroxypyrrolidin-1-yl)-5-(1H-pyrazol-5-yl)nicotinamide (Compound DD) as disclosed in Table 1, or in a publication recited in Table 1. In certain embodiments, (R)—N-(4-(chlorodifluoromethoxy)phenyl)-6-(3-hydroxypyrrolidin-1-yl)-5-(1H-pyrazol-5-yl)nicotinamide (Compound DD) is disclosed, e.g., in PCT Publication No. WO2013/171639, WO2013/171640, WO2013/171641, or WO2013/171642. In one embodiment, Compound DD has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Compound DD to treat a disorder described herein, e.g., in a publication recited in Table 1, such as a cancer.

In another embodiment, the inhibitor of an immune checkpoint molecule (alone or in combination with one or more other immunomodulators) is used in combination with a c-RAF inhibitor to treat a disorder, e.g., a disorder described herein (e.g., a disorder disclosed in a publication listed in Table 1). In one embodiment, the c-RAF inhibitor is disclosed herein, e.g., in Table 1. In one embodiment, the c-RAF inhibitor is Compound EE as disclosed herein, or in a publication recited in Table 1. In certain embodiments, Compound EE is disclosed in PCT Publication No. WO2014/151616. In one embodiment, the inhibitor of the immune checkpoint molecule (e.g., one of Nivolumab, Pembrolizumab or MSB0010718C) is used in combination with Compound EE to treat a disorder described herein, e.g., in a publication recited in Table 1, such as a cancer.

In another embodiment, the inhibitor of an immune checkpoint molecule (alone or in combination with one or more other immunomodulators) is used in combination with an ERK1/2 ATP competitive inhibitor to treat a disorder, e.g., a disorder described herein (e.g., a disorder disclosed in a publication listed in Table 1). In one embodiment, the ERK1/2 ATP competitive inhibitor is disclosed herein, e.g., in Table 1. In one embodiment, the ERK1/2 ATP competitive inhibitor is Compound FF as disclosed herein, or in a publication recited in Table 1. In certain embodiments, Compound FF is disclosed in International Patent Application No. PCT/US2014/062913. In one embodiment, Compound FF has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Compound FF to treat a disorder described herein, e.g., in a publication recited in Table 1, such as a cancer.

In another embodiment, the inhibitor of an immune checkpoint molecule (alone or in combination with one or more other immunomodulators) is used in combination a CSF-1R tyrosine kinase inhibitor to treat a disorder, e.g., a disorder described herein (e.g., a disorder disclosed in a publication listed in Table 1). In one embodiment, the CSF-1R tyrosine kinase inhibitor is disclosed herein, e.g., in Table 1. In one embodiment, the CSF-1R tyrosine kinase inhibitor is 4-((2-(((1R,2R)-2-hydroxycyclohexyl)amino)benzo[d]thiazol-6-yl)oxy)-N-methylpicolinamide (Compound GG) as disclosed herein, or in a publication recited in Table 1. In certain embodiments, the CSF-1R tyrosine kinase inhibitor is disclosed, e.g., in PCT Publication No. WO2005/073224. In one embodiment, 4-((2-(((1R,2R)-2-hydroxycyclohexyl)amino)benzo[d]thiazol-6-yl)oxy)-N-methylpicolinamide (Compound GG) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 4-((2-(((1R,2R)-2-hydroxycyclohexyl)amino)benzo[d]thiazol-6-yl)oxy)-N-methylpicolinamide (Compound GG) to treat a disorder described herein, e.g., in a publication recited in Table 1, such as a cancer.

In some embodiments, the immunomodulator, e.g., the inhibitor of an immune checkpoint molecule as described herein, is administered in combination with Compound Q.

In some embodiments, the immunomodulator, e.g., the inhibitor of an immune checkpoint molecule as described herein, is administered in combination with an anti-cancer agent having a known activity in an immune cell assay, e.g., in one or more of a huMLR assay, a T cell proliferation assay, and a B-cell proliferation assay. Exemplary assays are described below. Based on the assay, an IC50 for can be calculated for each test agent. In some embodiments, the anti-cancer agent has an IC50 of, e.g., 0-1 μM, 1-4 μM, or greater than 4 μM, e.g., 4-10 μM or 4-20 μM. In embodiments, the second therapeutic agent is chosen from one or more of: Compound D, Compound I, Compound K, Compound L, Compound N, Compound CC and Compound DD.

In some embodiments, the Compound N (or a compound related to Compound N) is administered at a dose of approximately 5-10 or 10-30 mg. In some embodiments, the Compound K (or compound related to Compound K) is administered at a dose of about 200 mg. In some embodiments, the Compound I (or compound related to Compound I) is administered at a dose of approximately 400-600 mg PO qDay. In some embodiments, the Compound A (or compound related to Compound A) is administered at a dose of approximately 200-300 or 200-600 mg. In embodiments, the BCR-ABL inhibitor is administered at a dose of approximately 20 mg bid-80 mg bid.

### Cancers and Subjects

In certain embodiments of the compositions and methods described herein, the hyperproliferative disorder or condition, e.g., the cancer, includes but is not limited to, a solid tumor, a soft tissue tumor (e.g., a hematological cancer, leukemia, lymphoma, or myeloma), and a metastatic lesion of any of the aforesaid cancers. In one embodiment, the cancer is a solid tumor. Examples of solid tumors include malignancies, e.g., sarcomas, adenocarcinomas, and carcinomas, of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

In one embodiment, the cancer is chosen from a cancer disclosed in a publication listed in Table 1. For example, the cancer can be chosen from a solid tumor, e.g., a lung cancer (e.g., a non-small cell lung cancer (NSCLC) (e.g., a NSCLC with squamous and/or non-squamous histology, or a NSCLC adenocarcinoma), a small cell lung cancer), a colorectal cancer, a melanoma (e.g., an advanced melanoma), a brain cancer (e.g., glioblastoma multiforme), a head and neck cancer (e.g., head and neck squamous cell carcinoma (HNSCC), a digestive/gastrointestinal cancer, a gastric cancer, a neurologic cancer, a glioblastoma (e.g., glioblastoma multiforme), an ovarian cancer, a renal cancer, a liver cancer, a pancreatic cancer, an esophageal cancer, an endocrine cancer, a respiratory/thoracic cancer, a prostate cancer, a liver cancer; a breast cancer, an anal cancer, a gastro-esophageal cancer, a thyroid cancer, a cervical cancer, an endometrial cancer; or a hematological cancer (e.g., a multiple myoloma, a lymphoa or a leukemia chosen from a Hogdkin's lymphoma, a non-Hodgkin's lymphoma, a lymphocytic leukemia, or a myeloid leukemia).

In one embodiment, the cancer is a non-small cell lung cancer (NSCLC), e.g., an ALK+ NSCLC. As used herein, the term “ALK+ non-small cell lung cancer” or “ALK+ NSCLC” refers to an NSCLC that has an activated (e.g., constitutively activated) anaplastic lymphoma kinase activity or has a rearrangement or translocation of an Anaplastic Lymphoma Kinase (ALK) gene. Typically, compared with the general NSCLC population, patients with ALK+ NSCLC are generally younger, have light (e.g., <10 pack years) or no smoking history, present with lower Eastern Cooperative Oncology Group performance status, or may have more aggressive disease and, therefore, experience earlier disease progression (Shaw et al. J Clin Oncol. 2009; 27(26):4247-4253; Sasaki et al. Eur J Cancer. 2010; 46(10):1773-1780; Shaw et al. N Engl J Med. 2013; 368(25):2385-2394; Socinski et al. J Clin Oncol. 2012; 30(17):2055-2062; Yang et al. J Thorac Oncol. 2012; 7(1):90-97).

In one embodiment, the cancer, e.g., an NSCLC, has a rearrangement or translocation of an ALK gene. In one embodiment, the rearrangement or translocation of the ALK gene leads to a fusion (e.g., fusion upstream of the ALK promoter region). In certain embodiments, the fusion results in constitutive activation of the kinase activity.

In one embodiment, the fusion is an EML4-ALK fusion. Exemplary EML4-ALK fusion proteins include, but are not limited to, E13;A20 (V1), E20;A20 (V2), E6a/b;A20 (V3a/b), E14;A20 (V4), E2a/b;A20 (V5a/b), E13b;A20 (V6), E14;A20(V7), E15;A20(“V4”), or E18;A20 (V5) (Choi et al. Cancer Res. 2008; 68(13):4971-6; Horn et al. J Clin Oncol. 2009; 27(26):4232-5; Koivunen et al. Clin Cancer Res. 2008; 14(13):4275-83; Soda et al. Nature. 2007; 448(7153):561-6; Takeuchi et al. Clin Cancer Res. 2008; 14(20):6618-24; Takeuchi et al. Clin Cancer Res. 2009; 15(9):3143-9; Wong et al. Cancer. 2009 Apr. 15; 115(8):1723-33).

In certain embodiments, the ALK gene is fused to a non-EML4 partner. In one embodiment, the fusion is a KIFSB-ALK fusion. In another embodiment, the fusion is a TFG-ALK fusion. Exemplary KIFSB-ALK and TFG-ALK fusions are described, e.g., in Takeuchi et al. Clin Cancer Res. 2009; 15(9):3143-9, Rikova et al. Cell. 2007; 131(6):1190-203. ALK gene rearrangements or translocations, or cancer cells that has an ALK gene rearrangement or translocation, can be detected, e.g., using fluorescence in situ hybridization (FISH), e.g., with an ALK break apart probe.

Methods and compositions disclosed herein are useful for treating metastatic lesions associated with the aforementioned cancers.

In other embodiments, the subject is a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In one embodiment, the subject (e.g., a subject having a lung cancer (e.g., a non-small cell lung cancer), a lymphoma (e.g., an anaplastic large-cell lymphoma or non-Hodgkin lymphoma), an inflammatory myofibroblastic tumor, or a neuroblastoma) is being treated, or has been treated, with another ALK inhibitor and/or a ROS1 inhibitor, e.g., crizotinib. For example, crizotinib can be administered at a daily oral dose of 750 mg or lower, e.g., 600 mg or lower, e.g., 450 mg or lower.

In another embodiment, the subject or cancer (e.g., a lung cancer (e.g., a non-small cell lung cancer), a lymphoma (e.g., an anaplastic large-cell lymphoma or non-Hodgkin lymphoma), an inflammatory myofibroblastic tumor, or a neuroblastoma) has progressed on, or is resistant or tolerant to, another ALK inhibitor and/or a ROS1 inhibitor, e.g., crizotinib.

In yet another embodiment, the subject or cancer (e.g., a lung cancer (e.g., a non-small cell lung cancer), a lymphoma (e.g., an anaplastic large-cell lymphoma or non-Hodgkin lymphoma), an inflammatory myofibroblastic tumor, or a neuroblastoma) is at risk of progression on, or developing resistance or tolerance to, another ALK inhibitor and/or a ROS1 inhibitor, e.g., crizotinib.

In other embodiments, the subject or cancer is resistant or tolerant, or is at risk of developing resistance or tolerance, to a tyrosine kinase inhibitor (TKI), e.g., an EGFR tyrosine kinase inhibitor. In some embodiments, the subject or cancer has no detectable EGFR mutation, KRAS mutation, or both.

In some embodiments, the subject has previously been treated with a PD1 and/or PD-L1 inhibitor.

In one embodiment, the cancer microenvironment has an elevated level of PD-L1 expression. Alternatively, or in combination, the cancer microenvironment can have increased IFNγ and/or CD8 expression.

In some embodiments, the subject has, or is identified as having, a tumor that has one or more of high PD-L1 level or expression, or as being Tumor Infiltrating Lymphocyte (TIL)+(e.g., as having an increased number of TILs), or both. In certain embodiments, the subject has, or is identified as having, a tumor that has high PD-L1 level or expression and that is TIL+. In some embodiments, the methods described herein further include identifying a subject based on having a tumor that has one or more of high PD-L1 level or expression, or as being TIL+, or both. In certain embodiments, the methods described herein further include identifying a subject based on having a tumor that has high PD-L1 level or expression and as being TIL+. In some embodiments, tumors that are TIL+ are positive for CD8 and IFNγ. In some embodiments, the subject has, or is identified as having, a high percentage of cells that are positive for one, two or more of PD-L1, CD8, and/or IFNγ. In certain embodiments, the subject has or is identified as having a high percentage of cells that are positive for all of PD-L1, CD8, and IFNγ.

In some embodiments, the methods described herein further include identifying a subject based on having a high percentage of cells that are positive for one, two or more of PD-L1, CD8, and/or IFNγ. In certain embodiments, the methods described herein further include identifying a subject based on having a high percentage of cells that are positive for all of PD-L1, CD8, and IFNγ. In some embodiments, the subject has, or is identified as having, one, two or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; an esophageal cancer; a thyroid cancer; a melanoma, and/or a nasopharyngeal cancer (NPC). In certain embodiments, the methods described herein further describe identifying a subject based on having one, two or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; a thyroid cancer; a melanoma, and or a nasopharyngeal cancer.

Methods and compositions disclosed herein are useful for treating metastatic lesions associated with the aforementioned cancers. Treatment of metastatic cancers, e.g., metastatic cancers that express PD-L1 (Iwai et al. (2005) Int. Immunol. 17:133-144) can be effected using the antibody molecules described herein. In one embodiment, the cancer expresses an elevated level of PD-L1, IFNγ and/or CD8.

While not wishing to be bound by theory, in some embodiments, a patient is more likely to respond to treatment with an immunomodulator (optionally in combination with one or more agents as described herein) if the patient has a cancer that highly expresses PD-L1, and/or the cancer is infiltrated by anti-tumor immune cells, e.g., TILs. The anti-tumor immunce cells may be positive for CD8, PD-L1, and/or IFN-γ; thus levels of CD8, PD-L1, and/or IFN-γ can serve as a readout for levels of TILs in the microenvironment. In certain embodiments, the cancer microenvironment is referred to as triple-positive for PD-L1/CD8/IFN-γ.

Accordingly, in certain aspects, this application provides methods of determining whether a tumor sample is positive for one or more of PD-L1, CD8, and IFN-γ, and if the tumor sample is positive for one or more, e.g., two, or all three, of the markers, then administering to the patient a therapeutically effective amount of an anti-PD-1 antibody molecule or an anti-PD-L1 antibody molecule, e.g., an anti-PD-1 antibody molecule or an anti-PD-L1 antibody molecule described herein, optionally in combination with one or more other immunnomodulators or anti-cancer agents.

In the following indications, a large fraction of patients are triple-positive for PD-L1/CD8/IFN-γ: Lung cancer (squamous); lung cancer (adenocarcinoma); head and neck cancer; stomach cancer; NSCLC; HNSCC; gastric cancers (e.g., MSIhi and/or EBV+); CRC (e.g., MSIhi); nasopharyngeal cancer (NPC); cervical cancer (e.g., squamous); thyroid cancer e.g., papillary thyroid; melanoma; TN breast cancer; and DLBCL (Diffuse Large B-Cell Lymphoma). In breast cancer generally and in colon cancer generally, a moderate fraction of patients is triple-positive for PD-L1/CD8/IFN-γ. In the following indications, a small fraction of patients are triple-positive for PD-L1/CD8/IFN-γ: ER+ breast cancer, and pancreatic cancer. These findings are discussed further in Example 1. Regardless of whether a large or small fraction of patients is triple-positive for these markers, screening the patients for these markers allows one to identify a fraction of patients that has an especially high likelihood of responding favorably to therapy with a PD-1 or PD-L1 antibody (e.g., a blocking PD-1 or PD-L1 antibody, e.g., a PD-1 or PD-L1 antibody described herein), optionally in combination with one or more other immunomodulators (e.g., an anti-TIM-3 antibody molecule, an anti-LAG-3 antibody molecule, or an anti-CEACAM (e.g., CEACAM-1, -3 or -5) antibody molecule) and/or anti-cancer agents, e.g., those listed in Table 1 and disclosed in the publications listed in Table 1.

In some embodiments, the cancer sample is classified as triple-positive for PD-L1/CD8/IFN-γ. This measurement can roughly be broken down into two thresholds: whether an individual cell is classified as positive, and whether the sample as a whole is classified as positive. First, one can measure, within an individual cell, the level of PD-L1, CD8, and/or IFN-γ. In some embodiments, a cell that is positive for one or more of these markers is a cell that has a higher level of the marker compared to a control cell or a reference value. For example, in some embodiments, a high level of PD-L1 in a given cell is a level higher than the level of PD-L1 in a corresponding non-cancerous tissue in the patient. As another example, in some embodiments, a high level of CD8 or IFN-γ in a given cell is a level of that protein typically seen in a TIL. Second, one can also measure the percentage of cells in the sample that are positive for PD-L1, CD8, and/or IFN-γ. (It is not necessary for a single cell to express all three markers.) In some embodiments, a triple positive sample is one that has a high percentage of cells, e.g., higher than a reference value or higher than a control sample, that are positive for these markers.

In other embodiments, one can measure the levels of PD-L1, CD8, and/or IFN-γ overall in the sample. In this case, a high level of CD8 or IFN-γ in the sample can be the level of that protein typically seen in a tumor infiltrated with TIL. Similarly, a high level of PD-L1 can be the level of that protein typically seen in a tumor sample, e.g., a tumor microenvironment.

The identification of subsets of patients that are triple-positive for PD-L1/CD8/IFN-γ, as shown in Example 1 herein, reveals certain sub-populations of patients that are likely to be especially responsive to PD-1 or PD-L1 antibody therapy. For instance, many IM-TN (immunomodulatory, triple negative) breast cancer patients are triple-positive for PD-L1/CD8/IFN-γ. IM-TN breast cancer is described in, e.g., Brian D. Lehmann et al., “Identification of human triple-negative breast cancer subtypes and preclinical models for selection of targeted therapies”, J Clin Invest. Jul. 1, 2011; 121(7): 2750-2767. Triple-negative breast cancers are those that do not express estrogen receptor (ER), progesterone receptor (PR) and Her2/neu. These cancers are difficult to treat because they are typically not responsive to agents that target ER, PR, and Her2/neu. Triple-negative breast cancers can be further subdivided into different classes, one of which is immunomodulatory. As described in Lehmann et al., IM-TN breast cancer is enriched for factors involved in immune cell processes, for example, one or more of immune cell signaling (e.g., TH1/TH2 pathway, NK cell pathway, B cell receptor signaling pathway, DC pathway, and T cell receptor signaling), cytokine signaling (e.g., cytokine pathway, IL-12 pathway, and IL-7 pathway), antigen processing and presentation, signaling through core immune signal transduction pathways (e.g., NFKB, TNF, and JAK/STAT signaling), genes involved in T-cell function, immune transcription, interferon (IFN) response and antigen processing. Accordingly, in some embodiments, the cancer treated is a cancer that is, or is determined to be, positive for one or more marker of IM-TN breast cancer, e.g., a factor that promotes one or more of immune cell signaling (e.g., TH1/TH2 pathway, NK cell pathway, B cell receptor signaling pathway, DC pathway, and T cell receptor signaling), cytokine signaling (e.g., cytokine pathway, IL-12 pathway, and IL-7 pathway), antigen processing and presentation, signaling through core immune signal transduction pathways (e.g., NFKB, TNF, and JAK/STAT signaling), genes involved in T-cell function, immune transcription, interferon (IFN) response and antigen processing.

As another example, it is shown herein that a subset of colon cancer patients having high MSI (microsatellite instability) is also triple-positive for PD-L1/CD8/IFN-γ. Accordingly, in some embodiments, a PD-1 or PD-L1 antibody, e.g., a PD-1 or PD-L1 antibody as described herein, (optionally in combination with one or more immunomodulators such as a LAG-3 antibody, TIM-3 antibody, or CEACAM (e.g., CEACAM-1, -3 and/or -5) antibody, and one or more anti-cancer agents, e.g., an anti-cancer agent described in Table 1 or in a publication in Table 1) is administered to a patient who has, or who is identified as having, colon cancer with high MSI, thereby treating the cancer. In some embodiments, a cell with high MSI is a cell having MSI at a level higher than a reference value or a control cell, e.g., a non-cancerous cell of the same tissue type as the cancer.

As another example, it is shown herein that a subset of gastric cancer patients having high MSI, and/or which is EBV+, is also triple-positive for PD-L1/CD8/IFN-γ. Accordingly, in some embodiments, a PD-1 or PD-L1 antibody, e.g., a PD-1 or PD-L1 antibody as described herein, (optionally in combination with one or more immunomodulators such as a LAG-3 antibody, TIM-3 antibody, or CEACAM (e.g., CEACAM-1, -3 and/or -5) antibody, and one or more anti-cancer agents, e.g., an anti-cancer agent described in Table 1 or in a publication in Table 1) is administered to a patient who has, or who is identified as having, gastric cancer with high MSI and/or EBV+, thereby treating the cancer. In some embodiments, a cell with high MSI is a cell having MSI at a level higher than a reference value or a control cell, e.g., a non-cancerous cell of the same tissue type as the cancer.

Additionally disclosed herein are methods of assaying a cancer for PD-L1, and then treating the cancer with a PD-1 or PD-L1 antibody. As described in Example 2 herein, a cancer sample can be assayed for PD-L1 protein levels or mRNA levels. A sample having levels of PD-L1 (protein or mRNA) higher than a reference value or a control cell (e.g., a non-cancerous cell) can be classified as PD-L1 positive. Accordingly, in some embodiments, a PD-1 or PD-L1 antibody, e.g., a PD-1 or PD-L1 antibody as described herein, (optionally in combination with one or more anti-cancer agents) is administered to a patient who has, or who is identified as having, a cancer that is PD-L1 positive. The cancer may be, e.g., non-small cell lung (NSCLC) adenocarcinoma (ACA), NSCLC squamous cell carcinoma (SCC), or hepatocellular carcinoma (HCC).

In some embodiments, the methods herein involve using a PD-1 or PD-L1 antibody, e.g., a PD-1 or PD-L1 antibody as described herein, e.g., as a monotherapy, for treating a cancer that is (or is identified as being) positive for PD-L1. In some embodiments, the cancer is colorectal cancer (e.g., MSI-high), gastric cancer (e.g., MSI-high and/or EBV+), NPC, cervical cancer, breast cancer (e.g., TN breast cancer), and ovarian cancer. In some embodiments, the cancer is NSCLC, melanoma, or HNSCC. In some embodiments, the PD-1 or PD-L1 antibody is administered at a dose of, e.g., 1, 3, 10, or 20 mg/kg.

Based on, e.g, Example 1 herein, it was found that certain gastric cancers that are triple-positive for PDL1/CD8/IFN-γ are also positive for PIK3CA. Accordingly, in some embodiments, a cancer can be treated with an anti-PD1 or anti-PD-L1 antibody molecule (optionally in combination with one or more immunomodulators, e.g., an anti-LAG3 antibody molecule, an anti-TIM-3 antibody molecule, or an anti-CEACAM (e.g., CEACAM-1, -3 and/or -5) antibody molecule) and an agent that inhibits PIK3CA. Exemplary agents in this category are described in Stein RC (September 2001). “Prospects for phosphoinositide 3-kinase inhibition as a cancer treatment”. Endocrine-related Cancer 8 (3): 237-48 and Marone R, Cmiljanovic V, Giese B, Wymann M P (January 2008). “Targeting phosphoinositide 3-kinase: moving towards therapy”. Biochimica et Biophysica Acta 1784 (1): 159-85.

Based on, e.g, Example 1 herein, CRC, e.g., a patient that has (or is identified as having) MSI-high CRC may be treated with a PD-1 or PD-L1 antibody, optionally in combination with a therapeutic that targets one or more of LAG-3, RNF43, and BRAF. For instance, these cancers may be treated with a PD-1 antibody, optionally in combination with one or more therapeutics that target one or more of LAG-3, PD-L1, RNF43, and BRAF. In embodiments, the one or more therapeutics include an immunomodulators such as an anti-LAG-3 antibody molecule, and an anti-cancer agent described in Table 1 or a publication listed in Table 1. LAG-3 inhibitors, e.g., antibodies, are described herein. RNF43 can be inhibited, e.g., with an antibody, small molecule (e.g., 2-(2′,3-dimethyl-[2,4′-bipyridin]-5-yl)-N-(5-(pyrazin-2-yl)pyridin-2-yl)acetamide (Compound A28)), siRNA, or a Rspo ligand or derivative thereof. BRAF inhibitors (e.g., vemurafenib or dabrafenib) are described herein.

Based on, e.g, Example 1 herein, a patient that has (or is identified as having) a squamous cell lung cancer may be treated with a PD-1 or PD-L1 antibody molecule in combination with a therapeutic that targets LAG-3, e.g., a LAG-3 antibody molecule, and optionally with one or more anti-cancer agents, e.g., an anti-cancer agent described in Table 1 or in a publication in Table 1.

In some embodiments, a subject that has (or is identified as having) a squamous cell lung cancer may be treated with a PD-1 or PD-L1 antibody, optionally in combination with a therapeutic that targets TIM-3, e.g., a TIM-3 antibody. TIM-3 inhibitors, e.g., antibodies, are described herein.

Based on, e.g, Example 1 herein, a patient that has (or is identified as having) a thyroid cancer may be treated with a PD-1 or PD-L1 antibody molecule, optionally in combination with a therapeutic that targets BRAF, and optionally in combination with one or more immunomodulators, e.g., an anti-LAG3 antibody molecule, an anti-TIM-3 antibody molecule, and an anti-PD-L1 antibody molecule. BRAF inhibitors (e.g., vemurafenib or dabrafenib) are described herein, e.g., in Table 1 and the publications listed in Table 1.

In some embodiments, the therapies here can be used to treat a patient that has (or is identified as having) a cancer associated with an infection, e.g., a viral or bacterial infection. Exemplary cancers include cervical cancer, anal cancer, HPV-associated head and neck squamous cell cancer, HPV-associated esophageal papillomas, HHV6-associated lymphomas, EBV-associated lymphomas (including Burkitt lymphoma), Gastric MALT lymphoma, other infection-associated MALT lymphomas, HCC, Kaposi's sarcoma.

### Dosages and Administration

Dosages and therapeutic regimens of the agents described herein can be determined by a skilled artisan.

In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

In one embodiment, the anti-PD-1 antibody molecule, e.g., Nivolumab, is administered intravenously at a dose from about 1 mg/kg to 3 mg/kg, e.g., about 1 mg/kg, 2 mg/kg or 3 mg/kg, every two weeks. In one embodiment, the anti-PD-1 antibody molecule, e.g., Nivolumab or Pembrolizumab, is administered intravenously at a dose of about 2 mg/kg at 3-week intervals. In one embodiment, Nivolumab is administered in an amount from about 1 mg/kg to 5 mg/kg, e.g., 3 mg/kg, and may be administered over a period of 60 minutes, once a week to once every 2, 3 or 4 weeks. In one embodiment, Pembrolizumab is administered in an amount from about 1 mg/kg to 5 mg/kg, e.g., 3 mg/kg, and may be administered over a period of 30 minutes, once a week to once every 2, 3 or 4 weeks.

In one embodiment, the anti-PD-1 antibody molecule, e.g., Pembrolizumab, is administered intravenously at a dose from about 50 mg to 500 mg, e.g., about 50 mg, 100 mg, 150 mg, 200 mg, 250 mg, 300 mg, 350 mg, 400 mg, 450 mg, or 500 mg, every two weeks. In one embodiment, the anti-PD-1 antibody molecule, e.g., Pembrolizumab, is administered intravenously at a dose of about 50 mg to 500 mg, e.g., 100 mg to 400 mg, 150 mg to 250 mg, or 200 mg to 300 mg, e.g., 200 mg, and may be administered once a week or once every 2, 3 or 4 weeks. In one embodiment, Pembrolizumab is administered at a dose of about 200 mg at 3-week intervals.

In certain embodiments, the anti-PD-L1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, 4, 5 or 6 weeks. In one embodiment, the anti-PD-L1 antibody molecule is administered at a dose from about 10 to 20 mg/kg every other week.

In one embodiment, the anti-PD-L1 antibody molecule, e.g., Pidilizumab, is administered intravenously at a dose from about 1 mg/kg to 3 mg/kg, e.g., about 1 mg/kg, 1.5 mg/kg, 2 mg/kg, 2.5 mg/kg, or 3 mg/kg, once every two weeks or once every four weeks. In one embodiment, Pidilizumab is administered in an amount from about 1 mg/kg to 5 mg/kg, e.g., 3 mg/kg, and may be administered over a period of 60 minutes, once a week to once every 2, 3, 4, 5 or 6 weeks. In one embodiment, the anti-PD-L1 antibody molecule, e.g., Pidilizumab, is administered intravenously at a dose of about 3 mg/kg at 4-week intervals. The combination therapies described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

The methods and compositions described herein can be used in combination with further agents or therapeutic modalities. The combination therapies can be administered simultaneously or sequentially in any order. Any combination and sequence of the anti-PD-1 or PD-L1 antibody molecules and other therapeutic agents, procedures or modalities (e.g., as described herein) can be used. The combination therapies can be administered during periods of active disorder, or during a period of remission or less active disease. The combination therapies can be administered before the other treatment, concurrently with the treatment, post-treatment, or during remission of the disorder.

In certain embodiments, the methods and compositions described herein are administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation (e.g., gamma irradiation). In other embodiments, the additional therapy is surgery or radiation, or a combination thereof. In other embodiments, the additional therapy is a therapy targeting an mTOR pathway, an HSP90 inhibitor, or a tubulin inhibitor.

Alternatively, or in combination with the aforesaid combinations, the methods and compositions described herein can be administered in combination with one or more of: a vaccine, e.g., a therapeutic cancer vaccine; or other forms of cellular immunotherapy.

In another embodiment, the combination therapy is used in combination with one, two or all of oxaliplatin, leucovorin or 5-FU (e.g., a FOLFOX co-treatment). Alternatively or in combination, combination further includes a VEGF inhibitor (e.g., a VEGF inhibitor as disclosed herein). In some embodiments, the cancer treated with the combination is chosen from a melanoma, a colorectal cancer, a non-small cell lung cancer, an ovarian cancer, a breast cancer, a prostate cancer, a pancreatic cancer, a hematological malignancy or a renal cell carcinoma. The cancer may be at an early, intermediate or late stage.

In other embodiments, the combination therapy is administered with a tyrosine kinase inhibitor (e.g., axitinib) to treat renal cell carcinoma and other solid tumors.

In other embodiments, the combination therapy is administered with a 4-1BB receptor targeting agent (e.g., an antibody that stimulates signaling through 4-1BB (CD-137), e.g., PF-2566). In one embodiment, the combination therapy is administered in combination with a tyrosine kinase inhibitor (e.g., axitinib) and a 4-1BB receptor targeting agent.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 shows exemplary cancers having relatively high proportions of patients that are triple-positive for PD-L1/CD8/IFN-γ.

FIG. 2 shows exemplary ER+ breast cancer and pancreatic cancer having relatively low proportions for patients that are triple positive for PD-L1/CD8/IFN-γ.

FIG. 3 shows the proportion of exemplary breast cancer patients that are triple positive for PD-L1/CD8/IFN-γ.

FIG. 4 shows the proportion of exemplary colon cancer patients that are triple positive for PD-L1/CD8/IFN-γ.

### BRIEF DESCRIPTION OF THE TABLE

Table 1 is a summary of selected therapeutic agents that can be administered in combination with the immunomodulators (e.g., one or more of: an activator of a costimulatory molecule and/or an inhibitor of an immune checkpoint molecule) described herein. Table 1 provides from left to right the following: the Compound Designation of the second therapeutic agent, the Compound structure, and Patent publication(s) disclosing the Compound.

### DETAILED DESCRIPTION

Methods and compositions are disclosed, which comprise an immunomodulator (e.g., one or more of: an activator of a costimulatory molecule and/or an inhibitor of an immune checkpoint molecule) in combination with a second therapeutic agent chosen from one or more of the agents listed in Table 1. Immune therapy alone can be effective in a number of indications (e.g., melanoma). However, for most patients, it is not a cure. In one embodiment, an inhibitor of an immune checkpoint molecule (e.g., one or more of inhibitors to PD-1, PD-L1, LAG-3, TIM-3 or CTLA-4) can be combined with a second therapeutic agent chosen from one or more of listed in Table 1 (e.g., chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor); 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor. The combinations described herein can provide a beneficial effect, e.g., in the treatment of a cancer, such as an enhanced anti-cancer effect, reduced toxicity and/or reduced side effects. When administered in combination, the immunomodualtor, the additional agent (e.g., second or third agent), or all, can be administered in an amount or dose that is higher, lower or the same than the amount or dosage of each agent used individually, e.g., as a monotherapy. For example, the immunomodulator, the second therapeutic agent, or both, can be administered at a lower dosage than would be required to achieve the same therapeutic effect compared to a monotherapy dose. In certain embodiments, the administered amount or dosage of the immunomodulator, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the immunomodulator, the additional agent (e.g., second or third agent), or all, that results in a desired effect (e.g., treatment of cancer) is lower (e.g., at least 20%, at least 30%, at least 40%, or at least 50% lower). The term “activation” or “activator” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “inhibition” or “inhibitor” includes a reduction in a certain parameter, e.g., an activity, of a given molecule, e.g., an immune checkpoint inhibitor. For example, inhibition of an activity, e.g., an activity of, e.g., PD-1, PD-L1, PKC, HSP90, PI3K, mTOR, cytochrome P450, aromatase, aldosterone synthase, SMO, PRLR, Wnt, CDK4/6, FGFR2, FGFR4, M-CSF, c-KIT, Flt3, PKC, VEGFR-2, PDGFRbeta, Raf kinase C, ALK, IGF-1R, P-Glycoprotein 1, VEGFR, IDH, BCL-ABL, cRAF, ERK1/2, or CSF-1R, of at least 5%, 10%, 20%, 30%, 40% or more is included by this term. Thus, inhibition need not be 100%.

The term “Programmed Death 1” or “PD-1” includes all isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

The term or “PD-Ligand 1” or “PD-L1” includes all isoforms, mammalian, e.g., human PD-1, species homologs of human PD-L1, and analogs comprising at least one common epitope with PD-L1. The amino acid sequence of PD-L1, e.g., human PD-L1, is known in the art, e.g., Dong H, et al. (1999) Nat. Med. 5 (12):1365-1369; Freeman G et al. (2000) J. Exp. Med. 192 (7):1027-1034.

The term “Lymphocyte Activation Gene-3” or “LAG-3” includes all isoforms, mammalian, e.g., human LAG-3, species homologs of human LAG-3, and analogs comprising at least one common epitope with LAG-3. The amino acid and nucleotide sequences of LAG-3, e.g., human LAG-3, is known in the art, e.g., Triebel et al. (1990) J. Exp. Med. 171:1393-1405.

The term “T-cell Immunoglobulin, Mucin Domain-3” or “TIM-3” includes all isoforms, mammalian, e.g., human TIM-3, species homologs of human LAG-3, and analogs comprising at least one common epitope with TIM-3. The amino acid and nucleotide sequendces of TIM-3, e.g., human TIM-3, is known in the art, e.g., McIntire J et al. (2001) Nat Immunol. 2(12):1109-16; Monney L. et al. Nature (2002) 415(6871):536-41. TIM-3 has a role in regulating immunity and tolerance in vivo (see Hastings et al., Eur J Immunol. 2009 September; 39(9):2492-501).

The term “Carcinoembryonic Antigen-related Cell Adhesion Molecule” or “CEACAM” includes all family members (e.g., CEACAM-1, CEACAM-3, or CEACAM-5), isoforms, mammalian, e.g., human CEACAM, species homologs of human CEACAM, and analogs comprising at least one common epitope with CEACAM. The amino acid sequence of CEACAM, e.g., human CEACAM, is known in the art, e.g., Hinoda et al. (1988) Proc. Natl. Acad. Sci. U.S.A. 85 (18), 6959-6963; Zimmermann W. et al. (1987) Proc. Natl. Acad. Sci. U.S.A. 84 (9), 2960-2964; Thompson J. et al. (1989) Biochem. Biophys. Res. Commun. 158 (3), 996-1004.

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Antibody Molecules

In one embodiment, the antibody molecule binds to a mammalian, e.g., human, checkpoint molecule, e.g., PD-1, PD-L1, LAG-3, CEACAM (e.g., CEACAM-1, -3 and/or -5), or TIM-3. For example, the antibody molecule binds specifically to an epitope, e.g., linear or conformational epitope, (e.g., an epitope as described herein) on PD-1, PD-L1, LAG-3, (e.g., CEACAM-1, -3 and/or -5), or TIM-3.

As used herein, the term “antibody molecule” refers to a protein, e.g., an immunoglobulin chain or fragment thereof, comprising at least one immunoglobulin variable domain sequence.

The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule,

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the first epitope is located on PD-1 and the second epitope is located on a TIM-3, LAG-3, CEACAM (e.g., CEACAM-1 and/or CEACAM-5), PD-L1, or PD-L2.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

An antibody molecules may be conjugated to another molecular entity, typically a label or a therapeutic (e.g., a cytotoxic or cytostatic) agent or moiety. Radioactive isotopes can be used in diagnostic or therapeutic applications. Radioactive isotopes that can be coupled to the anti-PSMA antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In) technetium (⁹⁹mTc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Combination Therapies

The combination therapies (e.g., methods and compositions described herein) can include an immunomodulator (e.g., one or more of: an activator of a costimulatory molecule or an inhibitor of an inhibitory molecule) and a second therapeutic agent, e.g., a second therapeutic agent chosen from one or more of the agents listed in Table 1.

By “combination” or “in combination with,” it is not intended to imply that the therapy or the therapeutic agents must be administered at the same time and/or formulated for delivery together (e.g., in the same composition), although these methods of delivery are within the scope described herein. The immunomodulator and the second therapeutic agent can be administered concurrently with, prior to, or subsequent to, one or more other additional therapies or therapeutic agents. The agents in the combination can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In some embodiments, a combination includes a formulation of the immunomodulator and the second therapeutic agent, with or without instructions for combined use or to combination products. The combined compounds can be manufactured and/or formulated by the same or different manufacturers. The combination partners may thus be entirely separate pharmaceutical dosage forms or pharmaceutical compositions that are also sold independently of each other. In some embodiments, instructions for their combined use are provided: (i) prior to release to physicians (e.g. in the case of a “kit of part” comprising the compound of the disclosure and the other therapeutic agent); (ii) by the physicians themselves (or under the guidance of a physician) shortly before administration; (iii) the patient themselves by a physician or medical staff.

### Immunomodulators

The combination therapies disclosed herein can include an inhibitor of an inhibitory molecule of an immune checkpoint molecule. The term “immune checkpoints” refers to a group of molecules on the cell surface of CD4 and CD8 T cells. These molecules can effectively serve as “brakes” to down-modulate or inhibit an anti-tumor immune response. Inhibition of an inhibitory molecule can be performed by inhibition at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand, or an antibody or antigen-binding fragment thereof, that binds to the inhibitory molecule.

Immune checkpoint molecules useful in the methods and compositions of the present invention include, but are not limited to, Programmed Death 1 (PD-1), PD-L1, PD-L2, Cytotoxic T-Lymphocyte Antigen 4 (CTLA-4), TIM-3, CEACAM (e.g., CEACAM-1, -3 and/or -5), VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGFR (e.g., TGFR beta), B7-H1, B7-H4 (VTCN1), OX-40, CD137, CD40, and LAGS. In certain embodiments, the immunomodulator is an inhibitor of an immune checkpoint molecule (e.g., an inhibitor of PD-1, PD-L1, LAG-3, TIM-3, CEACAM (e.g., CEACAM-1, -3 and/or -5) or CTLA-4, or any combination thereof). In certain embodiments, the anti-PD-1 molecules described herein are administered in combination with one or more other inhibitors of PD-1, PD-L1 and/or PD-L2 known in the art. The antagonist may be an antibody, an antigen binding fragment thereof, an immunoadhesin, a fusion protein, or oligopeptide.

In other embodiments, the PD-1 inhibitor is an anti-PD-1 antibody chosen from Nivolumab, Pembrolizumab or Pidilizumab.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449, EP2161336 and WO2006/121168. In one embodiment, the inhibitor of PD-1 is Nivolumab, and having a sequence disclosed herein (or a sequence substantially identical or similar thereto, e.g., a sequence at least 85%, 90%, 95% identical or higher to the sequence specified).

The heavy and light chain amino acid sequences of Nivolumab are as follows:

Heavy chain
(SEQ ID NO: 2)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLEPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light chain
(SEQ ID NO: 3)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, the anti-PD-1 antibody is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335. In one embodiment, the inhibitor of PD-1 is Pembrolizumab disclosed in, e.g., U.S. Pat. No. 8,354,509 and WO 2009/114335, and having a sequence disclosed herein (or a sequence substantially identical or similar thereto, e.g., a sequence at least 85%, 90%, 95% identical or higher to the sequence specified).

The heavy and light chain amino acid sequences of Pembrolizumab are as follows:

| Heavy chain | |
|---|---|
| (SEQ ID NO: 4) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| RVVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Ligh chain | |
| (SEQ ID NO: 5) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611.

Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224.

### Exemplary PD-L1 or PD-L2 Inhibitors

In some embodiments, the PD-L1 inhibitor is an antibody molecule. In some embodiments, the anti-PD-L1 inhibitor is chosen from YW243.55.570, MPDL3280A, MEDI-4736, MSB-0010718C, or MDX-1105.

In some embodiments, the anti-PD-L1 antibody is MSB0010718C. MSB0010718C (also referred to as A09-246-2; Merck Serono) is a monoclonal antibody that binds to PD-L1. Pembrolizumab and other humanized anti-PD-L1 antibodies are disclosed in WO2013/079174, and having a sequence disclosed herein (or a sequence substantially identical or similar thereto, e.g., a sequence at least 85%, 90%, 95% identical or higher to the sequence specified).

The heavy and light chain amino acid sequences of MSB0010718C include at least the following:

Heavy chain (SEQ ID NO: 24 as disclosed in
WO2013/079174)
(SEQ ID NO: 6)
EVQLLESGGGLVQPGGSLRLSCAASGFTFSSYIMMWVRQAPGKGLEWVSS
TYPSGGITFYADKGRETISRDNSKNTLYLQMNSLRAEDTAVYYCARIKLG
TVTTVDYWGQGTLVTVSS
Light chain (SEQ ID NO: 25 as disclosed in
WO2013/079174)
(SEQ ID NO: 7)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
EGTGTKVTVL

In one embodiment, the PD-L1 inhibitor is YW243.55.S70. The YW243.55.S70 antibody is an anti-PD-L1 described in WO 2010/077634 (heavy and light chain variable region sequences shown in SEQ ID Nos. 20 and 21, respectively, of WO 2010/077634), and having a sequence disclosed therein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in WO2007/005874, and having a sequence disclosed therein (or a sequence substantially identical or similar thereto, e.g., a sequence at least 85%, 90%, 95% identical or higher to the sequence specified).

In one embodiment, the PD-L1 inhibitor is MDPL3280A (Genentech/Roche). MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906. Other anti-PD-L1 binding agents include YW243.55.570 (heavy and light chain variable regions are shown in SEQ ID NOs 20 and 21 in WO2010/077634) and MDX-1105 (also referred to as BMS-936559, and, e.g., anti-PD-L1 binding agents disclosed in WO2007/005874).

In other embodiments, the PD-L2 inhibitor is AMP-224. AMP-224 is a PD-L2 Fc fusion soluble receptor that blocks the interaction between PD-1 and B7-H1 (B7-DCIg; Amplimmune; e.g., disclosed in WO2010/027827 and WO2011/066342).

### Exemplary TIM-3 Inhibitors

In one embodiment, a combination described herein includes a TIM-3 inhibitor. In some embodiments, the combination is used to treat a cancer, e.g., a cancer described herein, e.g., a solid tumor or a hematologic malignancy.

Exemplary anti-TIM-3 antibodies are disclosed in U.S. Pat. No. 8,552,156, WO 2011/155607, EP 2581113 and U.S. Publication No.: 2014/044728.

### Exemplary LAG-3 Inhibitors

In one embodiment, a combination described herein includes a LAG-3 inhibitor. In some embodiments, the combination is used to treat a cancer, e.g., a cancer described herein, e.g., a solid tumor or a hematologic malignancy.

In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218.

### Exemplary CTLA-4 Inhibitors

In one embodiment, a combination described herein includes a CTLA-4 inhibitor. In some embodiments, the combination is used to treat a cancer, e.g., a cancer described herein, e.g., a solid tumor or a hematologic malignancy.

Exemplary anti-CTLA-4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

In one embodiment, the combination includes an anti-PD-1 antibody molecule, e.g., as described herein, and an anti-CTLA-4 antibody, e.g., ipilimumab. Exemplary doses that can be use include a dose of anti-PD-1 antibody molecule of about 1 to 10 mg/kg, e.g., 3 mg/kg, and a dose of an anti-CTLA-4 antibody, e.g., ipilimumab, of about 3 mg/kg. In one embodiment, the anti-PD-1 antibody molecule is administered after treatment, e.g., after treatment of a melanoma, with an anti-CTLA-4 antibody (e.g., ipilimumab) with or without a BRAF inhibitor (e.g., vemurafenib or dabrafenib).

Other exemplary anti-CTLA-4 antibodies are disclosed, e.g., in U.S. Pat. No. 5,811,097.

In one embodiment, the inhibitor is a soluble ligand (e.g., a CTLA-4-Ig), or an antibody or antibody fragment that binds to PD-L1, PD-L2 or CTLA-4. For example, the anti-PD-1 antibody molecule can be administered in combination with an anti-CTLA-4 antibody, e.g., ipilimumab, for example, to treat a cancer (e.g., a cancer chosen from: a melanoma, e.g., a metastatic melanoma; a lung cancer, e.g., a non-small cell lung carcinoma; or a prostate cancer).

### Additional Combinations of Inhibitors

In certain embodiments, the anti-PD-1 molecules described herein are administered in combination with one or more other inhibitors of PD-1, PD-L1 and/or PD-L2, e.g., as described herein. The antagonist may be an antibody, an antigen binding fragment thereof, an immunoadhesin, a fusion protein, or oligopeptide.

In one embodiment, the anti-PD-1 or PD-L1 antibody molecule is administered in combination with an anti-LAG-3 antibody or an antigen-binding fragment thereof. In another embodiment, the anti-PD-1 or PD-L1 antibody molecule is administered in combination with an anti-TIM-3 antibody or antigen-binding fragment thereof. In yet other embodiments, the anti-PD-1 or PD-L1 antibody molecule is administered in combination with an anti-LAG-3 antibody and an anti-TIM-3 antibody, or antigen-binding fragments thereof. The combination of antibodies recited herein can be administered separately, e.g., as separate antibodies, or linked, e.g., as a bispecific or trispecific antibody molecule. In one embodiment, a bispecific antibody that includes an anti-PD-1 or PD-L1 antibody molecule and an anti-TIM-3 or anti-LAG-3 antibody, or antigen-binding fragment thereof, is administered. In certain embodiments, the combination of antibodies recited herein is used to treat a cancer, e.g., a cancer as described herein (e.g., a solid tumor). The efficacy of the aforesaid combinations can be tested in animal models known in the art. For example, the animal models to test the synergistic effect of anti-PD-1 and anti-LAG-3 are described, e.g., in Woo et al. (2012) Cancer Res. 72(4):917-27).

In another embodiment, the anti-PD-1 or PD-L1 antibody molecule is administered in combination with an inhibitor of CEACAM (e.g., CEACAM-1, -3 and/or -5). In one embodiment, the inhibitor of CEACAM (e.g., CEACAM-1, -3 and/or -5) is an anti-CEACAM antibody molecule. Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

Accordingly, in some embodiments, the anti-PD-1 antibody molecule is administered in combination with a CEACAM inhibitor (e.g., CEACAM-1, CEACAM-3, and/or CEACAM-5 inhibitor). In one embodiment, the inhibitor of CEACAM is an anti-CEACAM antibody molecule. In one embodiment, the anti-PD-1 antibody molecule is administered in combination with a CEACAM-1 inhibitor, e.g., an anti-CEACAM-1 antibody molecule. In another embodiment, the anti-PD-1 antibody molecule is administered in combination with a CEACAM-3 inhibitor, e.g., an anti-CEACAM-3 antibody molecule. In another embodiment, the anti-PD-1 antibody molecule is administered in combination with a CEACAM-5 inhibitor, e.g., an anti-CEACAM-5 antibody molecule. Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

In another embodiment, the anti-PD-1 or PD-L1 antibody molecule is administered in combination with an anti-LAG-3 antibody or an antigen-binding fragment thereof. In another embodiment, the anti-PD-1 or PD-L1 antibody molecule is administered in combination with an anti-TIM-3 antibody or antigen-binding fragment thereof. In yet other embodiments, the anti-PD-1 or PD-L1 antibody molecule is administered in combination with an anti-LAG-3 antibody and an anti-TIM-3 antibody, or antigen-binding fragments thereof. The combination of antibodies recited herein can be administered separately, e.g., as separate antibodies, or linked, e.g., as a bispecific or trispecific antibody molecule. In one embodiment, a bispecific antibody that includes an anti-PD-1 or PD-L1 antibody molecule and an anti-TIM-3 or anti-LAG-3 antibody, or antigen-binding fragment thereof, is administered. In certain embodiments, the combination of antibodies recited herein is used to treat a cancer, e.g., a cancer as described herein (e.g., a solid tumor). The efficacy of the aforesaid combinations can be tested in animal models known in the art. For example, the animal models to test the synergistic effect of anti-PD-1 and anti-LAG-3 are described, e.g., in Woo et al. (2012) Cancer Res. 72(4):917-27).

### Costimulatory Modulators

In certain embodiments, the combination therapies disclosed herein include a modulator of a costimulatory molecule.

In one embodiment, the costimulatory modulator, e.g., agonist, of a costimulatory molecule is chosen from an agonist (e.g., an agonistic antibody or antigen-binding fragment thereof, or soluble fusion) of an MHC class I molecule, a TNF receptor protein, an Immunoglobulin-like proteins, a cytokine receptor, an integrin, a signaling lymphocytic activation molecules (SLAM proteins), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), SLAM7, BLAME (SLAMF8), SELPLG (CD162), LTBR, LAT, GADS, SLP-76, PAG/Cbp, CD19a, and a ligand that specifically binds with CD83.

In another embodiment, the combination therapies disclosed herein include a costimulatory molecule, e.g., an agonist associated with a positive signal that includes a costimulatory domain of CD28, CD27, ICOS and GITR.

### Exemplary GITR Agonist

In one embodiment, a combination described herein includes a GITR agonist. In some embodiments, the combination is used to treat a cancer, e.g., a cancer described herein, e.g., a solid tumor or a hematologic malignancy.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. No. 7,812,135, U.S. Pat. No. 8,388,967, U.S. Pat. No. 8,591,886, European Patent No.: EP 1866339, PCT Publication No.: WO 2011/028683, U.S. Pat. No. 8,709,424, PCT Publication No.: WO 2013/039954, U.S. Publication No.: US2014/0072566, PCT Publication No.: WO2015/026684, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, U.S. Pat. No. 6,689,607, PCT Publication No.: WO2006/083289, PCT Publication No.: WO 2005/115451, U.S. Pat. No. 7,618,632, PCT Publication No.: WO 2011/051726, PCT Publication No.: WO2004/060319, and PCT Publication No.: WO2014/012479.

In one embodiment, the GITR agonist is used in combination with a PD-1 inhibitor, e.g., as described in WO2015/026684.

In another embodiment, the GITR agonist is used in combination with a TLR agonist, e.g., as described in WO2004/060319, and International Publication No.: WO2014/012479.

### Additional Combinations

In another embodiment, the combination therapies include a modified T-cell, e.g., in combination with an adoptive T-cell immunotherapy using chimeric antigen receptor (CAR) T cells (e.g., as described by John L B, et al. (2013) Clin. Cancer Res. 19(20): 5636-46).

In other embodiments, the combination therapies disclosed herein can also include a cytokine, e.g., interleukin-21 or interleukin-2. In certain embodiments, the combination described herein is used to treat a cancer, e.g., a cancer as described herein (e.g., a solid tumor or melanoma).

Exemplary immunomodulators that can be used in the combination therapies include, but are not limited to, e.g., afutuzumab (available from ROCHE®); pegfilgrastim (NEULASTA®); lenalidomide (CC-5013, REVLIMID®); thalidomide (THALOMID®), actimid (CC4047); and cytokines, e.g., IL-21 or IRX-2 (mixture of human cytokines including interleukin 1, interleukin 2, and interferon γ, CAS 951209-71-5, available from IRX Therapeutics).

In other embodiments, the combination therapies can be administered to a subject in conjunction with (e.g., before, simultaneously or following) one or more of: bone marrow transplantation, T cell ablative therapy using chemotherapy agents such as, fludarabine, external-beam radiation therapy (XRT), cyclophosphamide, and/or antibodies such as OKT3 or CAMPATH. In one embodiment, the anti-PD-1 or PD-L1 antibody molecules are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive the anti-PD-1 or PD-L1 antibody molecules. In an additional embodiment, the anti-PD-1 or PD-L1 antibody molecules are administered before or following surgery.

Another example of a further combination therapy includes decarbazine for the treatment of melanoma. Without being bound by theory, the combined use of PD-1 blockade and chemotherapy is believed to be facilitated by cell death, that is a consequence of the cytotoxic action of most chemotherapeutic compounds, which can result in increased levels of tumor antigen in the antigen presentation pathway. Other combination therapies that may result in synergy with PD-1 blockade through cell death are radiation, surgery, and hormone deprivation. Each of these protocols creates a source of tumor antigen in the host. Angiogenesis inhibitors may also be combined with PD-1 blockade. Inhibition of angiogenesis leads to tumor cell death which may feed tumor antigen into host antigen presentation pathways.

Combination therapies can also be used in combination with bispecific antibodies. Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would by augmented by the use of PD-1 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Antibodies or antigen-binding fragments thereof to each of these entities may be used in combination with anti-PD-1 to counteract the effects of the immunosuppressive agent and favor tumor immune responses by the host.

Other antibodies which may be used to activate host immune responsiveness can be used in combination with the combination therapies described herein. These include molecules on the surface of dendritic cells which activate DC function and antigen presentation. Anti-CD40 antibodies are able to substitute effectively for T cell helper activity (Ridge, J. et al. (1998) Nature 393: 474-478) and can be used in conjunction with PD-1 antibodies (Ito, N. et al. (2000) Immunobiology 201 (5) 527-40). Antibodies to T cell costimulatory molecules such as CTLA-4 (e.g., U.S. Pat. No. 5,811,097), OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) may also provide for increased levels of T cell activation.

In all of the methods described herein, PD-1 blockade can be combined with other forms of immunotherapy such as cytokine treatment (e.g., interferons, GM-CSF, G-CSF, IL-2, IL-21), or bispecific antibody therapy, which provides for enhanced presentation of tumor antigens (see e.g., Holliger (1993) Proc. Natl. Acad. Sci. USA 90:6444-6448; Poljak (1994) Structure 2:1121-1123).

The combination therapies disclosed herein can be further combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF.

PD-1 blockade can be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci. U.S.A. 90: 3539-43).

PD-1 blockade can be used in conjunction with a collection of recombinant proteins and/or peptides expressed in a tumor in order to generate an immune response to these proteins.

These proteins are normally viewed by the immune system as self antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. (1994) Science 266: 2011-2013). These somatic tissues may be protected from immune attack by various means. Tumor antigen may also be “neo-antigens” expressed in cancer cells because of somatic mutations that alter protein sequence or create fusion proteins between two unrelated sequences (ie. bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with PD-1 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with PD-1 blockade to activate more potent anti-tumor responses.

### Second Therapeutic Agents

The second therapeutic agent can be chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) a transduction modulator and/or apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor; e.g., chosen from one or more of the agents listed in Table 1.

TABLE 1
Selected therapeutic agents that can be administered in combination with the
immunomodulators, e.g., as a single agent or in combination with other immunomodulators
described herein. Each publication listed in this Table is herein incorporated by reference in its
entirety, including all structural formulae therein.

| | Generic | | Patents/Patent |
|---|---|---|---|
| Compound | Name | | Application |
| No. | Tradename | Compound Structure | Publications |
| A | Sotrastaurin | 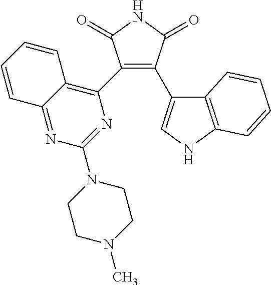 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| B | | 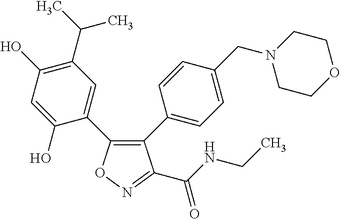 [SMILES: CCNC(=O)c1noc(-c2cc(C(C)C)c(O)cc2O)c1-c1ccc(CN2CCOCC2)cc1] | WO 2010/060937 WO 2004/072051 EP 1611112 U.S. Pat. No. 8,450,310 |
| C | Dactolisib | 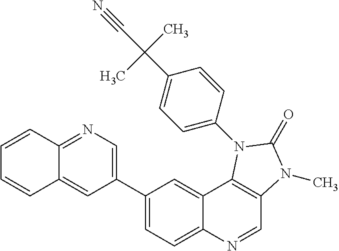 [SMILES: Cn1c(=O)n(-c2ccc(C(C)(C)C#N)cc2)c2c3cc(-c4cnc5ccccc5c4)ccc3ncc21] | WO 2006/122806 |
| D | | CYP17 inhibitor | WO 2010/149755 |
| | | | U.S. Pat. No. 8,263,635 B2 |
| | | | EP 2445903 B1 |
| E | Deferasirox EXJADE ® | 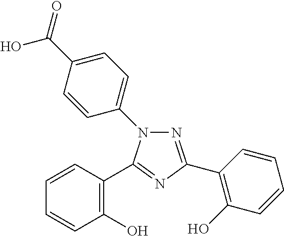 [SMILES: O=C(O)c1ccc(-n2nc(-c3ccccc3O)nc2-c2ccccc2O)cc1] | WO 1997/049395 |
| F | Letrozole FEMARA ® | 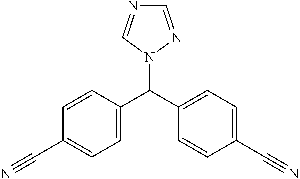 [SMILES: N#Cc1ccc(C(c2ccc(C#N)cc2)n2cncn2)cc1] | U.S. Pat. No. 4,978,672 |
| G | | 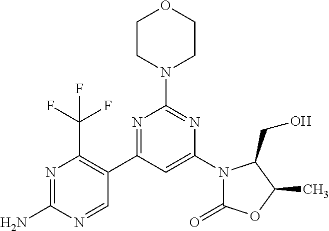 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| H | | 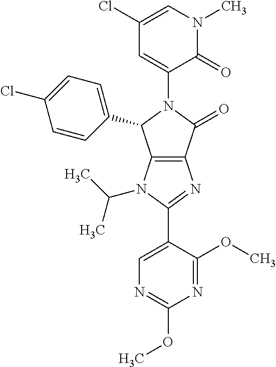 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| I | Imatinib mesylate GLEEVEC ® | 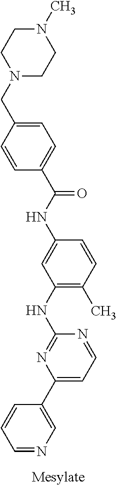 [SMILES: Cc1ccc(NC(=O)c2ccc(CN3CCN(C)CC3)cc2)cc1Nc1nccc(-c2cccnc2)n1] | WO 1999/003854 |
| J | Osilodrostat | 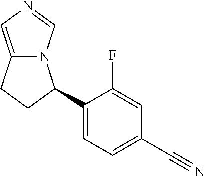 [SMILES: N#Cc1ccc([C@H]2CCc3cncn32)c(F)c1] | WO 2007/024945 |
| K | Sonidegib phosphate | 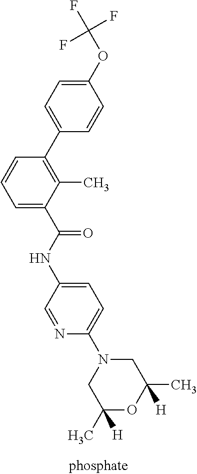 [SMILES: Cc1c(C(=O)Nc2ccc(N3C[C@@H](C)O[C@@H](C)C3)nc2)cccc1-c1ccc(OC(F)(F)F)cc1] | WO 2007/131201 EP 2021328 U.S. Pat. No. 8,178,563 |
| L | | 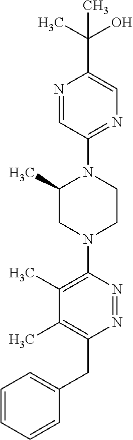 [SMILES: Cc1c(Cc2ccccc2)nnc(N2CCN(c3cnc(C(C)(C)O)cn3)[C@H](C)C2)c1C] | WO 2010/007120 |
| M | | Human monoclonal antibody to PRLR | U.S. Pat. No. 7,867,493 |
| N | | 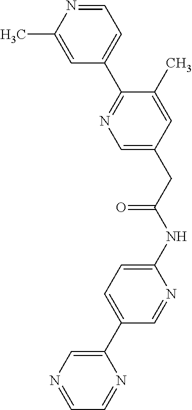 [SMILES: Cc1cc(-c2ncc(CC(=O)Nc3ccc(-c4cnccn4)cn3)cc2C)ccn1] | WO 2010/101849 |
| O | | 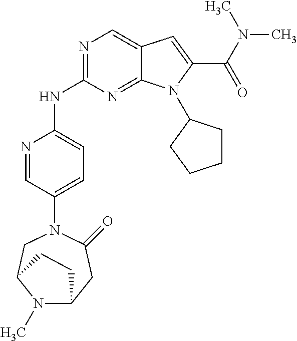 [SMILES: CN(C)C(=O)c1cc2cnc(Nc3ccc(N4C[C@H]5CC[C@@H](CC4=O)N5C)cn3)nc2n1C1CCCC1] | WO 2011/101409 |
| P | | Antibody Drug Conjugate (ADC) | WO 2014/160160 |
| | | | Ab: 12425 (see Table 1, |
| | | | paragraph [00191]) |
| | | | Linker: SMCC (see |
| | | | paragraph [00117] |
| | | | Payload: DM1 (see |
| | | | paragraph [00111] |
| | | | See also Claim 29 |
| Q | | Monoclonal antibody or Fab to M-CSF | WO 2004/045532 |
| R | Midostaurin | 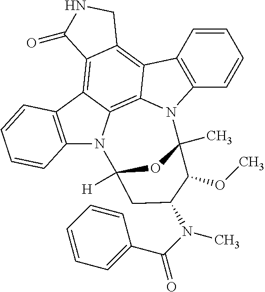 | WO 2003/037347 EP 1441737 US 2012/252785 |
| S | | 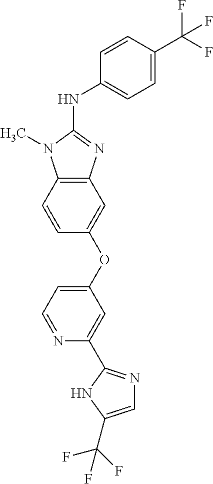 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| T | Pasireotide diaspartate SIGNIFOR ® | 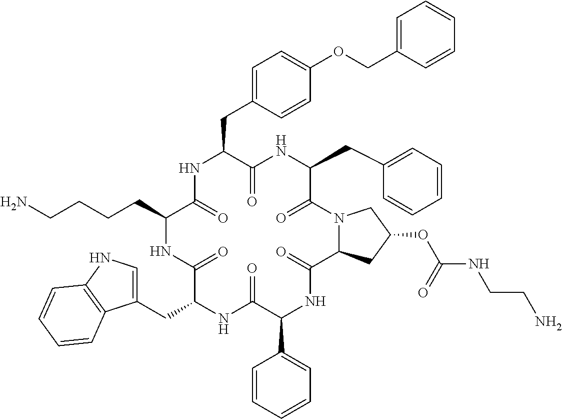 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O] | WO2002/010192 U.S. Pat. No. 7,473,761 |
| U | Dovitinib | 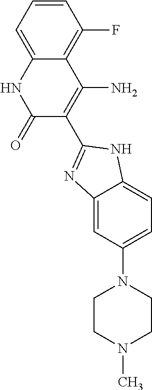 [SMILES: CN1CCN(c2ccc3[nH]c(-c4c(N)c5c(F)cccc5[nH]c4=O)nc3c2)CC1] | WO 2009/115562 U.S. Pat. No. 8,563,556 |
| V | | 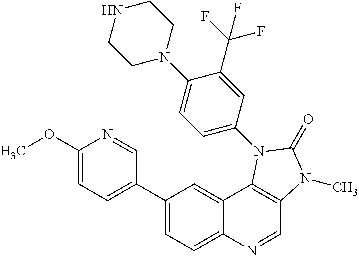 | WO 2006/122806 |
| W | | 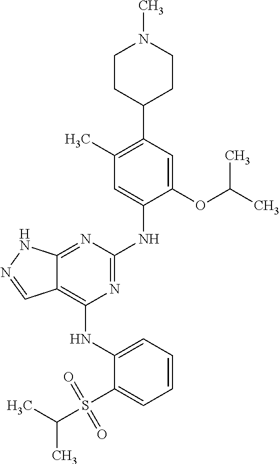 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| X | | 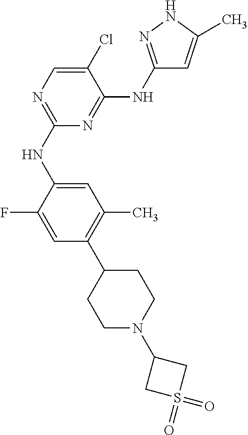 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| Y | | 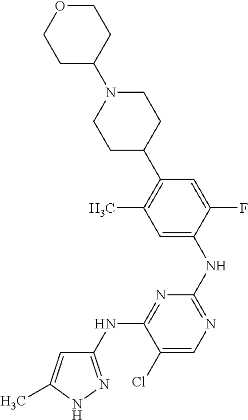 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| Z | | 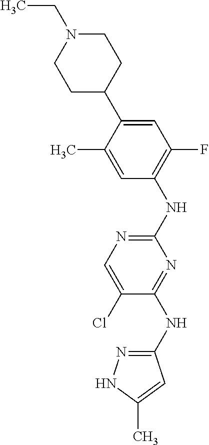 [SMILES: CCN1CCC(c2cc(F)c(Nc3ncc(Cl)c(Nc4cc(C)[nH]n4)n3)cc2C)CC1] | WO 2010/002655 |
| AA | Valspodar AMDRAY ™ | 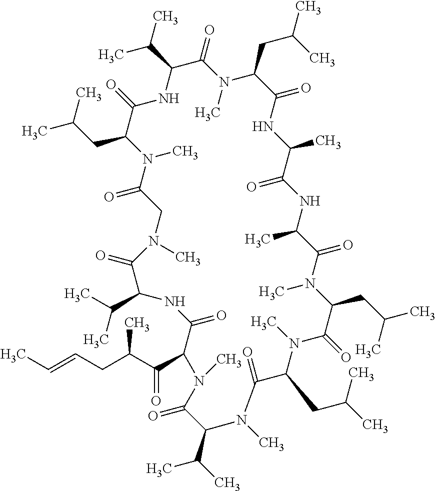 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| BB | Vatalanib succinate | 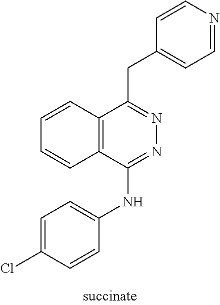 [SMILES: Clc1ccc(Nc2nnc(Cc3ccncc3)c3ccccc23)cc1] | WO 98/35958 |
| CC | | IDH inhibitor | WO2014/141104 |
| DD | | 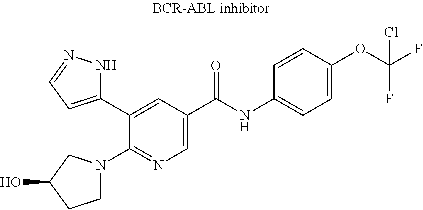 [SMILES: O=C(Nc1ccc(OC(F)(F)Cl)cc1)c1cnc(N2CC[C@@H](O)C2)c(-c2ccn[nH]2)c1] | WO2013/171639 WO2013/171640 WO2013/171641 WO2013/171642 |
| EE | | cRAF inhibitor | WO2014/151616 |
| FF | | ERK1/2 ATP competitive inhibitor | WO2015/066188 |
| | | | PCT/US2014/062913 |
| GG | | 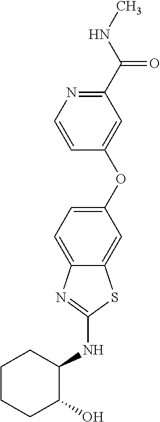 [SMILES: CNC(=O)c1cc(Oc2ccc3nc(N[C@@H]4CCCC[C@H]4O)sc3c2)ccn1] | WO2005/073224 |

### Exemplary Combination Therapies

In certain embodiments, an inhibitor of the immune checkpoint molecule is used in a method or composition described herein. For example, an inhibitor of the immune checkpoint molecule described herein, e.g., the PD-1 inhibitor, e.g., the anti-PD-1 antibody (e.g., Nivolumab or Pembrolizumab); or the PD-L1 inhibitor, e.g., the anti-PD-L1 antibody (e.g., MSB0010718C) (alone or in combination with other immunomodulators) is used in combination with one or more of the agents listed in Table 1; e.g., 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) a transduction modulator and/or apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor. In one embodiment, one or more of the aforesaid combinations is used to treat a disorder, e.g., a disorder described herein (e.g., a disorder disclosed in a publication recited in Table 1). In one embodiment, one or more of the aforesaid combinations is used to treat a cancer, e.g., a cancer described herein (e.g., a cancer disclosed in a publication recited in Table 1).

In some embodiments, one or more of the immunomodulators described herein are used in combination with:
1) 3-(1H-indol-3-yl)-4-[2-(4-methyl-1-piperazinyl)-4-quinazolinyl]-1H-pyrrole-2,5-dione;
2) 5-(2,4-dihydroxy-5-isopropylphenyl)-N-ethyl-4-(4-(morpholinomethyl)phenyl) isoxazole-3-carboxamide;
3) 2-methyl-2-(4-(3-methyl-2-oxo-8-(quinolin-3-yl)-2,3-dihydro-1H-imidazo[4,5-c]quinolin-1-yl)phenyl)propanenitrile
4) Compound D;
5) 4-[3,5-bis(2-hydroxyphenyl)-1H-1,2,4-triazol-1-yl]-benzoic acid;
6) 4,4′-(1H-1,2,4-triazol-1-ylmethylene)bis-benzonitrile;
7) (4S,5R)-3-(2′-amino-2-morpholino-4′-(trifluoromethyl)-[4,5′-bipyrimidin]-6-yl)-4-(hydroxymethyl)-5-methyloxazolidin-2-one;
8) (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one;
9) 4-[(4-methyl-1-piperazinyl)methyl]-N-[4-methyl-3-[[4-(3-pyridinyl)-2-pyrimidinyl]amino]phenyl]-methanesulfonate-benzamide mesylate;
10) 4-[(R)-6,7-dihydro-5H-pyrrolo[1,2-c]imidazol-5-yl]-3-fluorobenzonitrile;
11) N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate;
12) (R)-2-(5-(4-(6-benzyl-4,5-dimethylpyridazin-3-yl)-2-methylpiperazin-1-yl)pyrazin-2-yl)propan-2-ol;
13) Compound M;
14) 2-(2′,3-dimethyl-[2,4′-bipyridin]-5-yl)-N-(5-(pyrazin-2-yl)pyridin-2-yl)acetamide;
15) 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide;
16) Compound P;
17) Compound Q;
18) N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide;
19) 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine;
20) cyclo((4R)-4-(2-Aminoethylcarbamoyloxy)-L-prolyl-L-phenylglycyl-D-tryptophyl-L-lysyl-4-O-benzyl-L-tyrosyl-L-phenylalanyl-);
21) 1-amino-5-fluoro-3-[6-(4-methyl-1-piperazinyl)-1H-benzimidazol-2-yl]-2(1H)-quinolinone;
22) 8-(6-methoxy-pyridin-3-yl)-3-methyl-1-(4-piperazin-1-yl-3-trifluoromethyl-phenyl)-1,3-dihydro-imidazo[4,5-c]quinolin-2-one;
23) N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine;
24) 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide;
25) 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine;
26) 5-chloro-N²-(4-(1-ethylpiperidin-4-yl)-2-fluoro-5-methylphenyl)-N⁴-(5-methyl-1H-pyrazol-3-yl)pyrimidine-2,4-diamine;
27) (3S,6S,9S,12R,15S,18S,21S,24S,30S,33S)-6,9,18,24-tetraisobutyl-3,21,30-triisopropyl-1,4,7,10,12,15,19,25,28-nonamethyl-33-[(2R,4E)-2-methyl-4-hexenoyl]-1,4,7,10,13,16,19,22,25,28,31-undecaazacyclotritriacontane-2,5,8,11,14,17,20,23,26,29,32-undecone;
28) N-(4-chlorophenyl)-4-(4-pyridinylmethyl)-1-phthalazinamine succinate;
29) Compound CC;
30) (R)—N-(4-(chlorodifluoromethoxy)phenyl)-6-(3-hydroxypyrrolidin-1-yl)-5-(1H-pyrazol-5-yl)nicotinamide;
31) Compound EE;
32) Compound FF;
33) 4-((2-(((1R,2R)-2-hydroxycyclohexyl)amino)benzo[d]thiazol-6-yl)oxy)-N-methylpicolinamide.

Each of these combinations is discussed in more detail below.

In one embodiment, the inhibitor of the immune checkpoint molecule (alone or in combination with one or more other immunomodulators) is used in combination with a PKC inhibitor to treat a disorder, e.g., a disorder described herein (e.g., a disorder disclosed in a publication listed in Table 1). In one embodiment, the PKC inhibitor is Sotrastaurin (Compound A) as disclosed herein, or in a publication recited in Table 1. In certain embodiments, the PKC inhibitor is disclosed, e.g., in PCT Publication No. WO 2005/039549, European Patent Application Publication No. EP 1682103, or U.S. Patent Application Publication No. 2007/142401. In one embodiment, Sotrastaurin (Compound A) has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with Sotrastaurin (Compound A) to treat a disorder described herein, e.g., in a publication recited in Table 1, such as a cancer, a melanoma, a non-Hodgkin lymphoma, an inflammatory bowel disease, transplant rejection, an ophthalmic disorder, or psoriasis.

In one embodiment, the PKC inhibitor is a compound of Formula I:

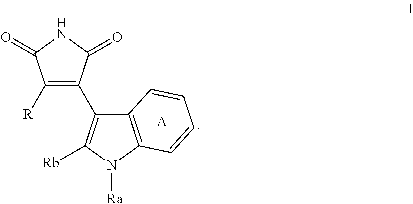

wherein

Rₐ is H; C₁₋₄alkyl; or C₁₋₄alkyl substituted by OH, NH₂, NHC₁₋₄alkyl or N(di-C₁₋₄alkyl)₂;

R_{b} is H; or C₁₋₄alkyl;

R is a radical of formula (a), (b), (c), (d), (e) or (f)

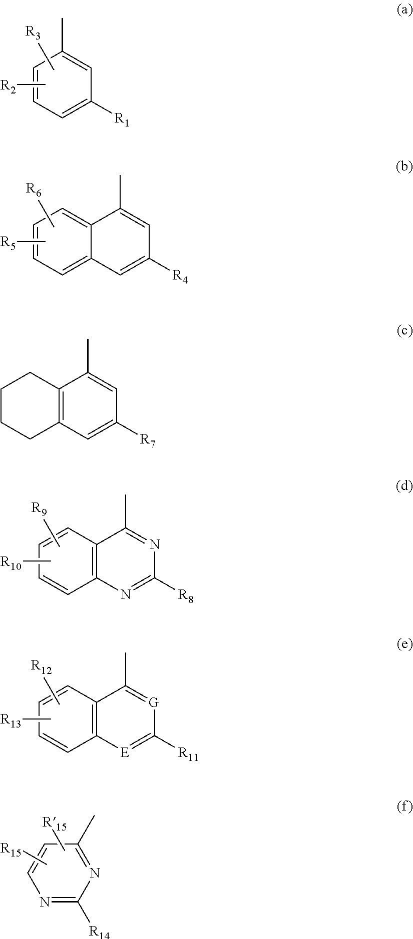

wherein

each of R₁, R₄, R₇, R₈, R₁₁, and R₁₄ is OH, SH, a heterocyclic residue, NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α

—X—R_{c}—Y (α)

wherein X is a direct bond, O, S or NR₁₈ wherein R₁₈ is H or C₁₋₄alkyl,

R_{c} is C₁₋₄alkylene or C₁₋₄alkylene wherein one CH₂ is replaced by CRₓR_{y} wherein one of Rₓ and R_{y} is H and the other is CH₃, each of Rₓ and R_{y} is CH₃ or Rₓ and R_{y} form together CH₂—CH₂—, and

Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₉ and R₂₀ form together with the nitrogen atom to which they are bound a heterocyclic residue;

each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;

either E is —N═ and G is —CH═ or E is —CH═ and G is —N═;

or a salt thereof.

In one embodiment, Sotrastaurin (Compound A) has the following structure:

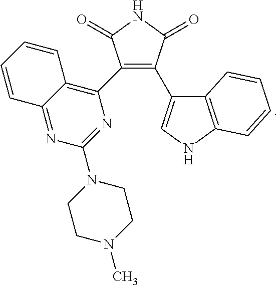 [SMILES: CN1CCN(c2nc(C3=C(c4c[nH]c5ccccc45)C(=O)NC3=O)c3ccccc3n2)CC1]

In one embodiment, Sotrastaurin (Compound A) is 3-(1H-indol-3-yl)-4-[2-(4-methyl-1-piperazinyl)-4-quinazolinyl]-1H-pyrrole-2,5-dione.

In certain embodiments, Sotrastaurin (Compound A) is administered at a dose of about 20 to 600 mg, e.g., about 200 to about 600 mg, about 50 mg to about 450 mg, about 100 mg to 400 mg, about 150 mg to 350 mg, or about 200 mg to 300 mg, e.g., about 50 mg, 100 mg, 150 mg, 200 mg, 300 mg, 400 mg, 500 mg, or 600 mg. The dosing schedule can vary from e.g., every other day to daily, twice or three times a day.

In another embodiment, the inhibitor of an immune checkpoint molecule (alone or in combination with one or more other immunomodulators) is used in combination with an HSP90 inhibitor to treat a disorder, e.g., a disorder described herein (e.g., a disorder disclosed in a publication listed in Table 1). In one embodiment, the HSP90 inhibitor is disclosed herein, e.g., in Table 1. In one embodiment, the HSP90 inhibitor is 5-(2,4-dihydroxy-5-isopropylphenyl)-N-ethyl-4-(4-(morpholinomethyl)phenyl)isoxazole-3-carboxamide (Compound B) as disclosed herein, or in a publication recited in Table 1. In certain embodiments, the HSP90 inhibitor is disclosed, e.g., in PCT Publication No. WO 2010/060937 or WO 2004/072051, European Patent Application Publication No. EP 1611112, or U.S. Pat. No. 8,450,310. In one embodiment, Compound B has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with 5-(2,4-dihydroxy-5-isopropylphenyl)-N-ethyl-4-(4-(morpholinomethyl)phenyl)isoxazole-3-carboxamide (Compound B) to treat a disorder described herein, e.g., in a publication recited in Table 1, such as a cancer, a multiple myeloma, a non-small cell lung cancer, a lymphoma, a gastric cancer, a breast cancer, a digestive/gastrointestinal cancer, a pancreatic cancer, a colorectal cancer, a solid tumor, or a hematopoiesis disorder.

In one embodiment, the HSP90 inhibitor is a compound of formula (A) or (B) or a salt or N-oxide thereof:

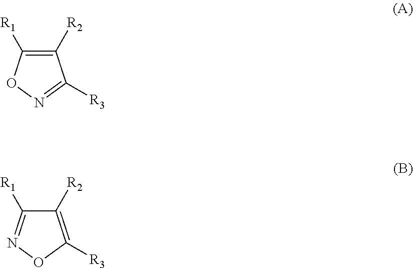

wherein

R₁ is a group of formula (IA)

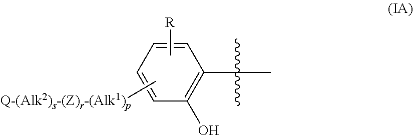

wherein:

R represents one or more optional substituents selected from (C₁-C₆)alkyl, (C₁-C₆)alkoxy, hydroxy, hydroxy(C₁-C₆)alkyl, halo, trifluoromethyl, trifluoromethoxy, oxo, phenyl, —COOH, —COOR^{A}, —COR^{A}—, wherein R^{A} is a (C₁-C₆)alkyl group,

Alk¹ and Alk² are optionally substituted divalent C₁-C₆ alkylene or C₂-C₆ alkenylene radicals,

p, r and s are independently 0 or 1,

Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═)—, —NR^{A}SO₂— or —NR^{A}— wherein R^{A} is hydrogen or C₁-C₆ alkyl, and

Q is hydrogen or an optionally substituted phenyl or pyridinyl radical;

R₂ is (i) a group of formula (IB):

—Ar¹-(Alk¹)ₚ-(Z)ᵣ-(Alk²)ₛ-Q₁ (IB)

wherein:

Ar¹ is an optionally substituted aryl or heteroaryl radical, and

Alk¹, Alk², p, r, s, Z, and R^{A} are as defined in relation to R₁;

Q₁ is hydrogen or an optionally substituted carbocyclic or heterocyclic radical; or

(ii) a carboxamide radical; or

(iii) a non aromatic carbocyclic or heterocyclic ring wherein a ring carbon is optionally substituted, and/or a ring nitrogen is optionally substituted by a group of formula -(Alk¹)ₚ-(Z)ᵣ-(Alk²)ₛ-Q₁, wherein Alk¹, Alk², Z, p, r and s are as defined above in relation to the group of formula (IA) and Q₁ is as defined above in relation to group of formula (IB); and

R₃ is carboxyl, carboxamide, or carboxyl ester group,

wherein the term optionally substituted means substituted with up to four substituents selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo, trifluoromethyl, trifluoromethoxy, nitro, nitrile, oxo, phenyl, —COON, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group.

In one embodiment, Compound B has the following structure:

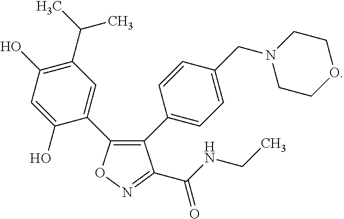 [SMILES: CCNC(=O)c1noc(-c2cc(C(C)C)c(O)cc2O)c1-c1ccc(CN2CCOCC2)cc1]

In one embodiment, Compound B is 5-(2,4-dihydroxy-5-isopropylphenyl)-N-ethyl-4-(4-(morpholinomethyl)phenyl)isoxazole-3-carboxamide.

In another embodiment, the inhibitor of an immune checkpoint molecule (alone or in combination with one or more other immunomodulators) is used in combination with an inhibitor of PI3K and/or mTOR to treat a disorder, e.g., a disorder described herein (e.g., a disorder disclosed in a publication listed in Table 1). In one embodiment, the inhibitor of PI3K and/or mTOR is disclosed herein, e.g., in Table 1, In one embodiment, the inhibitor of PI3K and/or mTOR is Dactolisib (Compound C) or 8-(6-methoxy-pyridin-3-yl)-3-methyl-1-(4-piperazin-1-yl-3-trifluoromethyl-phenyl)-1,3-dihydro-imidazo[4,5-c]quinolin-2-one (Compound V) as described herein, or in a publication recited in Table 1. In certain embodiments, the inhibitor of PI3K and/or mTOR is disclosed, e.g., in PCT Publication No. WO 2006/122806. In one embodiment, Dactolisib (Compound C) or 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound V) has the structure provided in Table 1, or as disclosed in the publication recited in Table 1. In one embodiment, the inhibitor of the immune checkpoint molecule (e.g., one of Nivolumab, Pembrolizumab or MSB0010718C) is used in combination with Dactolisib (Compound C) or 8-(6-Methoxy-pyridin-3-yl)-3-methyl-1-(4-piperazin-1-yl-3-trifluoromethyl-phenyl)-1,3-dihydro-imidazo[4,5-c]quinolin-2-one (Compound V) to treat a disorder described herein, e.g., in a publication recited in Table 1, such as a cancer, a prostate cancer, a leukemia (e.g., lymphocytic leukemia), a breast cancer, a brain cancer, a bladder cancer, a pancreatic cancer, a renal cancer, a solid tumor, or a liver cancer.

In one embodiment, the inhibitor of PI3K and/or mTOR is a compound of formula (I)

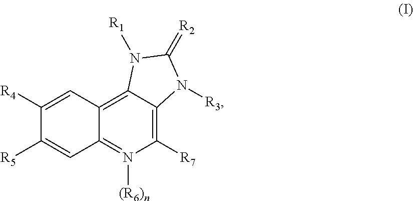

wherein

R₁ is naphthyl or phenyl wherein said phenyl is substituted by one or two substituents independently selected from the group consisting of

halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;

R₂ is O or S;

R₃ is lower alkyl;

R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy;

R₅ is hydrogen or halogen;

n is 0 or 1;

R₆ is oxido;

with the proviso that if n=1, the N-atom bearing the radical R₆ has a positive charge;

R₇ is hydrogen or amino;

or a tautomer thereof, or a pharmaceutically acceptable salt, or a hydrate or solvate thereof.

In one embodiment, Dactolisib (Compound C) has the following structure:

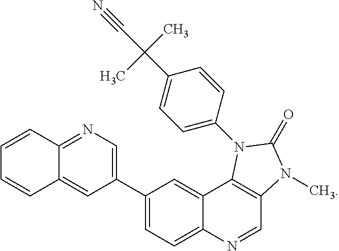 [SMILES: Cn1c(=O)n(-c2ccc(C(C)(C)C#N)cc2)c2c3cc(-c4cnc5ccccc5c4)ccc3ncc21]

In one embodiment, Dactolisib (Compound C) is 2-methyl-2-(4-(3-methyl-2-oxo-8-(quinolin-3-yl)-2,3-dihydro-1H-imidazo[4,5-c]quinolin-1-yl)phenyl)propanenitrile.

In one embodiment, Compound V has the following structure:

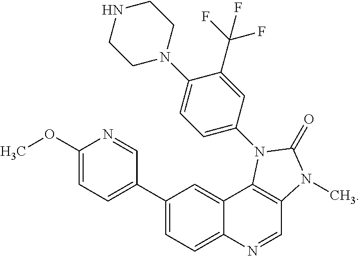 [SMILES: COc1ccc(-c2ccc3ncc4c(c3c2)n(-c2ccc(N3CCNCC3)c(C(F)(F)F)c2)c(=O)n4C)cn1]

In one embodiment, Compound V is 8-(6-methoxy-pyridin-3-yl)-3-methyl-1-(4-piperazin-1-yl-3-trifluoromethyl-phenyl)-1,3-dihydro-imidazo[4,5-c]quinolin-2-one.

In another embodiment, the inhibitor of an immune checkpoint molecule (alone or in combination with one or more other immunomodulators) is used in combination with an inhibitor of cytochrome P450 (e.g., a CYP17 inhibitor) to treat a disorder, e.g., a disorder described herein (e.g., a disorder disclosed in a publication listed in Table 1). In one embodiment, the cytochrome P450 inhibitor (e.g., the CYP17 inhibitor) is disclosed herein, e.g., in Table 1. In one embodiment, the cytochrome P450 inhibitor (e.g., the CYP17 inhibitor) is Compound D as disclosed herein, e.g., a publication recited in Table 1. In certain embodiments, Compound D is disclosed, e.g., in PCT Publication No. WO 2010/149755, U.S. Pat. No. 8,263,635, or European Patent No. 2445903. In one embodiment, the inhibitor of immune check point molecule (e.g., one of Nivolumab, Pembrolizumab or MSB0010718C) is used in combination with Compound D to treat a disorder described herein, e.g., in a publication recited in Table 1 to treat a cancer, e.g., a prostate cancer.

In another embodiment, the inhibitor of an immune checkpoint molecule (alone or in combination with one or more other immunomodulators) is used in combination with an iron chelating agent to treat a disorder, e.g., a disorder described herein (e.g., a disorder disclosed in a publication listed in Table 1). In one embodiment, the iron chelating agent is disclosed herein, e.g., in Table 1. In one embodiment, the iron chelating agent is Deferasirox (Compound E) as disclosed herein, or in a publication recited in Table 1. In certain embodiments, the iron chelating agent is disclosed, e.g., in PCT Publication No. WO 1997/049395. In one embodiment, Defeasirox (Compound E) has the structure provided in Table 1, or as disclosed in the publication recited in Table 1). In one embodiment, the inhibitor of immune checkpoint molecule (e.g., one of Nivolumab, Pembrolizumab or MSB0010718C) is used in combination with Deferasirox (Compound E) to treat a disorder described herein, e.g., in a publication recited in Table 1, e.g., iron overload, hemochromatosis, or myelodysplasia.

In one embodiment, the iron chelating agent is a compound of Formula I:

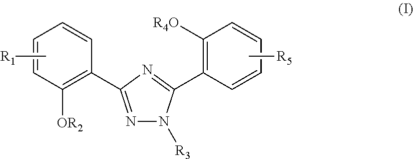

in which

R, and R₅ simultaneously or independently of one another are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;

R₂ and R₄ simultaneously or independently of one another are hydrogen, unsubstituted or substituted lower alkanoyl or aroyl, or a radical which can be removed under physiological conditions;

R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl; R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring; and salts thereof.

In one embodiment, Compound E has the following structure:

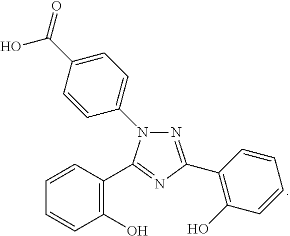 [SMILES: O=C(O)c1ccc(-n2nc(-c3ccccc3O)nc2-c2ccccc2O)cc1]

In one embodiment, Defeasirox (Compound E) is 4-[3,5-bis(2-hydroxyphenyl)-1H-1,2,4-triazol-1-yl]-benzoic acid.

In another embodiment, the inhibitor of an immune checkpoint molecule (alone or in combination with one or more other immunomodulators) is used in combination with an aromatase inhibitor to treat a disorder, e.g., a disorder described herein (e.g., a disorder disclosed in a publication listed in Table 1). In one embodiment, the aromatase inhibitor is disclosed herein, e.g., in Table 1. In one embodiment, the aromatase inhibitor is Letrozole (Compound F) as disclosed herein, or in a publication recited in Table 1. In certain embodiments, the aromatase inhibitor is disclosed, e.g., in U.S. Pat. No. 4,978,672. In one embodiment, Letrozole (Compound F) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with Letrozole (Compound F) to treat a disorder described herein, e.g., in a publication recited in Table 1, such as a cancer, a leiomyosarcoma, an endometrium cancer, a breast cancer, a female reproductive system cancer, or a hormone deficiency.

In one embodiment, the aromatase inhibitor is a compound of formula

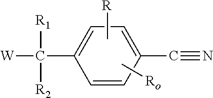

wherein R and Rₒ represent hydrogen or lower alkyl; or R and Rₒ located on adjacent carbon atoms and together when combined with the benzene ring to which they are attached form a naphthalene or tetrahydronaphthalene ring;

R₁ represents hydrogen;

R₂ represents hydrogen, lower alkyl, (lower alkyl, aryl or aryl-lower alkyl)-thio, lower alkenyl, aryl, aryl-lower alkyl, C₃-C₆-cycloalkyl, or C₃-C₆-cycloalkyl-lower alkyl; or R₁ and R₂ combined represent lower alkylidene, mono- or di-aryl-lower alkylidene; R₁ and R₂ combined also represent C₄-C₆-straight chain alkylene, lower alkyl-substituted straight chain alkylene or CH₂-ortho-phenylene-CH₂;

W represents 1-(1,2,4- or 1,3,4))-triazolyl or 1-(1,2,4 or 1,3,4-triazolyl substituted by lower alkyl; aryl within the above definitions represents phenyl or phenyl substituted by one or two substituents selected from lower alkyl, lower alkoxy, hydroxy, lower alkanoyloxy, aroyloxy, nitro, amino, halogen, trifluoromethyl, cyano, carboxy, carboxy functionalized in form of a pharmaceutically acceptable ester or amide, lower alkanoyl, aroyl, lower alkylsulfonyl, sulfamoyl, N-lower alkylsulfamoyl or N,N-di-lower alkylsulfamoyl; and aryl within the above definitions also represents 2-, 3-, or 4-pyridyl or a said heterocyclic radical monosubstituted by lower alkyl, lower alkoxy, cyano or halogen; and aroyl within the above definitions represents benzoyl or benzoyl substituted by lower alkyl, lower alkoxy, halogen or trifluoromethyl;

or a pharmaceutically acceptable salt thereof.

In one embodiment, Letrozole (Compound F) has the following structure:

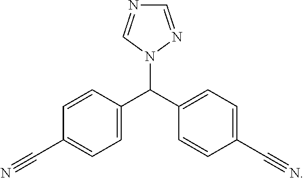 [SMILES: N#Cc1ccc(C(c2ccc(C#N)cc2)n2cncn2)cc1]

In one embodiment, Letrozole (Compound F) is 4,4′-(1H-1,2,4-triazol-1-ylmethylene)bis-benzonitrile.

In another embodiment, the inhibitor of an immune checkpoint molecule (alone or in combination with one or more other immunomodulators) is used in combination with a PI3K inhibitor, e.g., a pan-PI3K inhibitor, to treat a disorder, e.g., a disorder described herein (e.g., a disorder disclosed in a publication listed in Table 1). In one embodiment, the PI3K inhibitor is disclosed herein, e.g., in Table 1. In one embodiment, the PI3K inhibitor is (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) as disclosed herein, e.g., in a publication recited in Table 1. In certain embodiments, the PI3K inhibitor is disclosed, e.g., in PCT Publication No. WO 2013/124826 or U.S. Patent Application Publication No. 2013/0225574. In one embodiment, (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) has the structure provided in Table 1, or as disclosed in a publication recited in Table 1. In one embodiment, the immune checkpoint molecule (e.g., one of Nivolumab, Pembrolizumab or MSB0010718C) is used in combination with (4S,5R)-3-(2′-amino-2-morpholino-4′-(trifluoromethyl)-[4,5′-bipyrimidin]-6-yl)-4-(hydroxymethyl)-5-methyloxazolidin-2-one (Compound G) to treat a disorder described herein, e.g., in a publication recited in Table 1, such as a cancer or an advanced solid tumor.

In one embodiment, the PI3K inhibitor is a compound of Formula (I)

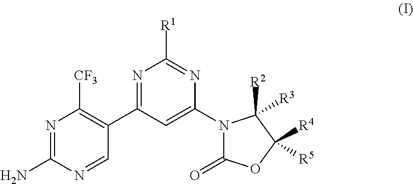

wherein,

R¹=

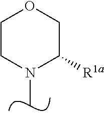

wherein R^{1a}=H or —CH

or R¹=

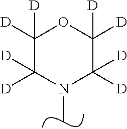

wherein D=deuterium;

R²=H and R³=H;

R⁴=H, and R⁵=—CH₃ or —CH₂OH; or

R⁴=—CH₂OH, and R⁵=H; or

R²=—CH₃, —CH₂OH, —CH₂OCH₃, —CH₂CH₂OH or —CH₂OC(O)H;

R³=H;

R⁴=—CH₃, —CH₂OH, —CH₂CH₂OH, —CH₂CH(OH)CH₃ or —CH₂C(OHXCH₃)₂ and R⁵=H, or R⁴=H, and R⁵=—CH₃, —CH₂CH—CH₂CH(OH)CH₃ or —CH₂C(OH)(CH₃)₂ₗ or

R⁴=H or —CH3 and R⁵=H or —CH₃; or

R³=H and R⁴=H;

R² and R⁵ are joined and form —(CH₂)4-; or

R⁴=H and R⁵=H; and

R²=—CH₂OH, and R³=—CH₃; or

R²=H or —CH₃, and R³=—CH₂OH; or R=H and R⁴=H; and

R³ and R⁵ are joined and form the group

or the group

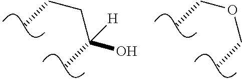

or

R³=H and R⁵=H; and

R² and R⁴ are joined and form the group

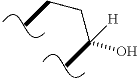

a pharmaceutically acceptable salt thereof.

In one embodiment, Compound G has the following structure:

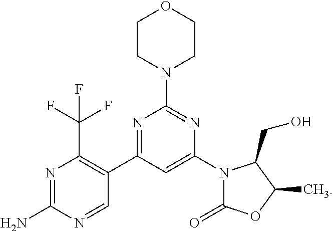 [SMILES: C[C@H]1OC(=O)N(c2cc(-c3cnc(N)nc3C(F)(F)F)nc(N3CCOCC3)n2)[C@H]1CO]

In one embodiment, Compound G is (4S,5R)-3-(2′-amino-2-morpholino-4′-(trifluoromethyl)-[4,5′-bipyrimidin]-6-yl)-4-(hydroxymethyl)-5-methyloxazolidin-2-one.

In another embodiment, the inhibitor of an immune checkpoint molecule (alone or in combination with one or more other immunomodulators) is used in combination with an inhibitor of p53, e.g., an inhibitor of a p53/Mdm2 interaction, to treat a disorder, e.g., a disorder described herein (e.g., a disorder disclosed in a publication listed in Table 1). In one embodiment, the inhibitor of p53, e.g., an inhibitor of a p53/Mdm2 interaction, is disclosed herein, e.g., in Table 1. In one embodiment, the inhibitor of p53, e.g., an inhibitor of a p53/Mdm2 interaction, is (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) as disclosed herein, or in a publication recited in Table 1. In certain embodiments, the inhibitor of p53, e.g., an inhibitor of a p53/Mdm2 interaction, is disclosed, e.g., in PCT Publication No. WO2013/111105. In one embodiment, (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (Compound H) to treat a disorder described herein, e.g., in publication reicted in Table 1, such as a cancer or a soft tissue sarcoma.

In one embodiment, the inhibitor of p53, e.g., an inhibitor of a p53/Mdm2 interaction, is a compound of formula (I) or a salt thereof,

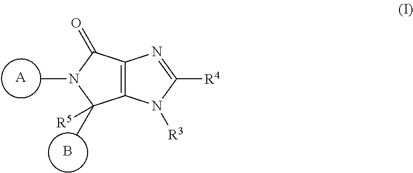

wherein

A is selected from:

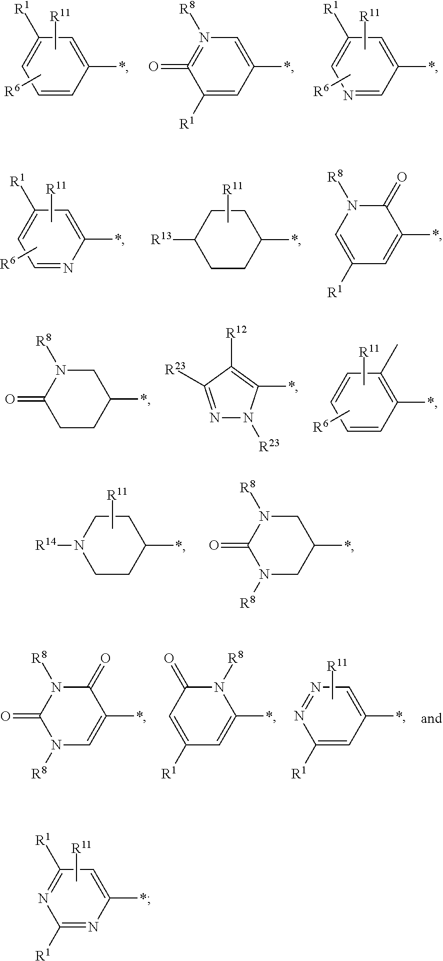

B is selected from:

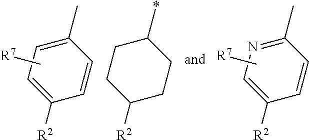

each R is independently selected from halo and methyl;

each R¹ and R² is independently selected from chloro, fluoro, trifluoromethyl, methyl and cyano;

R³ is selected from isopropyl, cyclopropyl, isobutyl, cyclobutyl and cyclopentyl, or R³ is:

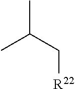

wherein R²² is selected from OH, OCH₃, NH₂, NHMe, NMe₂, NHCOMe and NHCOH;

R⁴ is elected from:

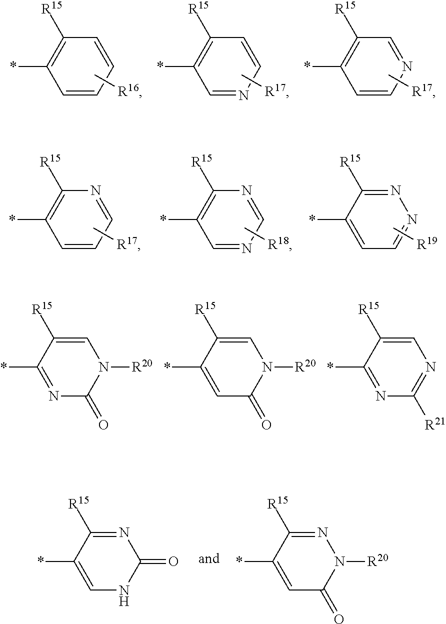

wherein

R¹⁵ is independently selected from OCH₃, CH₂CH₃, OH, OCF₃ and H;

R¹⁶ is selected from H, O—(C₁-C₄)alkyl, halo, OCF₃, CN, —C(O)NR⁹R¹⁰, —C(O)— morpholinyl-4-yl, hydroxy-azetidin-1-yl-carbonyl, —CH₂NR⁹R¹⁰, —CH₂NR⁹—C(O)R¹⁰, CH₂CN, methyl-imidazolyl-, —CH₂C(O)NR⁹R¹⁰, —CH₂C(O)OH, —C(O)OH, —CH₂C(O)O—(CC₄)alkyl, —N(R⁹)—C(O)—(C₁C₄)alkyl, —NR⁹R¹⁰ and (C₁-C₄)alkyl optionally substituted by 1 or 2 OH;

R¹⁷ is selected from H, O(C₁-C₄)alkyl, —CH₂C(O)NR⁹R¹⁰, —CH₂C(O)O—(C₁-C₄)alkyl, —CH₂C(O)OH, NR⁹R¹⁰, —C(O)NR⁹R¹⁰, —CH₂NR⁹R¹⁰, —C(O)OCH₃ and —CH₂CN;

R¹⁸ is selected from H, O(C₁-C₄)alkyl, OH, CH₂NR⁹R¹⁰, —NR⁹R¹⁰ and azetidin-1-yl, said azetidin- being substituted with OH or both CH₃ and OH,

R¹⁹ is selected from H, O(C₁-C₄)alkyl, (C₁-C₄)alkyl, —NR⁹R¹⁰, —N(R⁹)—C(O)—(C₁-C₄)alkyl and —C(O)NR⁹R¹⁰;

R²⁰ is selected from H, CH₃ and —CH₂CH₃;

R²¹ is selected from —NR⁹R¹⁰, —CH₂NR⁹R¹⁰, C(O)NR⁹R¹⁰ and CN;

R⁵ is selected from H, heterocyclyl —C(O)—(CH₂)ₙ—, (C₁-C₄)alkyl-, said (C₁-C₄)alkyl- being optionally substituted with 1 or 2 substituents independently selected from OH, O; heterocyclyl ¹-(C₁-C₄)alkyl-, wherein said alkyl of heterocyclyl¹ —(C₁-C₄)alkyl- is optionally substituted by 1 or 2 OH, and said heterocyclyl¹ can be optionally substituted by methyl or ethyl; (C₁-C₄)alkyl-O—C(O)—(CH₂)ₘ—, and cyano;

R⁶ is selected from H, (C₁-C₄)alkyl-, optionally substituted with (C₁-C₄)alkoxy; (C₁-C₄)alkoxy, optionally substituted with (C₁-C₄)alkoxy, (C₁-C₄)alkoxy(C₁-C₄)alkoxy(C₁-C₄)alkyl-; halo; R⁹(R¹⁰)N—C(O)—(CH₂)ₘ—; cyano; R⁹(R¹⁰)N—(CH₂)ₘ—; R⁹(R¹⁰)N—(CH₂)ₙ—O—(CH₂)ₘ—; (C₁-C₄)alkyl-C(O)—(R¹⁰)N—(CH₂)ₘ—; O—(CH₂)ₚ-heteroaryl²;

R⁷ is selected from H; halo; and (C₁-C₄)alkyl-, optionally substituted with (C₁-C₄)alkoxy;

each R⁸ is independently selected from H, methyl, ethyl, hydroxyethyl and methoxyethyl, wherein said methyl or ethyl is optionally substituted with 1, 2 or 3 fluoro substituents;

each R⁹ is independently selected from H, methyl or ethyl;

each R¹⁰ is independently selected from H and (C₁-C₄) alkyl wherein said (C₁-C₄) alkyl is optionally substituted by 1 or 2 substituents independently selected from methoxy, ethoxy, hydroxy and halo; or R⁹ and R¹⁰, together with the N atom to which they are attached, can join to form a saturated 5 or 6 membered heterocyclic ring further comprising ring carbon atoms and optionally one ring heteroatom independently selected from N, O and S, and wherein when the ring contains a S atom, said S is optionally substituted with one or two oxo substituents;

R¹¹ is H, (C₁C₄)alkyl, (C₁-C₄) alkoxy or halo; R² is H or halo;

R¹³ is selected from NH₂, —C(O)OH, —NH(C(O)—CH₃) and —C(O)—NH(CH₃);

R¹⁴ is selected from —C(O)—NR⁹(R¹⁰), (C₁-C₄)alkyl, —C(O)(C₁-C₄)alkyl, —C(O)O(C₁-C₄)alkyl;

each R²³ is independently selected from H, halo, cyclopropyl and (C₁-C₄)alkyl; n is 1, 2 or 3;

p is 0, 1, 2 or 3;

heterocyclyl¹ is a 3, 4, 5 or 6 membered fully saturated or partially unsaturated monocyclic group comprising ring carbon atoms and 1 or 2 ring heteroatoms independently selected from N, O and S;

heteroaryl² is 5 or 6 membered fully unsaturated monocyclic group comprising ring carbon atoms and 1, 2, 3 or 4 ring heteroatoms independently selected from N, O and S, wherein the total number of ring S atoms does not exceed 1, and the total number of ring O atoms does not exceed 1; and m is 0, 1 or 2.

* indicates the point of attachment to the remainder of the molecule.

In an embodiment, Compound H has the following structure:

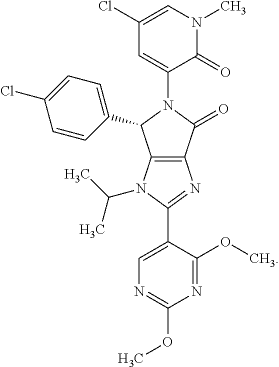 [SMILES: COc1ncc(-c2nc3c(n2C(C)C)[C@H](c2ccc(Cl)cc2)N(c2cc(Cl)cn(C)c2=O)C3=O)c(OC)n1]

In an embodiment, Compound H is (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one.

In another embodiment, the inhibitor of an immune checkpoint molecule (alone or in combination with one or more other immunomodulators) is used in combination with an apoptosis inducer and/or an angiogenesis inhibitor to treat a disorder, e.g., a disorder described (e.g., a disorder disclosed in a publication listed in Table 1). In one embodiment, the apoptosis inducer and/or an angiogenesis inhibitor is disclosed herein, e.g., in Table 1. In one embodiment, the apoptosis inducer and/or angiogenesis inhibitor is Imatinib mesylate (Compound I) as disclosed herein, or in a publication recited in Table 1. In certain embodiments, the apoptosis inducer and/or angiogenesis inhibitor is disclosed, e.g., in PCT Publication No. WO1999/003854. In one embodiment, the apoptosis inducer and/or an angiogenesis inhibitor has the structure provided in Table 1, or as disclosed in a publication disclosed in Table 1. In one embodiment, the inhibitor of immune checkpoint molecule (e.g., one of Nivolumab, Pembrolizumab or MSB0010718C) is used in combination with Imatinib mesylate (Compound I) to treat a disorder described herein, e.g., in a publication recited in Table 1, such as a cancer, a multiple myeloma, a prostate cancer, a non-small cell lung cancer, a lymphoma, a gastric cancer, a melanoma, a breast cancer, a pancreatic cancer, a digestive/gastrointestinal cancer, a colorectal cancer, a glioblastoma multiforme, a liver cancer, a head and neck cancer, asthma, multiple sclerosis, allergy, Alzheimer's dementia, amyotrophic lateral sclerosis, or rheumatoid arthritis.

In one embodiment, Imatinib mesylate (Compound I) has the following structure:

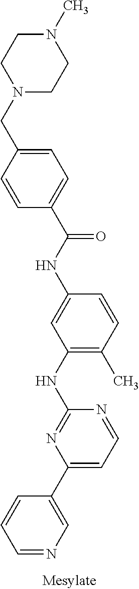 [SMILES: Cc1ccc(NC(=O)c2ccc(CN3CCN(C)CC3)cc2)cc1Nc1nccc(-c2cccnc2)n1]

In one embodiment, Imatinib mesylate (Compound I) is 4-[(4-methyl-1-piperazinyl)methyl]-N-[4-methyl-3-[[4-(3-pyridinyl)-2-pyrimidinyl]amino]phenyl]-methanesulfonate-benzamide mesylate.

In certain embodiments, Imatinib mesylate (Compound I) is administered at a dose of about 100 to 1000 mg, e.g., about 200 mg to 800 mg, about 300 mg to 700 mg, or about 400 mg to 600 mg, e.g., about 200 mg, 300 mg, 400 mg, 500 mg, 600 mg, or 700 mg. The dosing schedule can vary from e.g., every other day to daily, twice or three times a day. In one embodiment, Imatinib mesylate is administered at an oral dose from about 100 mg to 600 mg daily, e.g., about 100 mg, 200 mg, 260 mg, 300 mg, 400 mg, or 600 mg daily.

In another embodiment, the inhibitor of an immune checkpoint molecule (alone or in combination with one or more other immunomodulators) is used in combination with an inhibitor of one or more of cytochrome P450 (e.g., 11B2), aldosterone or angiogenesis to treat a disorder, e.g., a disorder described herein (e.g., in a disorder disclosed in a publication listed in Table 1). In one embodiment, the inhibitor of one or more of cytochrome P450 (e.g., 11B2), aldosterone or angiogenesis is disclosed herein, e.g., in Table 1. In one embodiment, the inhibitor of one or more of cytochrome P450 (e.g., 11B2), aldosterone or angiogenesis is Osilodrostat (Compound J) as disclosed herein, or in a publication recited in Table 1. In certain embodiments, the inhibitor of one or more of cytochrome P450 (e.g., 11B2), aldosterone or angiogenesis is disclosed, e.g., in PCT Publication No. WO2007/024945. In one embodiment, Osilodrostat (Compound J) has the structure provided in Table 1, or as disclosed in a publication recited in Table 1. In one embodiment, the immune checkpoint molecule (e.g., one of Nivolumab, Pembrolizumab or MSB0010718C) is used in combination with Osilodrostat (Compound J) to treat a disorder described herein, e.g., in a publication recited in Table 1, such as Cushing's syndrome, hypertension, or heart failure therapy.

In one embodiment, the inhibitor of one or more of cytochrome P450 (e.g., 11B2), aldosterone or angiogenesis is a compound of formula (I)

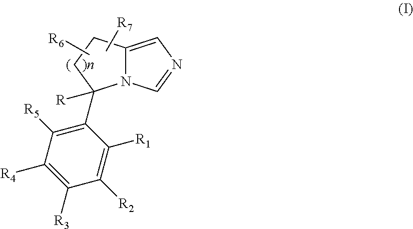

wherein

n is 1, or 2, or 3;

R is hydrogen, (C₁-C₇) alkyl, or (C₁-C₇) alkenyl, said (C₁-C₇) alkyl and (C₁-C₇) alkenyl being optionally substituted by one to five substituents independently selected from the group consisting of —O—R₈ and —N(R₈)(R₉), wherein R₈ and R₉ are independently selected from the group consisting of hydrogen, (C₁-C₇) alkyl, acyl, aryl and heteroaryl, each of which is further optionally substituted by one to four substituents independently selected from the group consisting of halo, (C₁-C₇) alkoxy and (C₁-C₇) alkyl; or

R is —C(O)O—R₁₀, or —C(O)N(Rii)(Ri₂), wherein R₁₀, Rn and R₁₂ are selected independently from the group consisting of hydrogen, (C₁-C₇) alkyl, (C₃-C₈) cycloalkyl, aryl, aryl-(CrC₇) alkyl, (C₁-C₇) haloalkyl and heteroaryl, each of which is further optionally substituted by one to four substituents independently selected from the group consisting of halo, hydroxyl, (C₁-C₇) alkoxy, (C₁-C₇) alkyl, and aryl, wherein R₁—, and R₁₂ taken together with the nitrogen atom to which they are attached optionally form a 3-8-membered ring;

R₁, R₂, R₃, R₄, and R₅ are selected independently from the group consisting of hydrogen, (C₁-C₇) alkenyl, (C₁-C₇) alkyl, (C₃-C₈) cycloalkyl, halo, cyano, nitro, H₂N—, (C₁-C₇) haloalkyl, (C₁-C₇) alkoxy, (C₃-C₈) cycloalkoxy, aryloxy, aryl, heretoaryl, —C(O)OR₁₀, and —N(R₁₃)(Ri₄), said (C₁-C₇) alkyl, (C₁-C₇) alkenyl, (C₁-C₇) alkoxy, aryl and heteroaryl being further optionally substituted by one to three substituents selected from (C₁-C₇) alkyl, hydroxyl, halo, (C₁-C₇) alkoxy, nitro, cyano, (Ci-C₇) dialkylamino, (C₁-C₇) alkoxy- (C₁-C₇) alky-, and (C₁-C₇) haloalkyl, said Ri₀ having the same meanings as defined above, said R₁₃ and R₁₄ are independently selected from the group consisting of hydrogen, (C₁-C₇) alkyl, (C₃-C₈) cycloalkyl, (C₁-C₇) haloalkyl, (C₁-C₇) haloalkoxy, aryl and cyano, with the proviso that no more than three of Ri₁ R₂, R₃, R₄, and R₅ are simultaneously hydrogen;

R₁₃ and Rᵢ₄ taken together with the nitrogen atom to which they are attached optionally form a 3-8-membered ring;

R and R₁ taken together optionally form a 5-6-membered ring containing 0 or 1 heteroatom selected from O, N, or S;

R₆ and R₇ are independently hydrogen, hydroxyl, (C₁-C₇) alkyl, (C₁-C₇) alkoxy, phenyl, or benzyl, wherein phenyl and benzyl are optionally substituted by one to four substituents independently selected from the group consisting of halo, (C₁-C₇) alkoxy and (C₁-C₇) alkyl;

when R₆ and R₇ are attached to the same carbon atom, they optionally form a moiety (A) represented by the following structure:

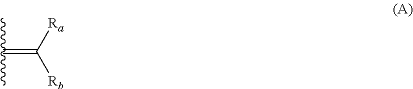

wherein Rₐ and R_{b} are independently hydrogen, (C₁-C₇) alkyl, (C₁-C₇) alkoxy, acyl, —COOR₁₅ or —COR₁₅, said R₁₅ being hydrogen, (C₁-C₇) alkyl, (C₁-C₇) haloalkyl, aryl, or —NH₂; or

when R₆ and R₇ are attached to the same carbon atom, they taken together with said carbon atom optionally form a 3-8-membered ring; or

a pharmaceutically acceptable salt thereof; or an optical isomer thereof; or a mixture of optical isomers. a pharmaceutically acceptable salt thereof; or an optical isomer thereof; or a mixture of optical isomers.

In one embodiment, Osilodrostat (Compound J) has the following structure:

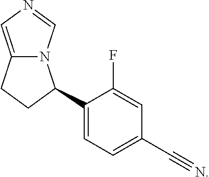 [SMILES: N#Cc1ccc([C@H]2CCc3cncn32)c(F)c1]

In one embodiment, Osilodrostat (Compound J) is 4-[(R)-6,7-dihydro-5H-pyrrolo[1,2-c]imidazol-5-yl]-3-fluorobenzonitrile.

In another embodiment, the inhibitor of an immune checkpoint molecule (alone or in combination with one or more other immunomodulators) is used in combination a Smoothened (SMO) inhibitor to treat a disorder, e.g., a disorder described herein (e.g., a disorder disclosed in a publication listed in Table 1). In one embodiment, the SMO inhibitor is disclosed herein, e.g., in Table 1. In one embodiment, the SMO inhibitor is Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) as disclosed herein, or in a publication recited in Table 1. In certain embodiments, the SMO inhibitor is disclosed, e.g., in PCT Publication No. WO 2007/131201 or WO 2010/007120, European Patent Application Publication No. EP 2021328, or U.S. Pat. No. 8,178,563. In certain embodiments, Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) has the structure provided in Table 1, or as disclosed in a publication recited in Table 1. In one embodiment, the inhibitor of immune checkpoint molecule (e.g., one of Nivolumab, Pembrolizumab or MSB0010718C) is used in combination with Sonidegib phosphate (Compound K) or (R)-2-(5-(4-(6-benzyl-4,5-dimethylpyridazin-3-yl)-2-methylpiperazin-1-yl)pyrazin-2-yl)propan-2-ol (Compound L) to treat a disorder described herein, in a publication recited in Table 1, such as a cancer, a medulloblastoma, a small cell lung cancer, a prostate cancer, a basal cell carcinoma, a pancreatic cancer, or an inflammation.

In one embodiment, the SMO inhibitor is a compound of Formula I:

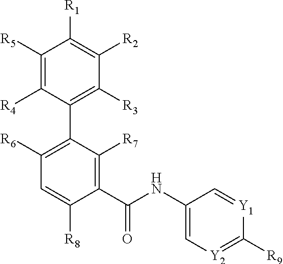

in which:

Y₁ and Y₂ are independently selected from N and CR₁₀; wherein R₁₀ is selected from hydrogen, halo, C₁-C₆alkyl, halosubstituted-C₁C₆alkyl, C₁C₆alkoxy, halosubstituted-C₁₋₆alkoxy and OXNR₁₀ₐR_{10b}; wherein R₁₀ₐ and R_{10b} are independently selected from hydrogen and C₁C₆alkyl;

R₁ is selected from cyano, halo, C₁C₆alkyl, halosubstituted-C₁C₆alkyl, C₁-C₆alkoxy, halosubstituted-C₁C₆alkoxy, C₆₋₁₀aryl, dimethyl-amino, C₁₋₆alkyl-sulfanyl and C₃₋₈heterocycloalkyl optionally substituted with up to 2 C₁₋₆alkyl radicals;

R₂ and R₅ are independently selected from hydrogen, cyano, halo, C₁₋₆alkyl, halosubstituted-C₁₋₆ alkyl, Ci_{—6}alkoxy, halosubstituted-C₁₋₆alkoxy and dimethylamino;

R₃ and R₄ are independently selected from hydrogen, halo, cyano, C₁₋ βalkyl, halosubstituted-Ci_{—6}alkyl, Ci-₆alkoxy and halosubstituted-C₁₋₆alkoxy; or either R₁ and R₂ or R₁ and R₅ together with the phenyl to which they are both attached form C₅₋₁₀heteroaryl;

R₆ and R₇ are independently selected from hydrogen, C₁₋₆alkyl, halosubstituted-C₁₋₆alkyl, C₁₋₆alkoxy and halosubstituted-C₁₋₆-alkoxy; with the proviso that R₆ and R₇ are not both hydrogen;

R₈ is selected from halo, C₁₋₆alkyl, halosubstituted-C₁₋₆alkyl, C₁₋₆alkoxy and halosubstituted-C₁₋₆alkoxy;

R₉ is selected from —S(O)₂Rn, —C(O)Rₙ, —NR₁₂ₐR_{12b} and —R₁₁; wherein R₁₁ is selected from aryl, heteroaryl, cycloalkyl and heterocycloalkyl; R₁₂ₐ and R_{12b} are independently selected from C₁₋₆alkyl and hydroxy-substituted-C₁₋₆alkyl;

wherein said aryl, heteroaryl, cycloalkyl and heterocycloalkyl of R₉ can be optionally substituted with 1 to 3 radicals independently selected from C₁₋₆alkyl, halosubstituted-C₁₋₆alkyl, C₁₋₆alkoxy, halosubstituted-C₁₋₆alkoxy, C₆-ioaryl-C₀₋₄alkyl, C₅₋₁₀heteroaryl-C₀₋₄alkyl, C₃₋₁₂cycloalkyl and C₃₋₈heterocycloalkyl; wherein said aryl-alkyl substituent of R₉ is optionally substituted with 1 to 3 radicals independently selected from halo, Ci_{—6}alkyl, halosubstituted-C₁₋₆ alkyl, C₁₋₆alkoxy, halosubstituted-C₁₋₆alkoxy and methyl-piperazinyl; and the pharmaceutically acceptable salts, hydrates, solvates and isomers thereof.

In one embodiment, Sonidegib phosphate (Compound K) has the following structure:

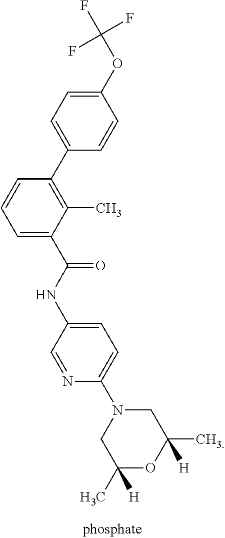 [SMILES: Cc1c(C(=O)Nc2ccc(N3C[C@@H](C)O[C@@H](C)C3)nc2)cccc1-c1ccc(OC(F)(F)F)cc1]

In one embodiment, Sonidegib phosphate (Compound K) is N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate.

In one embodiment, the SMO inhibitor is compound of Formula I:

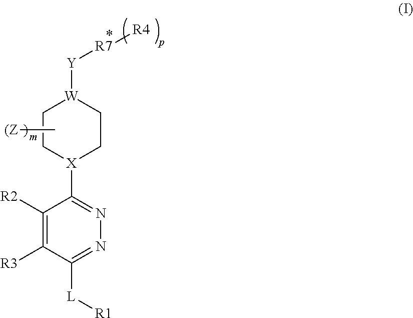

or a pharmaceutically acceptable salt thereof, wherein

R1 is a C₆₋₁₄ aryl group, or a 5-14 membered heteroaryl group, each of which may be

unsubstituted or substituted by one or more of C₁₋₈ alkyl, a C₆₋₁₄ aryl group, C₁₋₈ haloalkyl, C₁₋₈ alkoxy, halo, NH2, CN, OCF3, OH, C(O)NR6R8, C(O)R6, NR6R8, NHC(O)R6, SO₂R6, SO₂NR6R8;

R2 and R3 are independently C₁₋₈ alkyl, C₁₋₈alkylOH, or R2 and R3 form C₃₋₁₄ cycloalkyl group;

L is a bond, C₁₋₈ alkylene, —C(O)O—, —CONR9-, —C₁₋₈ alkylOH—, C₁₋₈ haloalkyl, —C(O)—, —NH— or —O—;

X and W are independently N, or CR5 and at least one of X and W is N;

R7 is a C₆₋₁₄ aryl group, a 5-14 membered heteroaryl group, or a 3-14 membered cycloheteroalkyl group;

R4 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo, NR6R8, C(O)OR6, C(O)NR6R8, C₁₋₈ haloalkyl, formyl, carbalkoxy, C₁₋₈ alkylOH, C(O)R6, SO₂R6, C(O)NHC₁₋₈alkylR6, NR6R8, SO₂NR6R8, OCF3, NHC(O)R6, CH₂OC(O)NR6R8, CH2NR6R8, NHC(O)OR6, NHC(O)NR6R8, CH₂NHSO₂R6, CH₂NHC(O)OR6, OC(O)R6, or NHC(O)R6, which may be substituted or unsubstituted;

Z is C₁₋₈ alkyl, CN, OH, or halogen;

m and p are independently 0-3;

Y is a bond, C₁₋ₛ alkylene, —C(O)—, —C(O)O—, —CH(OH)—, or —C(O)N(R10)-;

R5 is H, halogen, CN, lower alkyl, OH, OCH3 or OCF3;

R9 and R10 are independently C₁₋₈ alkyl or H;

R6 and R8 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ haloalkyl, C₁₋₈alkylOH, C₁₋₈alkoxy, or R6 and R8 on one atom can form a heteroatom containing ring; and

wherein R4, R6, and R8 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH3, —OCF3, —OH, —NH2.

In one embodiment, Compound L has the following structure:

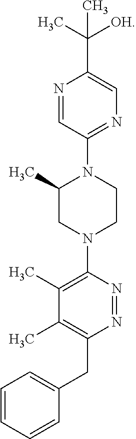 [SMILES: Cc1c(Cc2ccccc2)nnc(N2CCN(c3cnc(C(C)(C)O)cn3)[C@H](C)C2)c1C]

In one embodiment, Compound L is (R)-2-(5-(4-(6-benzyl-4,5-dimethylpyridazin-3-yl)-2-methylpiperazin-1-yl)pyrazin-2-yl)propan-2-ol.

In certain embodiments, Sonidegib phosphate (Compound K) is administered at a dose of about 20 to 500 mg, e.g., about 40 mg to 400 mg, about 50 mg to 300 mg, or about 100 mg to 200 mg, e.g., about 50 mg, 100 mg, 150 mg, 200 mg, 250 mg, or 300 mg. The dosing schedule can vary from e.g., every other day to daily, twice or three times a day.

In another embodiment, the inhibitor of an immune checkpoint molecule (alone or in combination with one or more other immunomodulators) is used in combination a prolactin receptor (PRLR) inhibitor to treat a disorder, e.g., a disorder described herein (e.g., a disorder disclosed in a publication listed in Table 1). In one embodiment, the PRLR inhibitor is disclosed herein, e.g., in Table 1. In one embodiment, the PRLR inhibitor is a human monoclonal antibody (Compound M) disclosed herein, e.g., or in a publication recited in Table 1. In certain embodiments, the human monoclonal antibody (Compound M) is disclosed, e.g., in U.S. Pat. No. 7,867,493. In one embodiment, the inhibitor of the immune checkpoint molecule (e.g., one of Nivolumab, Pembrolizumab or MSB0010718C) is used in combination with human monoclonal antibody molecule (Compound M) described in U.S. Pat. No. 7,867,493 to treat a disorder described herein, in a publication recited in Table 1, such as, a cancer, a prostate cancer, or a breast cancer.

In one embodiment, the PRLR inhibitor is an anti-PRLR antibody molecule.

In one embodiment, Compound M is an isolated antibody that binds the extracellular domain of PRLR of SEQ ID NO: 2 of U.S. Pat. No. 7,867,493 with an equilibrium dissociation constant (K_{D}) of 10⁻⁶ M or lower and that comprises (a) the Complementarily Determining Regions (CDRs) set forth at positions 24 through 38, positions 54 through 60, and positions 93 through 101 of the amino acid sequence of SEQ ID NO: 88 of U.S. Pat. No. 7,867,493 and (b) the CDRs set forth at positions 31 through 35, positions 50 through 66, and 99 through 113 of SEQ ID NO: 90 of U.S. Pat. No. 7,867,493.

In one embodiment, Compound M is an isolated antibody that binds the extracellular domain of PRLR comprising a variable light chain amino acid sequence SEQ ID NO: 88 of U.S. Pat. No. 7,867,493, and a variable heavy chain amino acid sequence of SEQ ID NO: 90 of U.S. Pat. No. 7,867,493.

In another embodiment, the inhibitor of an immune checkpoint molecule (alone or in combination with one or more other immunomodulators) is used in combination a Wnt signaling inhibitor to treat a disorder, e.g., a disorder described herein (e.g., a disorder disclosed in a publication listed in Table 1). In one embodiment, the Wnt signaling inhibitor is disclosed herein, e.g., in Table 1. In one embodiment, the Wnt signaling inhibitor is 2-(2′,3-dimethyl-[2,4′-bipyridin]-5-yl)-N-(5-(pyrazin-2-yl)pyridin-2-yl)acetamide (Compound N) as disclosed herein, or in a publication recited in Table 1. In certain embodiments, 2-(2′,3-dimethyl-[2,4′-bipyridin]-5-yl)-N-(5-(pyrazin-2-yl)pyridin-2-yl)acetamide (Compound N) is disclosed, e.g., in PCT Publication No. WO 2010/101849. In one embodiment, the inhibitor of the immune checkpoint molecule (e.g., one of Nivolumab, Pembrolizumab or MSB0010718C) is used in combination with 2-(2′,3-dimethyl-[2,4′-bipyridin]-5-yl)-N-(5-(pyrazin-2-yl)pyridin-2-yl)acetamide (Compound N) to treat a disorder described herein, in a publication disclosed in Table 1, such as a cancer or a solid tumor (e.g., a head and neck cancer, a squamous cell carcinoma, a breast cancer, a pancreatic cancer, or a colon cancer).

In one embodiment, the Wnt signaling inhibitor is a compound having Formula (1) or (2):

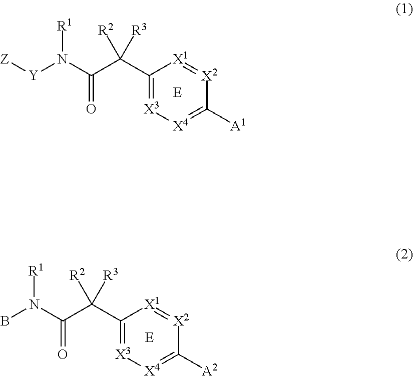

or a physiologically acceptable salt thereof, wherein: ring E is an optionally substituted aryl or heteroaryl;

A¹ and A² are independently a heterocycle, quinolinyl, or a heteroaryl selected from the group

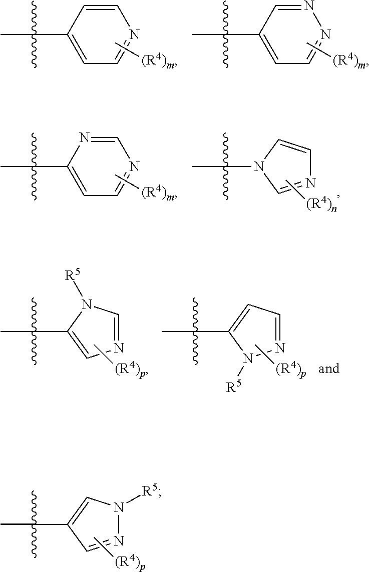

wherein any heterocycle of A¹ and A² can be optionally substituted with -LC(O)R¹⁰;

B is benzothiazolyl, quinolinyl or isoquinolinyl, each of which is optionally substituted with 1-3 R⁶ groups;

X¹, X², X³ and X⁴ are independently CR⁷ or N;

Y is phenyl or a 5-6 membered heteroaryl containing 1-2 heteroatoms selected from N, O and S;

Z is aryl, C_{1-S} heterocycle, or a 5-6 membered heteroaryl containing 1-2 heteroatoms selected from N, O and S; each Y and Z are optionally substituted with 1-3 R⁶ groups;

R¹ and R⁵ are independently H or C₁₋₆ alkyl;

R² and R³ are independently H, C_{1-O} alkyl or halo;

R⁴ is halo, cyano, C₁₋₆alkoxy, or a C₁₋₆ alkyl optionally substituted with halo, alkoxy or amino; R⁶ is hydrogen, halo, C₁₋₆alkoxy, —S(O)₂R¹⁰, —C(O)OR¹⁰, —C(O)R¹⁰, —C(O)NR⁸R⁹, C₁₋₆ alkyl, C₂₋₆ alkenyl or C₂₋₆ alkynyl, each of which can be optionally substituted with halo, amino, hydroxyl, alkoxy or cyano; halo, CN, -L-W, NR⁸R⁹, -L-C(O)R¹⁰, -L-C(O)OR¹⁰, -L-C(O)NR⁸R⁹, OR¹⁰; -L-S(O)₂R¹⁰ Or -L-S(O)₂NR⁸R⁹;

R⁷ is H, halo, C₁₋₆ alkoxy, -L-S(O)₂R¹⁰, C₁₋₆ alkyl optionally substituted with halo, amino, hydroxyl, alkoxy or cyano; NR⁸R⁹, -L-C(O)R¹⁰, -L-C(O)NR⁸R⁹, OR¹⁰; -L-S(O)₂R¹⁰ or -L-S(O)₂NR⁸R⁹;

R⁸ and R⁹ are independently H, -L-W, or C₁₋₆ alkyl, C₂₋₆ alkenyl or C₂₋₆ alkynyl, each of which may be optionally substituted with halo, amino, hydroxyl, alkoxy or cyano; or R⁸ and R⁹ together with the atoms to which they are attached may form a ring;

R¹⁰ is H, -L-W, or C₁₋₆ alkyl, C₂₋₆ alkenyl or C₂₋₆ alkynyl, each of which may be optionally substituted with halo, amino, hydroxyl, alkoxy or cyano;

L is a bond or (CR₂)₁₋₄ wherein R is H or C₁₋₆ alkyl;

W is C₃₋₇cycloalkyl, C₁₋₅heterocycle, aryl or heteroaryl;

m is 0-4;

n is 0-3; and

p is 0-2.

In one embodiment, Compound N has the following structure:

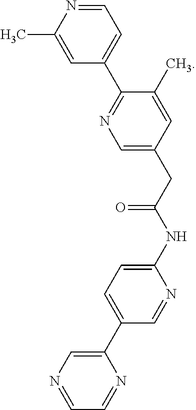 [SMILES: Cc1cc(-c2ncc(CC(=O)Nc3ccc(-c4cnccn4)cn3)cc2C)ccn1]

In one embodiment, Compound N is 2-(2′,3-dimethyl-[2,4′-bipyridin]-5-yl)-N-(5-(pyrazin-2-yl)pyridin-2-yl)acetamide.

In certain embodiments, 2-(2′,3-dimethyl-[2,4′-bipyridin]-5-yl)-N-(5-(pyrazin-2-yl)pyridin-2-yl)acetamide (Compound N) is administered at a dose of about 1 to 50 mg, e.g., about 2 mg to 45 mg, about 3 mg to 40 mg, about 5 mg to 35 mg, 5 mg to 10 mg, or about 10 mg to 30 mg, e.g., about 2 mg, 5 mg, 10 mg, 20 mg, 30 mg, or 40 mg. The dosing schedule can vary from e.g., every other day to daily, twice or three times a day.

In another embodiment, the inhibitor of an immune checkpoint molecule (alone or in combination with one or more other immunomodulators) is used in combination a CDK4/6 inhibitor to treat a disorder, e.g., a disorder described herein (e.g., a disorder disclosed in a publication listed in Table 1). In one embodiment, the CDK4/6 inhibitor is disclosed herein, e.g., in Table 1. In one embodiment, the CDK4/6 inhibitor is 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) as disclosed herein in a publication recited in Table 1. In certain embodiments, the CDK4/6 inhibitor is disclosed in PCT Publication No. WO 2011/101409. In certain embodiments, 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) has the structure provided in Table 1, or in a publication recited in Table 1. In one embodiment, the inhibitor of the immune checkpoint molecule (e.g., one of Nivolumab, Pembrolizumab or MSB0010718C) is used in combination with 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide (Compound O) to treat a disorder described herein, e.g., in a publication recited in Table 1, such as a cancer, a mantle cell lymphoma, a liposarcoma, a non-small cell lung cancer, a melanoma, a squamous cell esophageal cancer, or a breast cancer.

In one embodiment, the CDK4/6 inhibitor is compound according to formula (I)

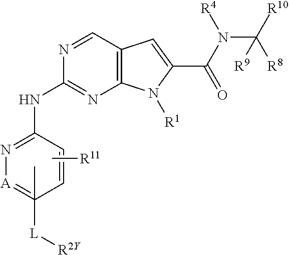

wherein:

R¹ is C₃₋₇ alkylI; C₄₋₇ cycloalkyl optionally substituted with one substituent selected from the group consisting of C₁₋₆ alkyl and OH; phenyl optionally substituted with one substitutent selected from the group consisting of C₁₋₆ alkyl, C(CH₃)₂CN, and OH; piperidinyl optionally substituted with one cyclopropyl or C₁₋₆ alkyl; tetrahydropyranyl optionally substituted with one cyclopropyl or C₁₋₆ alkyl; or bicyclo[2.2.1]heptanyl;

A is CH or N;

R¹¹ is hydrogen or C₁₋₄ alkyl;

L is a bond, C(O), or S(O)2;

R^{2Y} is

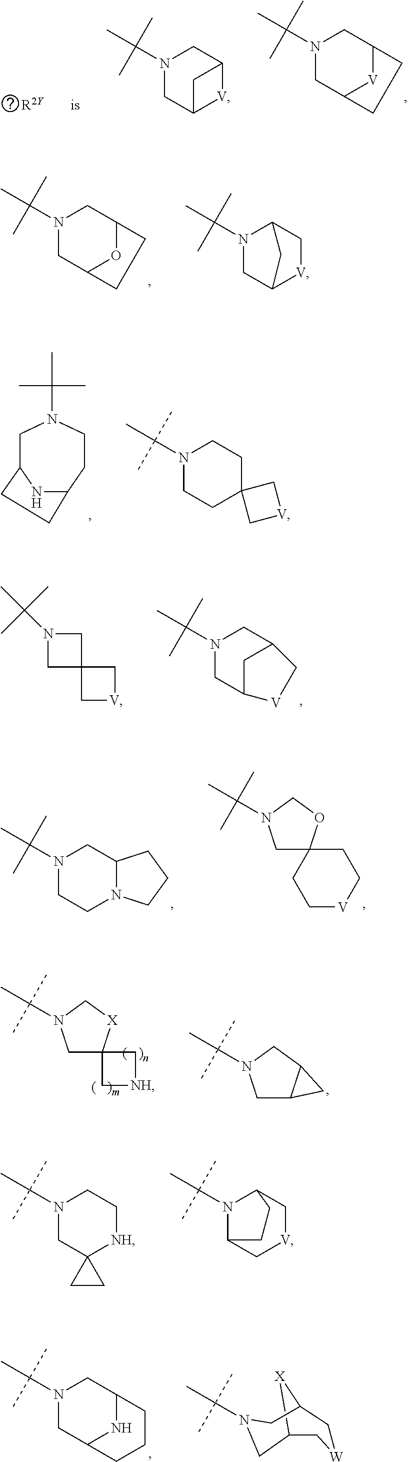

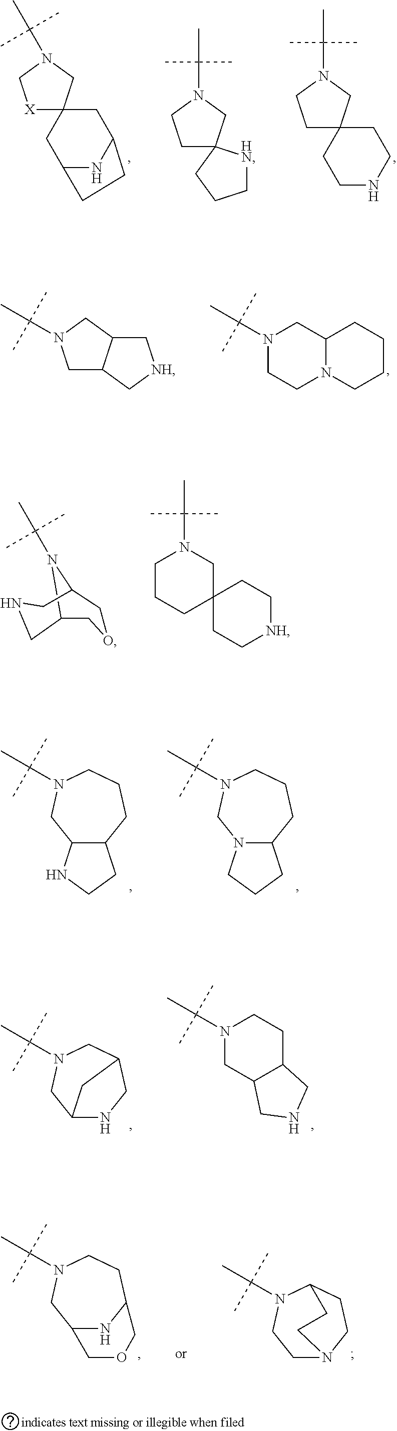

V is NH or CH₂;

X is O or CH₂;

W is O or NH;

m and n are each independently 1, 2, or 3 provided that m and n are not both 3; each R^{2Y} is optionally substituted with one to four substituents each independently selected from the group consisting of: C₁₋₃ alkyl optionally substituted with one or two substituents each independently selected from the group consisting of hydroxy, NH2, and —S—C-₁₋₃ alkyl; CD₃; halo; oxo; C₁₋₃ haloalkyl; hydroxy; NH2; dimethylamino; benzyl; —C(O)—C₁₋₃alkyl optionally substituted with one or two substituents each independently selected from the group consisting of NH_{2′}—SCH₃ and NHC(O)CH₃; —S(O)2-C-₁₋₄alkyl; pyrrolidinyl-C(O)—; and —C(O)2-C₁₋₃alkyl;

R⁴ is hydrogen, deuterium, or C(R⁵)(R⁶)(R⁷); and

R⁵, R⁶, R⁷, R⁸, R⁹ and R¹⁰ are each independently H or deuterium; or a pharmaceutically acceptable salt thereof.

In one embodiment, Compound O has the following structure:

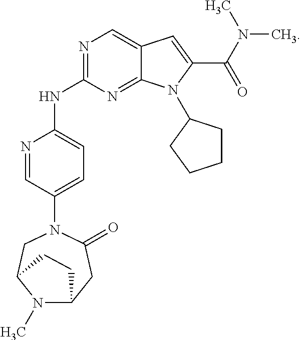 [SMILES: CN(C)C(=O)c1cc2cnc(Nc3ccc(N4C[C@H]5CC[C@@H](CC4=O)N5C)cn3)nc2n1C1CCCC1]

In one embodiment, Compound O is 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide.

In another embodiment, the inhibitor of an immune checkpoint molecule (alone or in combination with one or more other immunomodulators) is used in combination an FGFR2 and/or FGFR4 inhibitor to treat a disorder, e.g., a disorder described herein (e.g., a disorder disclosed in a publication recited in Table 1). In one embodiment, the FGFR2 and/or FGFR4 inhibitor is disclosed herein, e.g., in Table 1. In one embodiment, the FGFR2 and/or FGFR4 inhibitor is an antibody molecule drug conjugate against an FGFR2 and/or FGFR4 (e.g., mAb 12425 or Compound P) disclosed herein, or in a publication disclosed in Table 1. In certain embodiments, the FGFR2 and/or FGFR4 inhibitor is disclosed, e.g., in PCT Publication No. WO 2014/160160. In one embodiment, the FGFR2 and/or FGFR4 inhibitor (e.g., one of Nivolumab, Pembrolizumab or MSB0010718C) is used in combination with an antibody molecule drug conjugate against an FGFR2 and/or FGFR4 (e.g., mAb 12425 or Compound P) to treat a disorder described herein, e.g., in a publication recited in Table 1, such as a cancer, a gastric cancer, a breast cancer, a rhabdomyosarcoma, a liver cancer, an adrenal cancer, a lung cancer, an esophageal cancer, a colon cancer, or an endometrial cancer.

In some embodiments, Compound P is an antibody molecule drug conjugate against an FGFR2 and/or FGFR4, e.g., mAb 12425. In some embodiments, Compound P is an antibody molecule drug conjugate against an FGFR2 and/or FGFR4 that comprises 1, 2, 3, 4, 5, or 6 CDRs according to Kabat or Chothia, a VH and/or VL, of any of the antibodies in Table 1 of WO 2014/160160. In some embodiments, Compound P is an antibody molecule drug conjugate against an FGFR2 and/or FGFR4 that comprises a linker of N-succinimidyl-4-(maleimidomethyl)cyclohexanecarboxylate (SMCC) and a payload of ^{N2′}-deacetyl-N^{2′}-(3-mercapto-1-oxopropyl)-maytansine (DM1). In some embodiments, Compound P is an antibody molecule drug conjugate having the following formula:

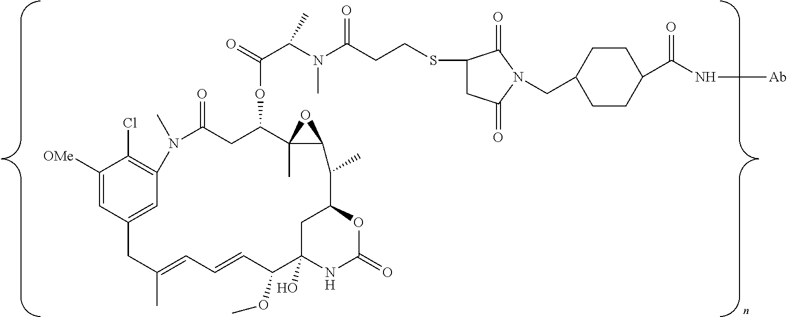

wherein Ab is an antibody or antigen binding fragment thereof comprising a heavy chain CDR1 of SEQ ID NO: 1, 21, 41, 61, 81, or 101, a heavy chain CDR2 of SEQ ID NO: 2, 22, 42, 62, 82, or 102, a heavy chain CDR3 of SEQ ID NO: 3, 23, 43, 63, 83, or 103, and a light chain CDR1 of SEQ ID NO: 11, 31, 51, 71, 91, or 111 a light chain CDR2 of SEQ ID NO: 12, 32, 52, 72, 92, or 112, a light chain CDR3 of SEQ ID NO: 13, 33, 53, 73, 93, or 113, wherein the CDR is defined in accordance with the Kabat definition; e.g., as disclosed in claim 29 of WO 2014/160160.

In another embodiment, the inhibitor of an immune checkpoint molecule (alone or in combination with one or more other immunomodulators) is used in combination an M-CSF inhibitor to treat a disorder, e.g., a disorder described herein (e.g., a disorder disclosed in a publication listed in Table 1). In one embodiment, the M-CSF inhibitor is disclosed herein, e.g., in Table 1. In one embodiment, the M-CSF inhibitor is an antibody molecule or Fab fragment against M-CSF (e.g., Compound Q) disclosed herein, or in a publication recited in Table 1. In certain embodiments, the antibody molecule or Fab fragment against M-CSF (e.g., Compound Q) is disclosed in PCT Publication No. WO 2004/045532. In one embodiment, the inhibitor of the immune checkpoint molecule (e.g., one of Nivolumab, Pembrolizumab or MSB0010718C) is used in combination with the antibody molecule or Fab fragment against M-CSF (e.g., Compound Q) to treat a disorder described herein, e.g., in a publication recited in Table 1, such as a cancer, a prostate cancer, a breast cancer, or pigmented villonodular synovitis (PVNS).

In some embodiments, Compound Q is a monoclonal antibody molecule against M-CSF or a fragment (e.g., Fab fragment) thereof. In some embodiments, Compound Q is a monoclonal antibody or Fab fragment that binds to the same epitope as monoclonal antibody 5H4 (ATCC Accession No. HB10027), e.g., as described in WO 2004/045532. In other embodiments, Compound Q is a monoclonal antibody or Fab fragment thereof that competes with monoclonal antibody 5H4 (ATCC Accession No. HB10027) for binding to M-CSF, e.g., as described in WO 2004/045532. In some embodiments, Compound Q is a monoclonal antibody or Fab fragment that comprises 1, 2, 3, 4, 5 or 6 CDRs of monoclonal antibody 5H4 (ATCC Accession No. HB10027), e.g., as described in WO 2004/045532. In embodiments, the M-CSF inhibitor or Compound Q is administered at an average dose of about 10 mg/kg.

In another embodiment, the inhibitor of an immune checkpoint molecule (alone or in combination with one or more other immunomodulators) is used in combination an inhibitor of one or more of c-KIT, histamine release, Flt3 (e.g., FLK2/STK1) or PKC to treat a disorder, e.g., a disorder described herein (e.g., a disorder disclosed in a publication listed in Table 1). In one embodiment, the inhibitor of one or more of c-KIT, histamine release, Flt3 (e.g., FLK2/STK1) or PKC is disclosed herein, e.g., in Table 1. In one embodiment, the inhibitor of one or more of c-KIT, histamine release, Flt3 (e.g., FLK2/STK1) or PKC is Midostaurin (Compound R) disclosed herein, e.g., in a publication recited in Table 1. In certain embodiments, the inhibitor of one or more of c-KIT, histamine release, Flt3 (e.g., FLK2/STK1) or PKC is disclosed in PCT Publication No. WO 2003/037347, European Patent Application Publication No. EP 1441737, or U.S. Patent Application Publication No. 2012/252785. In one embodiment, Midostaurin (Compound R) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Midostaurin (Compound R) to treat a disorder described herein, e.g., in a publication recited in Table 1, such as a cancer, a colorectal cancer, a myeloid leukemia, myelodysplastic syndrome, an age-related mascular degeration, a diabetic complication, or a dermatologic disorder.

In one embodiment, the inhibitor of one or more of c-KIT, histamine release, Flt3 (e.g., FLK2/STK1) or PKC is a staurosporine derivative of formula,

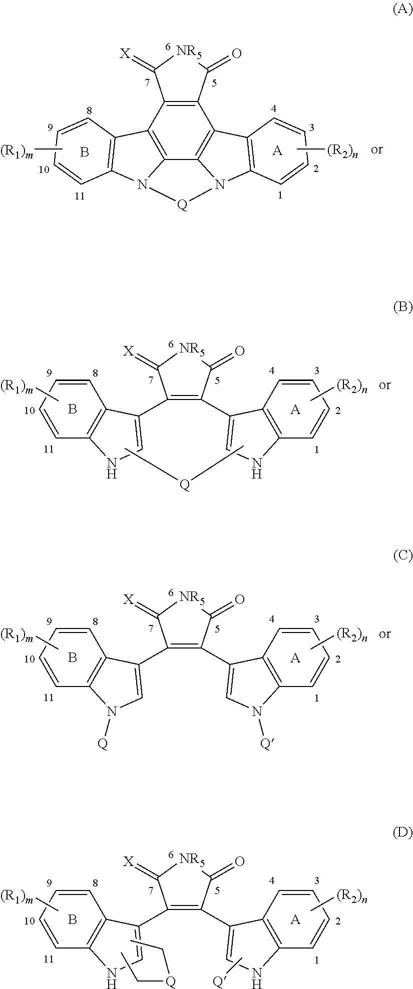

wherein R, and R2 are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;

n and m are, independently of one another, a number from and including 0 to and including 4;

R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;

X stands for 2 hydrogen atoms; for 1 hydrogen atom and hydroxy; for O; or for hydrogen and lower alkoxy;

Q and Q′ are independently a pharmaceutically acceptable organic bond or hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;

or a salt thereof, if at least one salt-forming group is present, or hydrogenated derivative thereof,

In one embodiment, Midostaurin (Compound R) has the following structure:

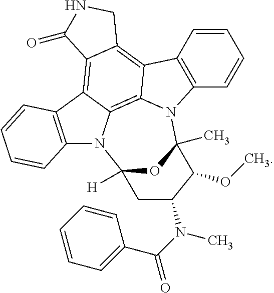 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

In one embodiment, Midostaurin (Compound R) is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide.

In another embodiment, the inhibitor of an immune checkpoint molecule (alone or in combination with one or more other immunomodulators) is used in combination an inhibitor of one or more of VEGFR-2, PDGFRbeta, KIT or Raf kinase C to treat a disorder, e.g., a disorder described herein (e.g., a disorder in a publication listed in Table 1). In one embodiment, the inhibitor of one or more of VEGFR-2, PDGFRbeta, KIT or Raf kinase C is disclosed herein, e.g., in Table 1. In one embodiment, the inhibitor of one or more of VEGFR-2, PDGFRbeta, KIT or Raf kinase C is 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) as disclosed herein, e.g., in a publication recited in Table 1. In certain embodiments, the inhibitor of one or more of VEGFR-2, PDGFRbeta, KIT or Raf kinase C is disclosed, e.g., in PCT Publication No. WO 2007/030377 or U.S. Pat. No. 7,482,367. In one embodiment, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound S) to treat a disorder described herein, e.g., in a publication recited in Table 1, such as a cancer, a melanoma, or a solid tumor.

In one embodiment, the inhibitor of one or more of VEGFR-2, PDGFRbeta, KIT or Raf kinase C is compound of formula (I):

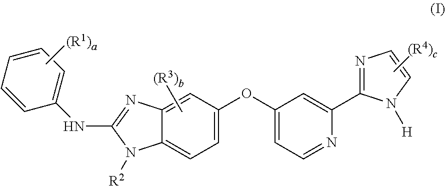

wherein, each R is independently selected from hydroxy, halo, C₁₋₆ alkyl, C₁₋₆ alkoxy, (C₁₋₆ alkyl)sulfanyl, (C₁₋₆alkyl)sulfonyl, cycloalkyl, heterocycloalkyl, phenyl, and heteroaryl; R² is C₁₋₆ alkyl or halo(C₁₋₆ alkyl);

each R³ is independently selected from halo, C₁₋₆ alkyl, and C₁₋₆alkoxy;

each R⁴ is independently selected from hydroxy, C₁₋₆ alkyl, C₁₋₆ alkoxy, halo, carboxyl, (C₁₋₆alkoxy)carbonyl, aminocarbonyl, C₁₋₆ alkylaminocarbonyl, carbonitrile, cycloalkyl, heterocycloalkyl, heterocycloalkylcarbonyl, phenyl, and heteroaryl;

wherein R¹, R², R³, and R⁴ may be optionally substituted with one or more substituents independently selected from hydroxy, halo, C₁₋₆ alkyl, halo(C₁₋₆ alkyl), C₁₋₆ alkoxy, and halo(C₁₋₆alkoxy);

a is 1, 2, 3, 4, or 5;

b is 0, 1, 2, or 3; and

c is 1 or 2;

or a tautomer, stereoisomer, polymorph, ester, metabolite, or prodrug thereof or a pharmaceutically acceptable salt of the compound, tautomer, stereoisomer, polymorph, ester, metabolite, or prodrug.

In one embodiment Compound S has the following structure:

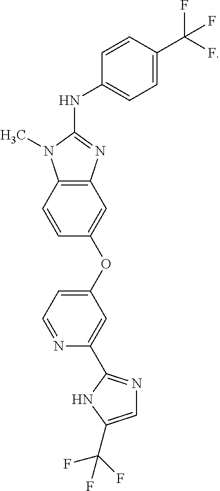 [SMILES: Cn1c(Nc2ccc(C(F)(F)F)cc2)nc2cc(Oc3ccnc(-c4ncc(C(F)(F)F)[nH]4)c3)ccc21]

In one embodiment, Compound S is 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine.

In another embodiment, the inhibitor of an immune checkpoint molecule (alone or in combination with one or more other immunomodulators) is used in combination a somatostatin agonist and/or growth hormone release inhibitor to treat a disorder, e.g., a disorder described herein (e.g., a disorder disclosed in a publication listed in Table 1). In one embodiment, the somatostatin agonist and/or growth hormone release inhibitor is disclosed herein, e.g., in Table 1. In one embodiment, the somatostatin agonist and/or growth hormone release inhibitor is Pasireotide diaspartate (Compound T) disclosed herein, e.g., in a publication recited in Table 1. In certain embodiments, the somatostatin agonist and/or growth hormone release inhibitor is disclosed, e.g., in PCT Publication No. WO2002/010192 or U.S. Pat. No. 7,473,761. In one embodiment, Pasireotide diaspartate (Compound T) has the structure provided in Table 1, or in a publication recited in Table 1. In one embodiment, the inhibitor of the immune checkpoint molecule (e.g., one of Nivolumab, Pembrolizumab or MSB0010718C) is used in combination with Pasireotide diaspartate (Compound T) to treat a disorder described herein, e.g., in a publication recited in Table 1, such as a cancer, a prostate cancer, an endocrine cancer, a nurologic cancer, a skin cancer (e.g., a melanoma), a pancreatic cancer, a liver cancer, Cushing's syndrome, a gastrointestinal disorder, acromegaly, a liver and biliary tract disorder, or liver cirrhosis.

In one embodiment, Pasireotide diaspartate (Compound T) has the following structure:

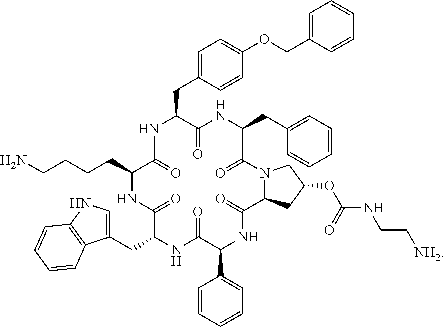 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O]

In one embodiment, Pasireotide diaspartate (Compound T) is cyclo((4R)-4-(2-aminoethylcarbamoyloxy)-L-prolyl-L-phenylglycyl-D-tryptophyl-L-lysyl-4-O-benzyl-L-tyrosyl-L-phenylalanyl).

In another embodiment, the inhibitor of an immune checkpoint molecule (alone or in combination with one or more other immunomodulators) is used in combination a signal transduction modulator and/or angiogenesis inhibitor to treat a disorder, e.g., a disorder described herein (e.g., a disorder disclosed in a publication listed in Table 1). In one embodiment, the signal transduction modulator and/or angiogenesis inhibitor is disclosed herein, e.g., in Table 1.

In one embodiment, the signal transduction modulator and/or angiogenesis inhibitor is Dovitinib (Compound U) as disclosed herein, or in a publication recited in Table 1. In certain embodiments, the signal transduction modulator and/or angiogenesis inhibitor is disclosed, e.g., in PCT Publication No. WO 2009/115562 or U.S. Pat. No. 8,563,556. In one embodiment, Dovitinib (Compound U) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Dovitinib (Compound U) to treat a disorder described herein, e.g., in a publication recited in Table 1, such as a cancer, a respiratory/thoracic cancer, a multiple myeloma, a prostate cancer, a non-small cell lung cancer, an endocrine cancer, or a neurological genetic disorder.

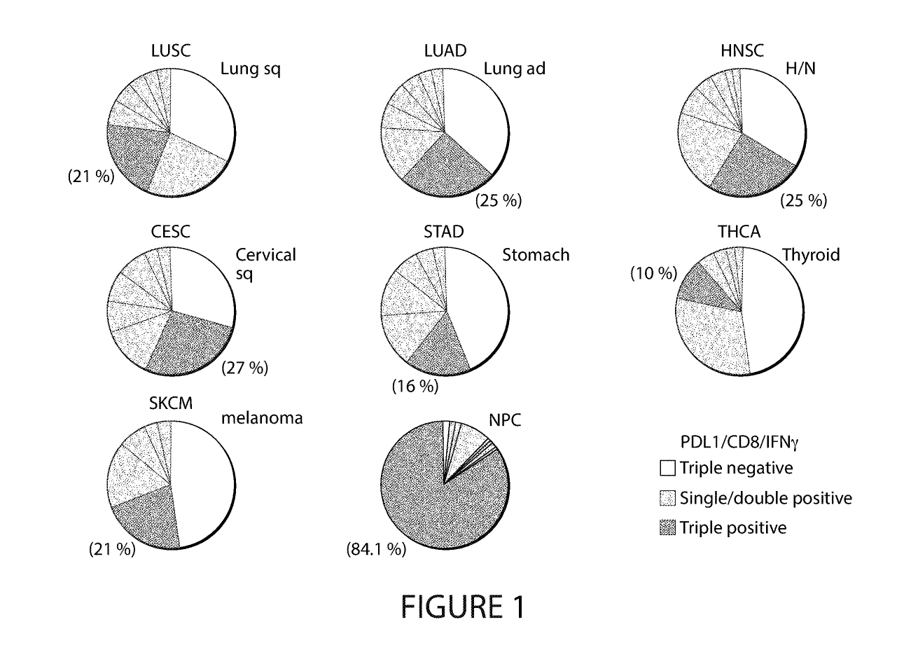

In one embodiment, the signal transduction modulator and/or angiogenesis inhibitor is a substantially pure crystalline anhydrous form of 1-amino-5-fluoro-3-[5-(4-methylpiperazin-1-yl)-1H-benzimidazol-2-yl]quinolin-2(1H)-one lactic acid salt characterized by an x-ray powder diffraction pattern that shows a characteristic maxima at 8.2, 18.5 degrees, 2 theta. In one embodiment, the signal transduction modulator and/or angiogenesis inhibitor is a substantially pure crystalline anhydrous form II of 1-amino-5-fluoro-3-[5-(4-methylpiperazin-1-yl)-1H-benzimidazol-2-yl]quinolin-2(1H)-one lactic acid salt characterized by the x-ray powder diffraction pattern shown in FIG. 1 of WO 2009/115562.

In one embodiment, Dovitinib (Compound U) has the following structure:

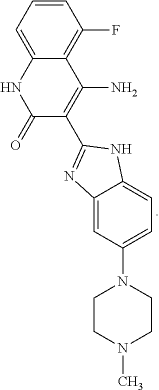 [SMILES: CN1CCN(c2ccc3[nH]c(-c4c(N)c5c(F)cccc5[nH]c4=O)nc3c2)CC1]

In one embodiment, Dovitinib (Compound U) is 1-amino-5-fluoro-3-[6-(4-methyl-1-piperazinyl)-1H-benzimidazol-2-yl]-2(1H)-quinolinone.

In another embodiment, the inhibitor of an immune checkpoint molecule (alone or in combination with one or more other immunomodulators) is used in combination an ALK inhibitor to treat a disorder, e.g., a disorder described herein (e.g., a disorder disclosed in a publication listed in Table 1). In one embodiment, the ALK inhibitor is disclosed herein, e.g., in Table 1. In one embodiment, the ALK inhibitor is N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) as disclosed herein, or in a publication recited in Table 1. In certain embodiments, the ALK inhibitor is disclosed in PCT Publication No. WO 2008/073687 or U.S. Pat. No. 8,372,858. In one embodiment, N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) has the structure provided in Table 1, or as disclosed in a publication recited in Table 1. In one embodiment, the inhibitior of the immune checkpoint molecule (e.g., one of Nivolumab, Pembrolizumab or MSB0010718C) is used in combination with N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine (Compound W) to treat a disorder described herein, e.g., in a publication recited in Table 1, such as a cancer, an anaplastic large-cell lymphoma (ALCL), a non-small cell lung carcinoma (NSCLC), or a neuroblastoma.

In one embodiment, the ALK inhibitor is a compound having Formula (1):

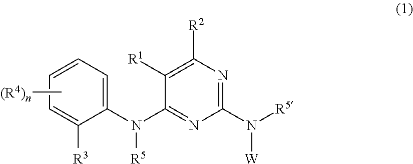

or pharmaceutically acceptable salts thereof; wherein

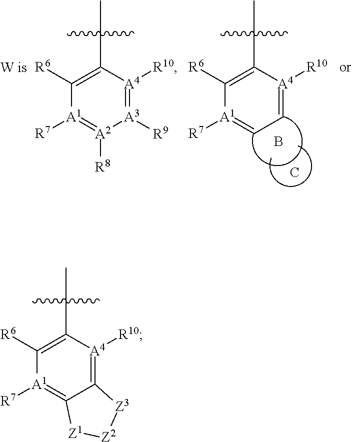

A¹ and A⁴ are independently C or N; each A² and A³ is C, or one of A² and A³ is N when R⁶ and R⁷ form a ring;

B and C are independently an optionally substituted 5-7 membered carbocyclic ring, aryl, heteroaryl or heterocyclic ring containing N, O or S;

Z¹, Z² and Z³ are independently NR¹¹, C═O, CR—OR, (CR₂)_{I-2} or ═C—R¹²;

R¹ and R² are independently halo, OR¹², NR(R¹²), SR¹², or an optionally substituted C₁₋₆ alkyl, C₂-6 alkenyl or C₂-6 alkynyl; or one of R¹ and R² is H;

R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;

R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;
R, R⁵ and R^{5′} are independently H or C₁₋₆ alkyl;
R⁸ and R⁹ are independently C₁₋₆ alkyl, C₂₋₆ alkenyl, C₂₋₆alkynyl, halo or X, or one of R⁸ and R⁹ is H when R¹ and R² form a ring; and provided one of R⁸ and R⁹ is X;

alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N; R¹¹ is H, C₁₋₆ alkyl, C₂₋₆ alkenyl, (CR₂)_{P}CO₁₋₂R, (CR₂)_{P}OR, (CR₂)_{P}R¹³ (CR₂)_{P}NRR¹², (CR₂)_{P}CONRR¹² or (CR₂)ₚSO₁₋₂R¹²;

R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;

X is (CR₂)_{q}Y, cyano, CO₁₋₂R¹², CONR(R¹²), CONR(CR₂)_{P}NR(R¹²), CONR(CR₂)_{P}OR¹², CONR(CR₂)_{P}SR¹², CONR(CR₂)_{P}S(O)₁₋₂R¹² or (CR₂)₁₋₆NR(CR₂)ₚOR¹²;

Y is an optionally substituted 3-12 membered carbocyclic ring, a 5-12 membered aryl, or a 5-12 membered heteroaryl or heterocyclic ring comprising N, O and/or S and attached to A or A³ or both via a carbon atom of said heteroaryl or heterocyclic ring when q in (CR₂)_{q}Y is 0; and n, p and q are independently 0-4.

In one embodiment, Compound W has the following structure:

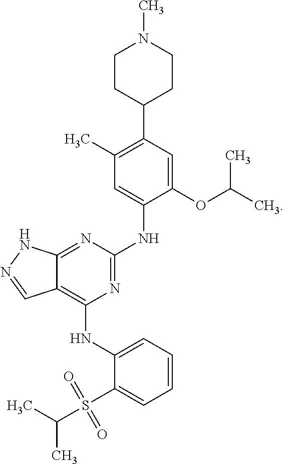 [SMILES: Cc1cc(Nc2nc(Nc3ccccc3S(=O)(=O)C(C)C)c3cn[nH]c3n2)c(OC(C)C)cc1C1CCN(C)CC1]

In one embodiment, Compound W is N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine.

In another embodiment, the inhibitor of an immune checkpoint molecule (alone or in combination with one or more other immunomodulators) is used in combination an IGF-1R inhibitor to treat a disorder, e.g., a disorder described (e.g., a disorder disclosed in a publication listed in Table 1). In one embodiment, the IGF-1R inhibitor is disclosed herein, e.g., in a publication recited in Table 1. In one embodiment, the IGF-1R inhibitor is 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N²-(4-(1-ethylpiperidin-4-yl)-2-fluoro-5-methylphenyl)-N⁴-(5-methyl-1H-pyrazol-3-yl)pyrimidine-2,4-diamine (Compound Z), as disclosed herein, or in a publication recited in Table 1. In certain embodiments, the IGF-1R inhibitor is disclosed, e.g., in PCT Publication No. WO 2010/002655 or U.S. Pat. No. 8,519,129. In one embodiment, 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N²-(4-(1-ethylpiperidin-4-yl)-2-fluoro-5-methylphenyl)-N⁴-(5-methyl-1H-pyrazol-3-yl)pyrimidine-2,4-diamine (Compound Z) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide (Compound X), 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine (Compound Y), 5-chloro-N²-(4-(1-ethylpiperidin-4-yl)-2-fluoro-5-methylphenyl)-N⁴-(5-methyl-1H-pyrazol-3-yl)pyrimidine-2,4-diamine (Compound Z) to treat a disorder described herein, e.g., in a publication recited in Table 1, such as a cancer or a sarcoma.

In one embodiment, the IGF-1R inhibitor is compound of Formula (1):

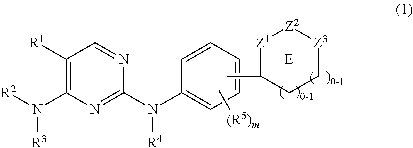

or a physiologically acceptable salt thereof; wherein ring E may optionally contain a double bond; one of Z¹, Z² and Z³ is NR⁶, N(R⁶)—O or S(O)₁₋₂ and the others are CR₂;

R¹ is halo or an optionally halogenated C₁₋₆ alkyl;

R² is pyridine-2-onyl, azepan-2-onyl or a monocyclic 5-6 membered heteroaryl having 1-3 heteroatoms selected from N, O and S; each of which is optionally substituted with R⁹ wherein R⁹ is C₁₋₆ alkyl, C₁₋₆ haloalkyl or C₃₋₇ cycloalkyl;

R³ and R⁴ are each H;

R⁵ is halo, hydroxyl, C₁₋₆ alkyl, C₁—O alkoxy, halo-substituted C₁₋₆ alkyl, halo-substituted C₁₋₆ alkoxy, cyano or C(O)O₀₋₁R⁸;

R⁶ is H; C₁₋₆ alkyl, C₂-C₆ alkenyl or C₂-C₆ alkynyl, each of which may be optionally substituted with halo and/or hydroxyl groups; —(CR₂)_{P}—OR⁷, —(CR₂)_{P}—CH(OH)C₁F₂ₜ₊₁ wherein t is 1-3, (CR₂)_{P}—CN; (CR₂)_{P}—NR(R⁷), —(CR₂)_{P}—C(O)OR⁷, (CR₂)_{P}NR(CR₂)_{P}OR⁷, (CR₂)_{P}NR-L-C(O)R⁸, C(O)(CR₂)_{q}OR⁸, —C(O)O—(CR₂)_{P}—NRR⁷, —C(O)—(CR₂)_{P}—OR⁷, L-Y, -L-C(O)R⁷, -L-C(O)—NRR⁷, -L-C(O)—NR—(CR₂)_{P}-NRRVL-C(O)NR(CR₂)_{P}OR⁷, -L-C(O)—(CR₂)—NR—C(O)—R⁸, -L-C(O)NR(CR₂)pSR⁷, -L-C(O)NR(CR₂)_{P}S(O)₁₋₂R⁸, -L-S(O)₂R⁸, -L-S(O)₂—(CR₂)_{q}—NRR⁷, -L-S(O)₂NR(CR₂)_{P}NR(R⁷) or -L-S(O)₂NR(CR₂)_{P}OR⁷;

alternatively, R⁶ is a radical selected from formula (a), (b), (c) or (d):

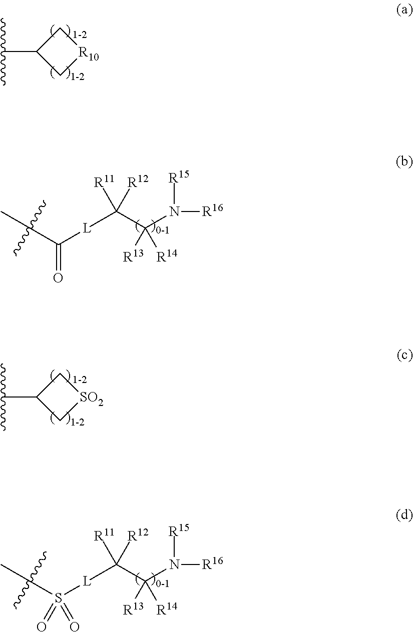

R¹⁰ is O, S, NR¹⁷ wherein R¹⁷ is H, C₁₋₆ alkyl, SO₂R^{8a} or CO₂R^{8a};

R¹¹, R¹², R¹³, R¹⁴, R¹⁵ and R¹⁶ are independently selected from H; C₁₋₆ alkoxy; C₁₋₆ alkyl, C₂₋₆ alkenyl or C₂₋₆alkynyl, each of which may be optionally substituted with halo, amino or hydroxyl groups; or R¹¹ and R¹², R¹² and R¹⁵, R¹⁵ and R¹⁶, R¹³ and R¹⁴, or R¹³ and R¹⁵ together with the atoms to which they are attached may form a 3-7 membered saturated, unsaturated or partially unsaturated ring containing 1-3 heteroatoms selected from N, O and S, and optionally substituted with oxo and 1-3 R⁵ groups;

L is (CR₂)₁₋₄ or a bond;

Y is C₃₋₇ carbocyclic ring, C₆₋₁₀ aryl, or a 5-10 membered heteroaryl or 4-10 membered heterocyclic ring, each of which is optionally substituted with 1-3 R⁵ groups;

R⁷, R⁸ and R^{8a} are independently C₁₋₆ alkyl, C₂₋₆ alkenyl or C₂₋₆ alkynyl, each of which may be optionally substituted with halo, amino, hydroxyl or cyano; (CR₂)_{q}Y or C₁₋₆ alkoxy; or R⁷ is H;

each R is independently H or C₁₋₆alkyl;

R and R⁷ together with N in each NRR⁷ may form a 5-6 membered ring containing 1-3 heteroatoms selected from N, O and S, and optionally substituted with oxo and 1-3 R⁵ groups;

m is 2-4;

n is 1-3;

p is 1-4; and

q is 0-4.

In one embodiment, Compound X has the following structure:

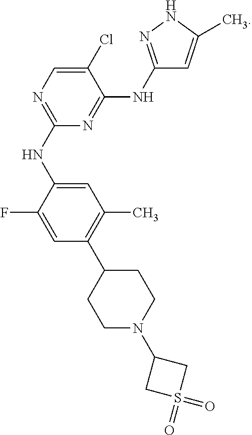 [SMILES: Cc1cc(Nc2nc(Nc3cc(C)c(C4CCN(C5CS(=O)(=O)C5)CC4)cc3F)ncc2Cl)n[nH]1]

In one embodiment, Compound X is 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide.

In one embodiment, Compound Y has the following structure:

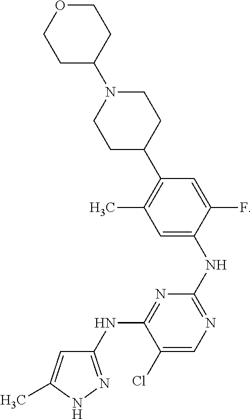 [SMILES: Cc1cc(Nc2nc(Nc3cc(C)c(C4CCN(C5CCOCC5)CC4)cc3F)ncc2Cl)n[nH]1]

In one embodiment, Compound Y is 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine.

In one embodiment, Compound Z has the following structure:

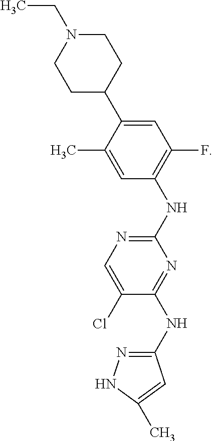 [SMILES: CCN1CCC(c2cc(F)c(Nc3ncc(Cl)c(Nc4cc(C)[nH]n4)n3)cc2C)CC1]

In one embodiment, Compound Z is 5-chloro-N2-(4-(1-ethylpiperidin-4-yl)-2-fluoro-5-methylphenyl)-N⁴-(5-methyl-1H-pyrazol-3-yl)pyrimidine-2,4-diamine.

In another embodiment, the inhibitor of an immune checkpoint molecule (alone or in combination with one or more other immunomodulators) is used in combination a P-Glycoprotein 1 inhibitor to treat a disorder, e.g., a disorder described herein (e.g., a disorder disclosed in a publication listed in Table 1). In one embodiment, the P-Glycoprotein 1 inhibitor is disclosed herein, e.g., in Table 1. In one embodiment, the P-Glycoprotein 1 inhibitor is Valspodar (Compound AA) as disclosed herein, or in a publication recited in Table 1. In certain embodiments, the P-Glycoprotein 1 inhibitor is disclosed, e.g., in EP 296122. In one embodiment, Valspodar (Compound AA) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Valspodar (Compound AA) to treat a disorder described herein, e.g., in a publication recited in Table 1, such as a cancer or a drug-resistant tumor.

In one embodiment, the P-Glycoprotein 1 inhibitor is A cyclosporin

(i) of formula II

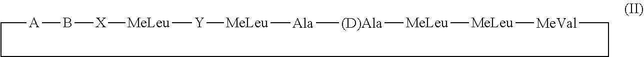

wherein

A is -3′-O-acetyl-MeBmt-

B is -αAbu-, -Thr-, -Val- or Nva-; and

when B is -αAbu-, X is -(D) Ala- and Y is -Val-;

when B is -Thr- or -Val-, X is -Sar- and Y is -Val-; or

when B is -Nva-, X is -Sar- and Y is -Val-, or X is -(D)Ala- and Y is -Val-; or

wherein

A is -3′-O-acetyl-dihydro-MeBmt- or -cis-MeBmt-, B is -αAbu-, X is -Sar- and Y is -Val-: or ii of formula II′

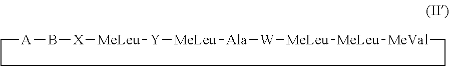

wherein

A is -3′-O-acyl-MeBmt-or-3′-O-acyl-dihydro-MeBmt-residue,

B is -αAbu-, -Thr-, -Val-, -Nva-, or the residue of a β-O-acyl-α-amino acid,

X is -Sar- or the residue of an optically active α-N-methylated α-amino acid residue having the (D)-configuration,

Y is -Val- or additionally, when B is -Nva-, -Nva-, and

W is the residue of a β-hydroxy- or β-O-acyl-α-amino acid having the (D)-configuration; or

iii) wherein the residue at the position 1-position is an -8′-C₁₋₈alkoxy-cis-MeBmt-or-dihydro-MeBmt-or-3′-O-acyl-8′-C₁₋₈alkoxy-cis-MeBmt-or-dihydro-MeBmt-residue; a-3′-O-acyl-cis-MeBmt-residue; a-7′-desmethyl-7′-hydrocarbyl-MeBmt-or-cis-MeBmt-or-3′-O-acyl-7′-desmethyl-7′-hydrocarbyl-MeBmt-or-cis-MeBmt-residue wherein the hydrocarbyl moiety comprises at least two carbon atoms; or a-7′-desmethyl-7′-hydrocarbyl-dihydro-MeBmt-or-3′-O-acetyl-7′-desmethyl-7′-hydrocarbyl-dihydro-MeBmt-residue wherein the hydrocarbyl moiety comprises at least two carbon atoms and wherein any aliphatic group or moiety as or comprising said hydrocarbyl moiety is saturated; or

(iv) wherein the 3′-carbon atom of the residue at the 1-position is oxo, C₁₋₄alkoxyimino, azidoalkylcarbonyloxy or alkoxycarbonyloxy substituted, or wherein the β-carbon atom of the residue at the 2-position is β-oxo substituted or the residue at the 2-position is an (L)-isoleucyl residue; or

(v) of formula XI

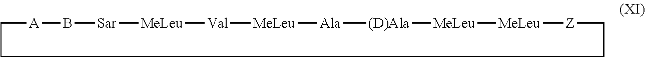

wherein

A is —N-desmethyl-dihydro-MeBmt, B is -Thr- and Z is -MeVal-, or

A is -dihydro-MeBmt-, B is -Thr- and Z is -Val-, or

A is -MeLeu-, B is -αAbu- and Z is -Val-; or which is

(vi) a dicarboxylic acid di-ester of a cyclosporin having a β-hydroxy-(L)-α-amino acid residue at the 2-position.

In one embodiment, Valspodar (Compound AA) has the following structure:

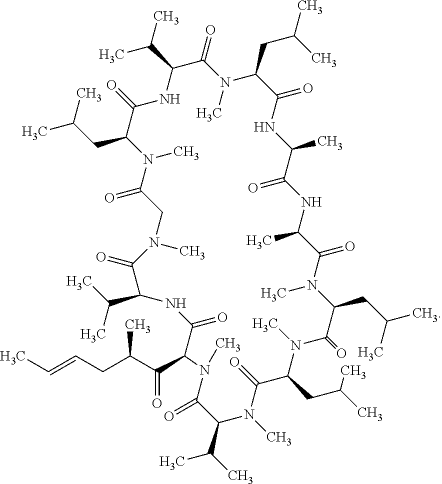 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In one embodiment, Valspodar (Compound AA) is (3S,6S,9S,12R,15S,18S,21S,24S,30S,33S)-6,9,18,24-tetraisobutyl-3,21,30-triisopropyl-1,4,7,10,12,15,19,25,28-nonamethyl-33-[(2R,4E)-2-methyl-4-hexenoyl]-1,4,7,10,13,16,19,22,25,28,31-undecaazacyclotritriacontane-2,5,8,11,14,17,20,23,26,29,32-undecone.

In another embodiment, the inhibitor of an immune checkpoint molecule (alone or in combination with one or more other immunomodulators) is used in combination one or more of a VEGFR inhibitor to treat a disorder, e.g., a disorder described herein (e.g., a disorder disclosed in a publication listed in Table 1). In one embodiment, the VEGFR inhibitor is disclosed herein, e.g., in Table 1. In one embodiment, the VEGFR inhibitor is Vatalanib succinate (Compound BB) as disclosed herein, or in a publication recited in Table 1. In certain embodiments, the VEGFR inhibitor is disclosed, e.g., in WO 98/35958. In one embodiment, Vatalanib succinate (Compound BB) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with Vatalanib succinate (Compound BB) to treat a disorder described herein, e.g., in a publication recited in Table 1, such as a cancer.

In one embodiment, the VEGFR inhibitor is a compound of formula (I),

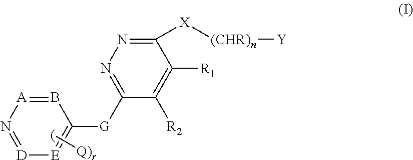

wherein r is 0 to 2, n is 0 to 2, m is 0 to

R1 and R2 (i) are lower alkyl or

(ii) together form a bridge in subformula I*

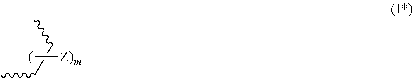

in the binding being achieved via the two terminal carbon atoms, or

(iii) together form a bridge in subformula I**

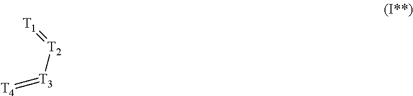

wherein one or two of the ring members T1, T2, T3 and T4 are nitrogen, and the others are in each case CH, and the binding is achieved via T1 and T4

A, B, D, and E are, independently of one another, N or CH, with the stipulation that not more than 2 of these radicals are N;

G is lower alkylene, lower alkylene substituted by acyloxy or hydroxy, —CH2-0-, —CH2-S—, —CH2-NH—, oxa (—0-), thia (—S—), or imino (—NH—); 0 is lower alkyl; R is H or lower alkyl;

X is imino, oxa, or thia;

Y is aryl, pyridyl, or unsubstituted or substituted cycloalkyl; and

Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present; and

wherein the bonds characterized, if present, by a wavy line are either single or double bonds;

or an N-oxide of the defined compound, wherein 1 or more N atoms carry an oxygen atom;

with the stipulation that, if Y is pyridyl or unsubstituted cycloalkyl, X is imino, and the remaining radicals are as defined, G is selected from the group comprising lower alkylene, —CH2-0-, —CH2-S—, oxa and thia; or a salt thereof.

In one embodiment, Vatalanib succinate (Compound BB) has the following structure:

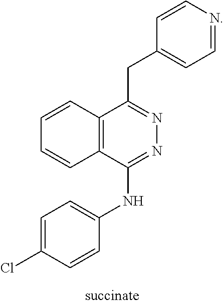 [SMILES: Clc1ccc(Nc2nnc(Cc3ccncc3)c3ccccc23)cc1]

In one embodiment, Vatalanib succinate (Compound BB) is N-(4-Chlorophenyl)-4-(4-pyridinylmethyl)-1-phthalazinamine succinate.

In another embodiment, the inhibitor of an immune checkpoint molecule (alone or in combination with one or more other immunomodulators is used in combination with an IDH inhibitor to treat a disorder, e.g., a disorder described herein (e.g., a disorder disclosed in a publication listed in Table 1). In one embodiment, the IDH inhibitor is disclosed herein, e.g., in Table 1. In one embodiment, the IDH inhibitor is Compound CC as disclosed in Table 1, or in a publication recited in Table 1. In one embodiment, the IDH inhibitor is disclosed, e.g., in PCT Publication No. WO2014/141104. In one embodiment, the inhibitor of the immune checkpoint molecule (e.g., one of Nivolumab, Pembrolizumab or MSB0010718C) is used in combination with Compound CC to treat a disorder described herein, e.g., in a publication recited in Table 1, such as a cancer.

In another embodiment, the inhibitor of an immune checkpoint molecule (alone or in combination with one or more other immunomodulators) is used in combination with a BCL-ABL inhibitor to treat a disorder, e.g., a disorder described herein (e.g., a disorder disclosed in a publication listed in Table 1). In one embodiment, the BCL-ABL inhibitor is disclosed herein, e.g., in Table 1. In one embodiment, the BCL-ABL inhibitor is (R)—N-(4-(chlorodifluoromethoxy)phenyl)-6-(3-hydroxypyrrolidin-1-yl)-5-(1H-pyrazol-5-yl)nicotinamide; (Compound DD) as disclosed in Table 1, or in a publication recited in Table 1. In certain embodiments, (R)—N-(4-(chlorodifluoromethoxy)phenyl)-6-(3-hydroxypyrrolidin-1-yl)-5-(1H-pyrazol-5-yl)nicotinamide (Compound DD) is disclosed, e.g., in PCT Publication No. WO2013/171639, WO2013/171640, WO2013/171641, or WO2013/171642. In one embodiment, (R)—N-(4-(chlorodifluoromethoxy)phenyl)-6-(3-hydroxypyrrolidin-1-yl)-5-(1H-pyrazol-5-yl)nicotinamide (Compound DD) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with (R)—N-(4-(chlorodifluoromethoxy)phenyl)-6-(3-hydroxypyrrolidin-1-yl)-5-(1H-pyrazol-5-yl)nicotinamide (Compound DD) to treat a disorder described herein, e.g., in a publication recited in Table 1, such as a cancer.

In one embodiment, the BCL-ABL inhibitor is a compound of formula (I):

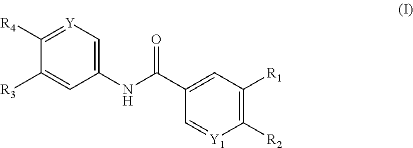

in which:

Ri is pyrazolyl; wherein said pyrazolyl is unsubstituted or substituted with 1 to 2 R₆ groups; R₂ is pyrrolidinyl; wherein said pyrrolidinyl is substituted with one R7 group;

R3 is selected from hydrogen and halo;

R4 is selected from —SF₅ and —Y2-CF2-Y3;

Re at each occurrence is independently selected from hydrogen, hydroxy, methyl, methoxy, cyano, trifluoromethyl, hydroxy-methyl, halo, amino, fluoro-ethyl, ethyl and cyclopropyl;

R₇ is selected from hydroxy, methyl, halo, methoxy, hydroxy-methyl, amino, methylamino, amino-methyl, trifluoromethyl, 2-hydroxypropan-2-yl, methyl-carbonyl-amino, dimethyl-amino, 2-amino-3-methylbutanoyl)oxy, carboxy, methoxy-carbonyl, phosphonooxy, cyano and amino-carbonyl;

Y is selected from CH and N;

Yi is selected from CH and N;

Y₂ is selected from CF₂, O and S(O)₀₋₂; and

Y₃ is selected from hydrogen, chloro, fluoro, methyl, difluoromethyl and trifluoromethyl; or the pharmaceutically acceptable salts thereof.

In one embodiment, the BCL-ABL inhibitor has the following structure:

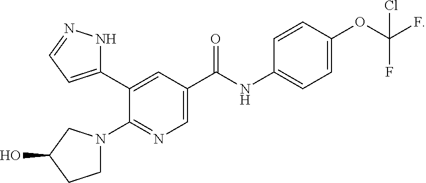 [SMILES: O=C(Nc1ccc(OC(F)(F)Cl)cc1)c1cnc(N2CC[C@@H](O)C2)c(-c2ccn[nH]2)c1]

In one embodiments, the BCL-ABL inhibitor is (R)—N-(4-(chlorodifluoromethoxy)phenyl)-6-(3-hydroxypyrrolidin-1-yl)-5-(1H-pyrazol-5-yl)nicotinamide (Compound DD).

In another embodiment, the inhibitor of an immune checkpoint molecule (alone or in combination with one or more other immunomodulators) is used in combination with a c-RAF inhibitor to treat a disorder, e.g., a disorder described herein (e.g., a disorder disclosed in a publication listed in Table 1). In one embodiment, the c-RAF inhibitor is disclosed herein, e.g., in Table 1. In one embodiment, the c-RAF inhibitor is Compound EE as disclosed herein, or in a publication recited in Table 1. In certain embodiments, Compound EE is disclosed in PCT Publication No. WO2014/151616. In one embodiment, the inhibitor of the immune checkpoint molecule (e.g., one of Nivolumab, Pembrolizumab or MSB0010718C) is used in combination with Compound EE to treat a disorder described herein, e.g., in a publication recited in Table 1, such as a cancer.

In another embodiment, the inhibitor of an immune checkpoint molecule (alone or in combination with one or more other immunomodulators) is used in combination with an ERK1/2 ATP competitive inhibitor to treat a disorder, e.g., a disorder described herein (e.g., a disorder disclosed in a publication listed in Table 1). In one embodiment, the ERK1/2 ATP competitive inhibitor is disclosed herein, e.g., in Table 1. In one embodiment, the ERK1/2 ATP competitive inhibitor is Compound FF as disclosed herein, or in a publication recited in Table 1. In certain embodiments, Compound FF is disclosed in International Patent Application No. PCT/US2014/062913. In one embodiment, the inhibitor of the immune checkpoint molecule (e.g., one of Nivolumab, Pembrolizumab or MSB0010718C) is used in combination with Compound FF to treat a disorder described herein, e.g., in a publication recited in Table 1, such as a cancer.

In another embodiment, the inhibitor of an immune checkpoint molecule (alone or in combination with one or more other immunomodulators) is used in combination a CSF-1R tyrosine kinase inhibitor to treat a disorder, e.g., a disorder described herein (e.g., a disorder disclosed in a publication listed in Table 1). In one embodiment, the CSF-1R tyrosine kinase inhibitor is disclosed herein, e.g., in Table 1. In one embodiment, the CSF-1R tyrosine kinase inhibitor is 4-((2-(((1R,2R)-2-hydroxycyclohexyl)amino)benzo[d]thiazol-6-yl)oxy)-N-methylpicolinamide (Compound GG) as disclosed herein, or in a publication recited in Table 1. In certain embodiments, the CSF-1R tyrosine kinase inhibitor is disclosed, e.g., in PCT Publication No. WO2005/073224. In one embodiment, 4-((2-(((1R,2R)-2-hydroxycyclohexyl)amino)benzo[d]thiazol-6-yl)oxy)-N-methylpicolinamide (Compound GG) has the structure provided in Table 1, or as disclosed in a publication recited in Table 1. In one embodiment, the inhibitor of the immune checkpoint molecule (e.g., one of Nivolumab, Pembrolizumab or MSB0010718C) is used in combination with 4-((2-(((1R,2R)-2-hydroxycyclohexyl)amino)benzo[d]thiazol-6-yl)oxy)-N-methylpicolinamide (Compound GG) to treat a disorder described herein, e.g., in a publication recited in Table 1, such as a cancer.

In one embodiment, the CSF-1R tyrosine kinase inhibitor is a compound of Formula I′

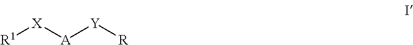

wherein R is selected from

a) substituted or unsubstituted aryl,

b) substituted or unsubstituted heterocyclyl,

c) substituted or unsubstituted cycloalkyl,

d) substituted or unsubstituted cycloalkenyl,

e) H,

f) substituted or unsubstituted alkyl,

g) substituted or unsubstituted alkenyl,

h) substituted or unsubstituted alkynyl,

i) alkylaminocarbonyl,

j) aminocarbonyl, and

k) cyano;

wherein R¹ is

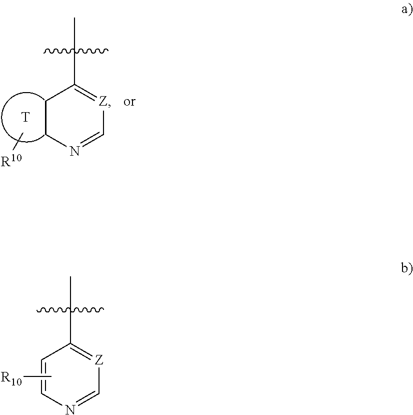

wherein ring T is selected from phenyl and 5-6-membered heteroaryl; wherein Z is selected from N or CR″; wherein R^{x} is selected from H, CN, NH₂, F, alkylcarbonylamino, and alkylaminocarbonyl; wherein R¹⁰ is one or more substituents selected from C₁₋₆-alkoxy, C₁₋₆-haloalkoxy, C₁₋₆-alkylamino-C₁₋₆-alkoxy, aryl-C₁₋₆-alkoxy, heterocyclyl-C₁₋₆-alkoxy, cycloalkyl-C₁₋₆-alkoxy, heterocyclyl-C₁₋₆-(hydroxyalkoxy), cycloalkyl-C₁₋₆-(hydroxyalkoxy), aryl-C₁₋₆-(hydroxyalkoxy), C₁₋₆-alkoxyalkoxy, aryloxy-C₁₋₆-alkoxy, heterocyclyloxy-C₁₋₆-alkoxy, cycloalkyloxy-C₁₋₆-alkoxy, aryloxy, heterocyclyloxy, and cycloalkyloxy;

wherein A is selected from the following:

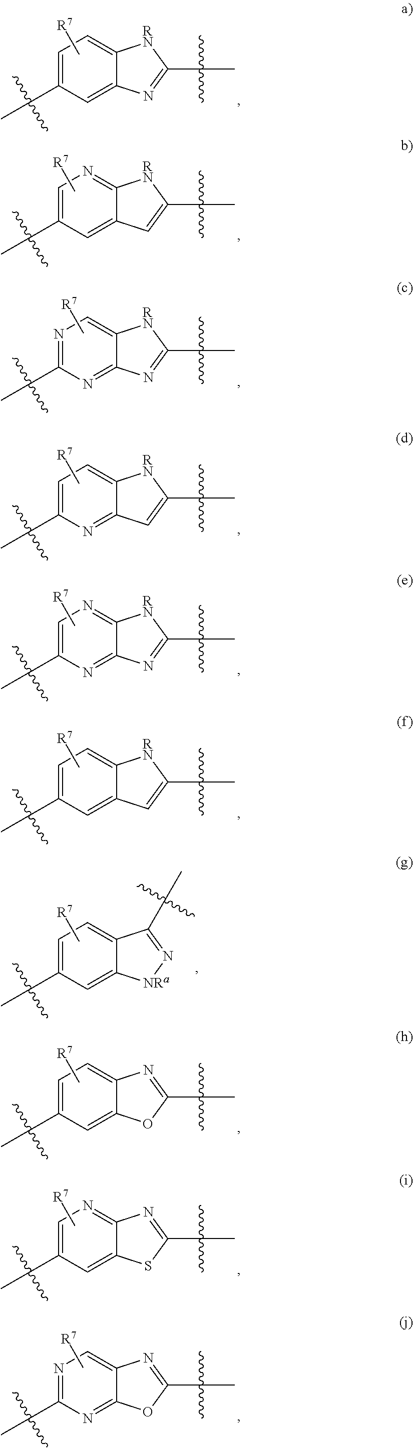

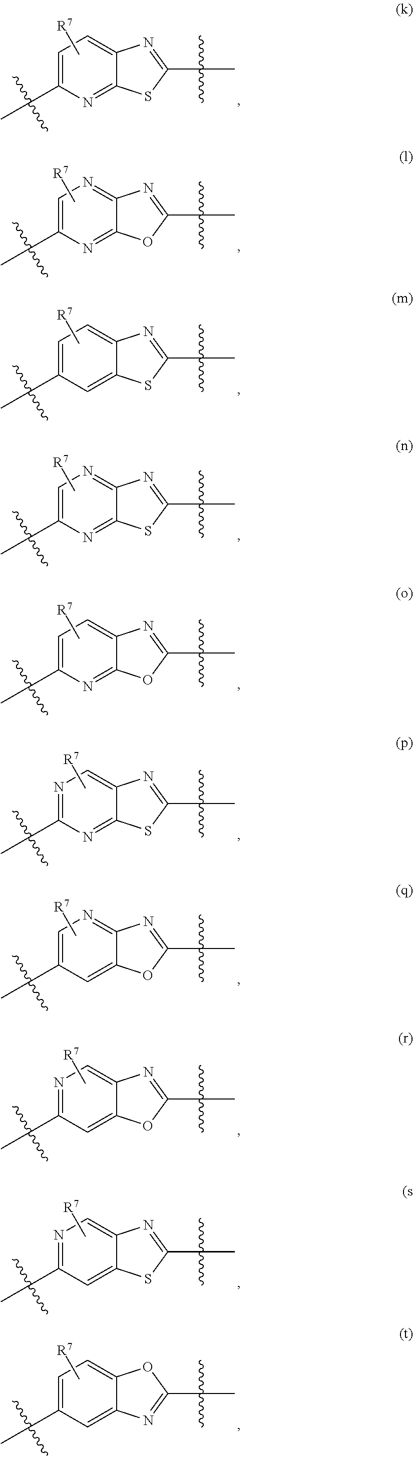

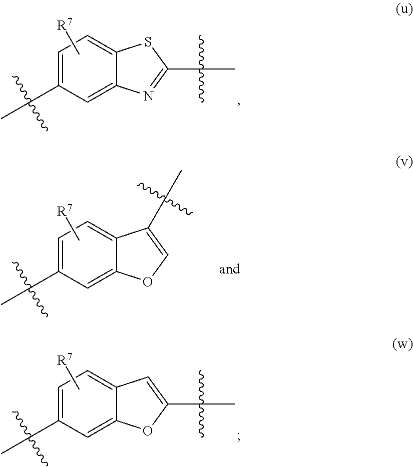

wherein X is selected from O, S, NR and CR³R⁴;

wherein Y is selected from —NR^{b}(CR³R⁴)ₚ—, —NR^{b}C(═O)(CR³R⁴)ₚ—, —NR^{b}C(═O)NR^{b}(CR³R⁴)ₚ—, —NR^{b}C(═O)(CR³R⁴)ₚO—, —NR^{b}C(═O)O(CR³R⁴)ₚ—, —NR^{b}C(═S)(CR³R⁴)ₚ—, —NR^{b}C(═NR^{a})(CR³R⁴)pᵢ>NR^{b}SO₂—(CR³R)ₚ—, —OC(═O)(CR³R⁴)ₚ—, -0(CR³R⁴)ₚ—, —(CR³R⁴)ₚ—S(═O)ᵣ, —(CR³R⁴)_{P}—, —S(═O)ₜNR^{b}(CR³R⁴)ₚ—, —S(═O)ₜ(CR³R⁴)ₚ—, —C(═O)(CR³R⁴)ₚ—, —C(═NR^{a})NH(CR³R⁴)ₚ—, —C(═S)NH(CR³R⁴)_{P}— and —C(═O)NH(CR³R⁴)ₚ—; wherein Y is in either direction;

wherein R^{a} and R^{b} is each independently selected from H, alkyl, heterocyclyl, aryl, arylalkyl, heterocyclylalkyl, cycloalkyl, cycloalkylalkyl, alkenyl, alkynyl, R⁵R⁵N—(C═O)—, and R⁵—(═O)—; wherein each of R^{a} and R^{b} is optionally substituted;

wherein R² is selected from H, alkyl, haloalkyl, aryl, heterocyclyl, arylalkyl, heterocyclylalkyl, cycloalkyl, cycloalkylalkyl, alkenyl, alkynyl and R⁵-carbonyl;

wherein R³ and R⁴ is each independently selected from H, alkyl, aryl, heterocyclyl, arylalkyl, heterocyclylalkyl, haloalkyl, cycloalkyl, cycloalkylalkyl, R⁶ and alkyl substituted with R⁶;

wherein R⁵ is selected from H, alkyl, haloalkyl, arylalkyl, heterocyclylalkyl, cycloalkylalkyl, aryl, heterocyclyl, alkenyl, alkynyl and cycloalkyl;

wherein R^{δ} is selected from cyano, —OR², —SR², halo, —SO₂R², —C(═O)R², —SO₂NR²R⁵, NR⁵C(═O)OR², —NR⁵C(═O)NR⁵R², —NR⁵C(═O)R², —CO₂R², —C(═O)NR²R⁵ and —NR²R⁵;

wherein p is 0, 1, 2, or 3; and

wherein t is 0, 1 or 2; and pharmaceutically acceptable derivatives thereof;

provided R is not 4-chloro-3-(1-methylpynolidin-2-yl)phenyl when Y is NH and A is 2,5-benzoxazolyl and when R¹ is 6,7-dimethoxyquinolinyl; further provided R is not 4-chloro-3-(1-methylpynolidin-2-yl)phenyl when Y is NH and A is 2,5-benzoxazolyl and when R¹ is 6,7-dimethoxyquinazolinyl; further provided R is not phenyl when Y is CH₂ and A is 2,5-benzimidazolyl and when R¹ is 6,7-dimethoxyquinolinyl; further provided Y is not —NH— or —NMe- when X is O, S, CH₂ or NH, and A is benzimidazolyl, benzoxazolyl or benzothiazolyl; and further provided R is not methyl when Y is —(CR³R)ₚ—, when p is 0, and A is 2,5-indolyl.

In one embodiment, Compound GG has the following structure:

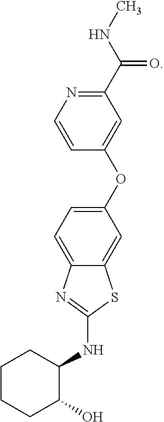 [SMILES: CNC(=O)c1cc(Oc2ccc3nc(N[C@@H]4CCCC[C@H]4O)sc3c2)ccn1]

In one embodiment, Compound GG is 4-((2-(((1R,2R)-2-hydroxycyclohexyl)amino)benzo[d]thiazol-6-yl)oxy)-N-methylpicolinamide.

### Pharmaceutical Compositions and Kits

In another aspect, the present invention provides compositions, e.g., pharmaceutically acceptable compositions, which include an antibody molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules can be administered by a variety of methods known in the art, although for many therapeutic applications, the preferred route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Methods of administering the antibody molecules are known in the art and are described below. Suitable dosages of the molecules used will depend on the age and weight of the subject and the particular drug used. Dosages and therapeutic regimens of the anti-PD-1 antibody or anti-PD-L1 antibody molecule can be determined by a skilled artisan.

In certain embodiments, the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. In some embodiments, the anti-PD-1 antibody molecule is administered by injection (e.g., subcutaneously or intravenously) at a dose of about 50 mg to 500 mg, e.g., 100 mg to 400 mg, 150 mg to 250 mg, or 200 mg to 300 mg, e.g., 200 mg, The dosing schedule can vary from e.g., once a week to once every 2, 3, or 4 weeks. In one embodiment, the anti-PD-1 antibody molecule is administered at a dose from about 10 to 20 mg/kg every other week. In one embodiment is administered at a dose from about 1 mg/kg to 5 mg/kg, e.g., 3 mg/kg, once every three weeks.

In certain embodiments, the anti-PD-L1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, 4, 5 or 6 weeks. In one embodiment, the anti-PD-L1 antibody molecule is administered at a dose from about 1 mg/kg to 5 mg/kg, e.g., 3 mg/kg, once every four weeks. An exemplary, non-limiting range for a therapeutically or prophylactically effective amount of an antibody molecule is 0.1-30 mg/kg, more preferably 1-25 mg/kg. Dosages and therapeutic regimens of the anti-PD-1 antibody molecule or anti-PD-L1 antibody molecule can be determined by a skilled artisan. In certain embodiments, the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week. The antibody molecule can be administered by intravenous infusion at a rate of less than 10 mg/min, preferably less than or equal to 5 mg/min to reach a dose of about 1 to 100 mg/m², preferably about 5 to 50 mg/m², about 7 to 25 mg/m², and more preferably, about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The antibody molecules can be used by themselves or conjugated to a second agent, e.g., a cytotoxic drug, radioisotope, or a protein, e.g., a protein toxin or a viral protein. This method includes: administering the antibody molecule, alone or conjugated to a cytotoxic drug, to a subject requiring such treatment. The antibody molecules can be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g.; via a viral coat protein), or mixtures thereof.

Also within the scope of the invention is a kit comprising a combination therapy described herein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

### Uses of Combination Therapies

The combination therapies disclosed herein have in vitro and in vivo therapeutic and prophylactic utilities. For example, these molecules can be administered to cells in culture, in vitro or ex vivo, or to a subject, e.g., a human subject, to treat, prevent, and/or diagnose a variety of disorders, such as cancers.

Accordingly, in one aspect, the invention provides a method of modifying an immune response in a subject comprising administering to the subject the antibody molecule described herein, such that the immune response in the subject is modified. In one embodiment, the immune response is enhanced, stimulated or up-regulated. In one embodiment, the antibody molecules enhance an immune response in a subject by blockade of a checkpoint inhibitor (e.g., PD-1, PD-L1, LAG-3 or TIM-3).

As used herein, the term “subject” is intended to include human and non-human animals. In one embodiment, the subject is a human subject, e.g., a human patient having a disorder or condition characterized by abnormal immune functioning. The term “non-human animals” includes mammals and non-mammals, such as non-human primates. In one embodiment, the subject is a human. In one embodiment, the subject is a human patient in need of enhancement of an immune response. In one embodiment, the subject is immunocompromised, e.g., the subject is undergoing, or has undergone a chemotherapeutic or radiation therapy. Alternatively, or in combination, the subject is, or is at risk of being, immunocompromised as a result of an infection. The methods and compositions described herein are suitable for treating human patients having a disorder that can be treated by augmenting the T-cell mediated immune response. For example, the methods and compositions described herein can enhance a number of immune activities. In one embodiment, the subject has increased number or activity of tumour-infiltrating T lymphocytes (TILs). In another embodiment, the subject has increased expression or activity of interferon-gamma (IFN-γ). In yet another embodiment, the subject has decreased PD-L1 expression or activity.

### Cancer

Blockade of checkpoint inhibitors, e.g., PD-1, can enhance an immune response to cancerous cells in a subject. The ligand for PD-1, PD-L1, is not expressed in normal human cells, but is abundant in a variety of human cancers (Dong et al. (2002) Nat Med 8:787-9). The interaction between PD-1 and PD-L1 can result in a decrease in tumor infiltrating lymphocytes, a decrease in T-cell receptor mediated proliferation, and/or immune evasion by the cancerous cells (Dong et al. (2003) J Mol Med 81:281-7; Blank et al. (2005) Cancer Immunol. Immunother. 54:307-314; Konishi et al. (2004) Clin. Cancer Res. 10:5094-100).

In one aspect, the invention relates to treatment of a subject in vivo using an anti-PD-1 or anti-PD-L1 antibody molecule such that growth of cancerous tumors is inhibited or reduced. An anti-PD-1 or anti-PD-L1 antibody may be used alone to inhibit the growth of cancerous tumors. Alternatively, an anti-PD-1 or anti-PD-L1 antibody may be used in combination with one or more of: an agent disclosed in Table 1, a standard of care treatment (e.g., for cancers), another antibody or antigen-binding fragment thereof, another immunomodulator (e.g., an activator of a costimulatory molecule or an inhibitor of an inhibitory molecule); a vaccine, e.g., a therapeutic cancer vaccine; or other forms of cellular immunotherapy, as described below.

Accordingly, in one embodiment, the invention provides a method of inhibiting growth of tumor cells in a subject, comprising administering to the subject a therapeutically effective amount of a combination therapy disclosed herein. In one embodiment, the methods are suitable for the treatment of cancer in vivo. When antibodies to PD-1 or PD-L1 are administered in combination with one or more agents, the combination can be administered in either order or simultaneously.

In another aspect, a method of treating a subject, e.g., reducing or ameliorating, a hyperproliferative condition or disorder (e.g., a cancer), e.g., solid tumor, a soft tissue tumor, or a metastatic lesion, in a subject is provided. The method includes administering to the subject one or more anti-PD-1 or PD-L1 antibody molecules described herein, alone or in combination with other agents or therapeutic modalities.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the antibodies molecules disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer (e.g., a NSCLC with squamous and/or non-squamous histology, or a NSCLC adenocarcinoma)). Additionally, refractory or recurrent malignancies can be treated using the antibody molecules described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

Treatment of metastatic cancers, e.g., metastatic cancers that express PD-L1 (Iwai et al. (2005) Int. Immunol. 17:133-144) can be effected using the antibody molecules described herein. In one embodiment, the cancer expresses an elevated level of PD-L1, IFNγ and/or CD8.

Hematological cancer conditions are the types of cancer such as leukemia and malignant lymphoproliferative conditions that affect blood, bone marrow and the lymphatic system. Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

In other embodiments, the cancer is a hematological malignancy or cancer including but is not limited to a leukemia or a lymphoma. For example, the combination therapy can be used to treat cancers and malignancies including, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like.

In one embodiment, the cancer is chosen from a lung cancer (e.g., a non-small cell lung cancer (NSCLC) (e.g., a NSCLC with squamous and/or non-squamous histology)), a melanoma (e.g., an advanced melanoma), a renal cancer (e.g., a renal cell carcinoma, e.g., clear cell renal cell carcinoma), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, a non-Hogdkin's lymphoma, or a leukemia (e.g., a myeloid leukemia).

In another embodiment, the cancer is chosen form a carcinoma (e.g., advanced or metastatic carcinoma), melanoma or a lung carcinoma, e.g., a non-small cell lung carcinoma.

In one embodiment, the cancer is a lung cancer, e.g., a non-small cell lung cancer.

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC or clear cell renal cell carcinoma).

In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In yet other embodiments, the anti-PD-1 or PD-L1 antibody molecule is administered after treatment with an anti-CTLA4 antibody (e.g., ipilimumab) with or without a BRAF inhibitor (e.g., vemurafenib or dabrafenib).

Methods and compositions disclosed herein are useful for treating metastatic lesions associated with the aforementioned cancers.

### Additional Combination Therapies

The combination therapy disclosed herein can be further co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

This section discusses other combinations of immunomodulators with various second therapeutics. Many of the combinations in this section are useful in treating cancer, but other indications are also described. This section focuses on combinations of PD-1 with the agents described in Table 1.

In some embodiments, the immunomodulator, e.g., the inhibitor of an immune checkpoint molecule as described herein, alone or in combination with one or more other immunomodulators, is administered in combination with Compound Q.

In some embodiments, the immunomodulator, e.g., the inhibitor of an immune checkpoint molecule as described herein, alone or in combination with one or more other immunomodulators, is used in combination with an anti-cancer agent that preserves anti-cancer immune cell function. In one embodiment, the immunomodulator is a PD-1 inhibitor, e.g., the anti-PD-1 antibody (e.g., Nivolumab or Pembrolizumab); or a PD-L1 inhibitor, e.g., the anti-PD-L1 antibody (e.g., MSB0010718C). While not wishing to be bound by theory, in some embodiments it is beneficial to administer a PD-1 antibody to a patient that has anti-cancer immune cells available to act against the cancer. The impact of an anti-cancer agent on immune cell function can be measured, e.g., in one or more of a huMLR assay, a T cell proliferation assay, and a B-cell proliferation assay. Exemplary assays are described below. Based on the assay, an IC50 for can be calculated for each test agent. In some embodiments, the anti-cancer agent that is combined with the PD-1 antibody is an anti-cancer agent that has a relatively high IC50 in this assay, e.g., an IC50 of greater than about 0.5, 1, 2, 3, 4, 6, 8, or 10 μM. In some embodiments, the anti-cancer agent has an IC50 in this assay that is higher than its expected level in the patient (e.g., in the patient's bloodstream or in the tumor) when administered at a therapeutic dose to the patient. For example, the IC50 in this assay may be at least 2, 3, 4, 5, 10, 20, 50, or 100-fold higher than the expected level of the drug in the patient. In some embodiments, the drug anti-cancer agent that preserves anti-cancer immune cell function is selected from: Compound D, Compound I, Compound K, Compound L, Compound R, or Compound U, e.g., as described in Table 1, In embodiments, the anti-cancer agent is a compound of a genus encompassing a compound of the previous sentence, as described in Table 1.

Exemplary huMLR assay and B or T cell proliferation assays are provided below.

Human Mixed Lymphocyte Reaction

The Mixed Lymphocyte Reaction (MLR) is a functional assay which measures the proliferative response of lymphocytes from one individual (the responder) to lymphocytes from another individual (the stimulator). To perform an allogeneic MLR, peripheral blood mononuclear cells (PBMC) from three donors were isolated from buffy-coats of unknown HLA type (Kantonspital Blutspendezentrum from Bern and Aarau, Switzerland). The cells were prepared at 2.105 in 0.2 mL of culture medium containing RPMI 1640 GlutaMAX™ with 10% fetal calf serum (FCS), 100 U penicillin/100 μg streptomycin, 50 μM 2-Mercaptoethanol. Individual 2-way reactions were set up by mixing PBMC from two different donors at a 1:1 ratio and co-cultures were done in triplicates in flat-bottomed 96-well tissue culture plates for 6 days at 37° C., 5% CO2, in presence or not of an 8-point concentration range of test compounds. Cells were pulsed with 3H-TdR (1 μCi/0.2 mL) for the last 16 h of culture and incorporated radioactivity was used as a measure of cell proliferation. The concentration that inhibited 50% of the maximal huMLR response (IC50) was calculated for each compound. Cyclosporine was used as a positive control of huMLR inhibition.

Human B Cell Proliferation Assay

PBMC were freshly isolated by Ficoll-Paque density gradient from human blood and subjected to negative B-cell isolation. B cells were resuspended in culture medium (RPMI 1640, HEPES, 10% FCS, 50 μg/mL gentamicine, 50 μM 2-Mercaptoethanol, 1×ITS (Insulin, Transferrin and Sodium Selenite), 1× Non-Essential Amino-Acids) at a concentration of 9.104 per well in a flat-bottom 96-well culture plate. B cell stimulation was performed by human anti-IgM antibody (30 ug/mL) and IL-4 (75 ng/mL) or by CD40 ligand (3 ug/mL) and IL-4 (75 ng/mL) in presence or not of a 7-point concentration range of test compounds. After 72 h of culture at 37° C., 10% CO2, cells were pulsed with 3H-TdR (1 μCi/well) for the last 6 h of culture. B cells were then harvested and the incorporation of thymidine was measured using a scintillation counter. Of each duplicate treatment, the mean was calculated and these data were plotted in XLfit 4 to determine the respective IC50 values.

Human T Cell Proliferation Assay

PBMC were freshly isolated by Ficoll-Paque density gradient from human blood and subjected to negative isolation of T cells. T cells were prepared in culture medium (RPMI 1640, HEPES, 10% FCS, 50 μg/mL gentamicine, 50 μM 2-Mercaptoethanol, lx ITS (Insulin, Transferrin and Sodium Selenite), lx Non-Essential Amino-Acids) at a concentration of 8.104 per well in a flat-bottom 96-well culture plate. T cell stimulation was performed by human anti-CD3 antibody (10 ug/mL) or by human anti-CD3 antibody (5 μg/mL) and anti-CD28 antibody (1 μg/mL) in presence or not of a 7-point concentration range of test compounds. After 72 h of culture at 37° C., 10% CO2, cells were pulsed with 3H-TdR (1 μCi/well) for the last 6 h of culture. Cell proliferation was measured by the incorporation of thymidine allowing IC50 determination for each tested compound.

For example, the combination therapies disclosed herein can also be combined with a standard cancer treatment. For example, PD-1 blockade may be effectively combined with chemotherapeutic regimes. In these instances, it may be possible to reduce the dose of chemotherapeutic reagent administered (Mokyr, M. et al. (1998) Cancer Research 58: 5301-5304). In certain embodiments, the methods and compositions described herein are administered in combination with one or more of other antibody molecules, chemotherapy, other anti-cancer therapy (e.g., targeted anti-cancer therapies, or oncolytic drugs), cytotoxic agents, immune-based therapies (e.g., cytokines), surgical and/or radiation procedures. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation).

Exemplary combinations of with the standard of care for cancer, include at least the following.

In certain embodiments, the combination therapy, is used in combination with a standard of cancer care chemotherapeutic agent including, but not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids that can be used in combination with a combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors that can be used in combination with combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), include, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912); danoprevir (RG7227, CAS 850876-88-9); ixazomib (MLN2238, CAS 1072833-77-2); and (S)—N-[(phenylmethoxy)carbonyl]-L-leucyl-N-(1-formyl-3-methylbutyl)-L-Leucinamide (MG-132, CAS 133407-82-6).

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Further examples of hedgehog inhibitors include, but are not limited to, vismodegib (2-chloro-N-[4-chloro-3-(2-pyridinyl)phenyl]-4-(methylsulfonyl)-benzamide, GDC-0449, described in PCT Publication No. WO 06/028958); 1-(4-Chloro-3-(trifluoromethyl)phenyl)-3-((3-(4-fluorophenyl)-3,4-dihydro-4-oxo-2-quinazolinyl)methyl)-urea (CAS 330796-24-2); N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-2(3H),9′(1′H)-naphth[2,1-a]azulen]-3′-yl]-methanesulfonamide (IPI926, CAS 1037210-93-7); and 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9); and Erismodegib (LDE225). Selected tyrosine kinase inhibitors are chosen from sunitinib, erlotinib, gefitinib; or sorafenib erlotinib hydrochloride (Tarceva®); linifanib (N-[4-(3-amino-1H-indazol-4-yl)phenyl]-N′-(2-fluoro-5-methylphenyl)urea, also known as ABT 869, available from Genentech); sunitinib malate (Sutent®); bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, described in U.S. Pat. No. 6,780,996); dasatinib (Sprycel®); pazopanib (Votrient®); sorafenib (Nexavar®); zactima (ZD6474); and imatinib or imatinib mesylate (Gilvec® and Gleevec®).

In certain embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a Vascular Endothelial Growth Factor (VEGF) receptor inhibitors, including but not limited to, Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary anti-VEGF antibodies include, but are not limited to, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599. In one embodiment, the anti-VEGF antibody is Bevacizumab (BV), also known as rhuMAb VEGF or AVASTIN®. It comprises mutated human IgG1 framework regions and antigen-binding complementarity-determining regions from the murine anti-hVEGF monoclonal antibody A.4.6.1 that blocks binding of human VEGF to its receptors. Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, the contents of these patent applications are expressly incorporated herein by reference. For additional antibodies see U.S. Pat. Nos. 7,060,269, 6,582,959, 6,703,020, 6,054,297, WO98/45332, WO 96/30046, WO94/10202, EP 0666868B1, U.S. Patent Application Publication Nos. 2006009360, 20050186208, 20030206899, 20030190317, 20030203409, and 20050112126; and Popkov et al, Journal of Immunological Methods 288: 149-164 (2004). Other antibodies include those that bind to a functional epitope on human VEGF comprising of residues F17, M18, D19, Y21, Y25, Q89, 191, K1 01, El 03, and C104 or, alternatively, comprising residues F17, Y21, Q22, Y25, D63, 183 and Q89.

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a PI3K inhibitor. In one embodiment, the PI3K inhibitor is an inhibitor of delta and gamma isoforms of PI3K. Exemplary PI3K inhibitors that can be used in combination are described in, e.g., WO 2010/036380, WO 2010/006086, WO 09/114870, WO 05/113556, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). Further examples of PI3K inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941, described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ235 or NVP-BEZ 235, described in PCT Publication No. WO 06/122806); 4-(trifluoromethyl)-5-(2,6-dimorpholinopyrimidin-4-yl)pyridin-2-amine (also known as BKM120 or NVP-BKM120, described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); 2-Amino-8-ethyl-4-methyl-6-(1H-pyrazol-5-yl)pyrido[2,3-d]pyrimidin-7(8H)-one (SAR 245409 or XL 765); 1,3-Dihydro-8-(6-methoxy-3-pyridinyl)-3-methyl-1-[4-(1-piperazinyl)-3-(trifluoromethyl)phenyl]-2H-imidazo[4,5-c]quinolin-2-one, (2Z)-2-butenedioate (1:1) (BGT 226); 5-Fluoro-3-phenyl-2-[(1S)-1-(9H-purin-6-ylamino)ethyl]-4(3H)-quinazolinone (CAL101); 2-Amino-N-[3-[N-[3-[(2-chloro-5-methoxyphenyl)amino]quinoxalin-2-yl]sulfamoyl]phenyl]-2-methylpropanamide (SAR 245408 or XL 147); and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (BYL719).

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a mTOR inhibitor, e.g., one or more mTOR inhibitors chosen from one or more of rapamycin, temsirolimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), (1r,4r)-4-(4-amino-5-(7-methoxy-1H-indol-2-yl)imidazo[1,5-f][1,2,4]triazin-7-yl)cyclohexanecarboxylic acid (OSI-027); and XL765.

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a BRAF inhibitor, e.g., GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7); tuvizanib (AV951, CAS 475108-18-0); vemurafenib (Zelboraf®, PLX-4032, CAS 918504-65-1); encorafenib (also known as LGX818); 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1H-pyrrolo[2,3-b]pyridine-3-carbonyl)-2,4-difluorophenyl)propane-1-sulfonamide (also known as PLX4720).

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a MEK inhibitor. In some embodiments, the combination of the anti-PD-1 antibody and the MEK inhibitor is used to treat a cancer (e.g., a cancer described herein). In some embodiments, the cancer treated with the combination is chosen from a melanoma, a colorectal cancer, a non-small cell lung cancer, an ovarian cancer, a breast cancer, a prostate cancer, a pancreatic cancer, a hematological malignancy or a renal cell carcinoma. In certain embodiments, the cancer includes a BRAF mutation (e.g., a BRAF V600E mutation), a BRAF wildtype, a KRAS wildtype or an activating KRAS mutation. The cancer may be at an early, intermediate or late stage. Any MEK inhibitor can be used in combination including, but not limited to, selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO2003077914); ARRY-142886 trametinib dimethyl sulfoxide (GSK-1120212, CAS 1204531-25-80); G02442104 (also known as GSK1120212), RDEA436; N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766, described in PCT Publication No. WO2007014011); RDEA119/BAY 869766, AS703026; G00039805 (also known as AZD-6244 or selumetinib), BIX 02188; BIX 02189; 2-[(2-Chloro-4-iodophenyl)amino]-N-(cyclopropylmethoxy)-3,4-difluoro-benzamide (also known as CI-1040 or PD184352, described in PCT Publication No. WO2000035436); CI-1040 (PD-184352), N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); PD03259012′-amino-3′-methoxyflavone (also known as PD98059 available from Biaffin GmbH & Co., KG, Germany); PD98059, 2,3-bis[amino[(2-aminophenyl)thio]methylene]-butanedinitrile (also known as U0126 and described in U.S. Pat. No. 2,779,780); U0126, XL-518 (also known as GDC-0973, Cas No. 1029872-29-4, available from ACC Corp.); GDC-0973 (Methanone, [3,4-difluoro-2-[(2-fluoro-4-iodophenyl)amino]phenyl][3-hydroxy-3-(25)-2-piperidinyl-1-azetidinyl]-), G-38963; and G02443714 (also known as AS703206), or a pharmaceutically acceptable salt or solvate thereof. Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, benimetinib (6-(4-bromo-2-fluorophenylamino)-7-fluoro-3-methyl-3H-benzoimidazole-5-carboxylic acid (2-hydroxyethyoxy)-amide, also known as MEK162, CAS 1073666-70-2, described in PCT Publication No. WO2003077914); 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, the combination therapy disclosed herein (e.g., an anti-PD-1 or PD-L1 antibody molecule, alone or in combination with another immunomodulator (e.g., an anti-LAG-3, or anti-TIM-3 antibody molecule) and a compound of Table 1), in combination with a JAK2 inhibitor, e.g., CEP-701, INCB18424, CP-690550 (tasocitinib). Exemplary JAK inhibitors include, but are not limited to, ruxolitinib (Jakafi®); tofacitinib (CP690550); axitinib (AG013736, CAS 319460-85-0); 5-Chloro-N2-[(1S)-1-(5-fluoro-2-pyrimidinyl)ethyl]-N4-(5-methyl-1H-pyrazol-3-yl)-12,4-pyrimidinediamine (AZD1480, CAS 935666-88-9); (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6); momelotinib (CYT 387); baricitinib (INCB-028050 or LY-3009104); pacritinib (SB1518); (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317); gandotinib (LY 2784544); and N,N-cicyclopropyl-4-[(1,5-dimethyl-1H-pyrazol-3-yl)amino]-6-ethyl-1,6-dihydro-1-methyl-imidazo[4,5-d]pyrrolo[2,3-b]pyridine-7-carboxamide (BMS 911543).

In some embodiments, the combination therapies disclosed herein include paclitaxel or a paclitaxel agent, e.g., TAXOL®, protein-bound paclitaxel (e.g., ABRAXANE®). Exemplary paclitaxel agents include, but are not limited to, nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

In certain embodiments, the anti-PD-1 or PD-L1 antibody molecule, alone or in combination with another immunomodulator (e.g., an anti-LAG-3 or anti-TIM-3 antibody molecule), is administered in combination with an antibody against a Killer-cell Immunoglobulin-like Receptors (also referred to herein as an “anti-KIR antibody”). In certain embodiments, the combination of anti-PD-1 antibody molecule and anti-KIR antibody described herein is used to treat a cancer, e.g., a cancer as described herein (e.g., a solid tumor, e.g., an advanced solid tumor).

In one embodiment, the anti-PD-1 or PD-L1 antibody molecule, alone or in combination with another immunomodulator (e.g., an anti-LAG-3 or anti-TIM-3 antibody molecule), is administered in combination with a cellular immunotherapy (e.g., Provenge (e.g., Sipuleucel)), and optionally in combination with cyclophosphamide. In certain embodiments, the combination of anti-PD-1 antibody molecule, Provenge and/or cyclophosphamide is used to treat a cancer, e.g., a cancer as described herein (e.g., a prostate cancer, e.g., an advanced prostate cancer).

In another embodiment, the anti-PD-1 or PD-L1 antibody molecule, alone or in combination with another immunomodulator (e.g., an anti-LAG-3 or anti-TIM-3 antibody molecule), is administered in combination with a vaccine, e.g., a dendritic cell renal carcinoma

(DC-RCC) vaccine. In certain embodiments, the combination of anti-PD-1 antibody molecule and the DC-RCC vaccine is used to treat a cancer, e.g., a cancer as described herein (e.g., a renal carcinoma, e.g., metastatic renal cell carcinoma (RCC) or clear cell renal cell carcinoma (CCRCC)).

In yet another embodiment, the anti-PD-1 or PD-L1 antibody molecule, alone or in combination with another immunomodulator (e.g., an anti-LAG-3 or anti-TIM-3 antibody molecule), is administered in combination with chemotherapy, and/or immunotherapy. For example, the anti-PD-1 or PD-L1 antibody molecule can be used to treat a myeloma, alone or in combination with one or more of: chemotherapy or other anti-cancer agents (e.g., thalidomide analogs, e.g., lenalidomide), an anti-TIM3 antibody, tumor antigen-pulsed dendritic cells, fusions (e.g., electrofusions) of tumor cells and dendritic cells, or vaccination with immunoglobulin idiotype produced by malignant plasma cells. In one embodiment, the anti-PD-1 or PD-L1 antibody molecule is used in combination with an anti-TIM-3 antibody to treat a myeloma, e.g., a multiple myeloma.

In one embodiment, the anti-PD-1 or PD-L1 antibody molecule, alone or in combination with another immunomodulator (e.g., an anti-LAG-3 or anti-TIM-3 antibody molecule), is used in combination with chemotherapy to treat a lung cancer, e.g., non-small cell lung cancer. In one embodiment, the anti-PD-1 or PD-L1 antibody molecule is used with platinum doublet therapy to treat lung cancer.

In yet another embodiment, the anti-PD-1 or PD-L1 antibody molecule, alone or in combination with another immunomodulator (e.g., an anti-LAG-3 or anti-TIM-3 antibody molecule), is used to treat a renal cancer, e.g., renal cell carcinoma (RCC) (e.g., clear cell renal cell carcinoma (CCRCC) or metastatic RCC. The anti-PD-1 or PD-L1 antibody molecule can be administered in combination with one or more of: an immune-based strategy (e.g., interleukin-2 or interferon-α), a targeted agent (e.g., a VEGF inhibitor such as a monoclonal antibody to VEGF); a VEGF tyrosine kinase inhibitor such as sunitinib, sorafenib, axitinib and pazopanib; an RNAi inhibitor), or an inhibitor of a downstream mediator of VEGF signaling, e.g., an inhibitor of the mammalian target of rapamycin (mTOR), e.g., everolimus and temsirolimus.

An example of suitable therapeutics for use in combination for treatment of pancreatic cancer includes, but is not limited to, a chemotherapeutic agent, e.g., paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., RO5126766); Notch signaling inhibitor (e.g., MK0752); monoclonal antibody-antibody fusion protein (e.g., L19IL2); curcumin; HSP90 inhibitor (e.g., tanespimycin, STA-9090); rIL-2; denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., R04929097); ribonucleotide reductase inhibitor (e.g., 3-AP); immunotoxin (e.g., HuC242-DM4); PARP inhibitor (e.g., KU-0059436, veliparib); CTLA-4 inhbitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof. In certain embodiments, a combination of paclitaxel or a paclitaxel agent, and gemcitabine can be used with the anti-PD-1 antibody molecules described herein.

An example of suitable therapeutics for use in combination for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., tanespimycin, STA-9090); mTOR inhibitor (e.g., everolimus); Ep-CAM-/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

An example of suitable therapeutics for use in combination for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, R05083945), MET inhibitor (e.g., PF-02341066, ARQ 197), PI3K kinase inhibitor (e.g., XL147, GDC-0941), Raf/MEK dual kinase inhibitor (e.g., R05126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fusl, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination for treatment of ovarian cancer includes, but is not limited to, a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agnet (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy; and combinations thereof.

An example of suitable therapeutics for use in combination to treat a myeloma, alone or in combination with one or more of: chemotherapy or other anti-cancer agents (e.g., thalidomide analogs, e.g., lenalidomide), HSCT (Cook, R. (2008) J Manag Care Pharm. 14(7 Suppl):19-25), an anti-TIM3 antibody (Hallett, W H D et al. (2011) J of American Society for Blood and Marrow Transplantation 17(8):1133-145), tumor antigen-pulsed dendritic cells, fusions (e.g., electrofusions) of tumor cells and dendritic cells, or vaccination with immunoglobulin idiotype produced by malignant plasma cells (reviewed in Yi, Q. (2009) Cancer J. 15(6):502-10).

An example of suitable therapeutics for use in combination to treat a renal cancer, e.g., renal cell carcinoma (RCC) or metastatic RCC. The anti-PD-1 antibody molecule can be administered in combination with one or more of: an immune-based strategy (e.g., interleukin-2 or interferon-α), a targeted agent (e.g., a VEGF inhibitor such as a monoclonal antibody to VEGF, e.g., bevacizumab (Rini, B. I. et al. (2010) J. Clin. Oncol. 28(13):2137-2143)); a VEGF tyrosine kinase inhibitor such as sunitinib, sorafenib, axitinib and pazopanib (reviewed in Pal. S. K. et al. (2014) Clin. Advances in Hematology & Oncology 12(2):90-99)); an RNAi inhibitor), or an inhibitor of a downstream mediator of VEGF signaling, e.g., an inhibitor of the mammalian target of rapamycin (mTOR), e.g., everolimus and temsirolimus (Hudes, G. et al. (2007) N. Engl. J. Med. 356(22):2271-2281, Motzer, R. J. et al. (2008) Lancet 372: 449-456).

An example of suitable therapeutics for use in combination for treatment of chronic myelogenous leukemia (AML) according to the invention includes, but is not limited to, a chemotherapeutic (e.g., cytarabine, hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib, RGB-286638)), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., R05045337), Aurora B kinase inhibitor (e.g., TAK-901), radioimmunotherapy (e.g., actinium-225-labeled anti-CD33 antibody HuM195), Hedgehog inhibitor (e.g., PF-04449913), STAT3 inhibitor (e.g., OPB-31121), KB004, cancer vaccine (e.g., AG858), bone marrow transplantation, stem cell transplantation, radiation therapy, and combinations thereof.

An example of suitable therapeutics for use in combination for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD-20 targeting agent (e.g., rituximab, ofatumumab, R05072759, LFB-R603), CD52 targeting agent (e.g., alemtuzumab), prednisolone, darbepoetin alfa, lenalidomide, Bcl-2 inhibitor (e.g., ABT-263), immunotherapy (e.g., allogeneic CD4+ memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., R05045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., R05045337), immunotoxin (e.g., CAT-8015, DT2219ARL), HDAC inhibitor (e.g., JNJ-26481585), JVRS-100, paclitaxel or a paclitaxel agent, STAT3 inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT3881L3 fusion protein, HDAC inhibitor (e.g., vorinostat, LBH589), plerixafor, mTOR inhibitor (e.g., everolimus), SRC inhibitor (e.g., dasatinib), HSP90 inhbitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., R05045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of suitable therapeutics for use in combination for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3 inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of suitable therapeutics for use in combination for treatment of prostate cancer includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

The combination therapies can be administered in combination with one or more of the existing modalities for treating cancers, including, but not limited to: surgery; radiation therapy (e.g., external-beam therapy which involves three dimensional, conformal radiation therapy where the field of radiation is designed, local radiation (e.g., radition directed to a preselected target or organ), or focused radiation). Focused radiation can be selected from the group consisting of stereotactic radiosurgery, fractionated stereotactic radiosurgery, and intensity-modulated radiation therapy. The focused radiation can have a radiation source selected from the group consisting of a particle beam (proton), cobalt-60 (photon), and a linear accelerator (x-ray), e.g., as described in WO 2012/177624.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g. At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

### Nucleic Acids

The invention also features nucleic acids comprising nucleotide sequences that encode heavy and light chain variable regions and CDRs or hypervariable loops of the antibody molecules, as described herein. The nucleic acid can comprise a nucleotide sequence as set forth herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

### Vectors

Further provided herein are vectors comprising nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### EXAMPLES

### Example 1: Patient Selection Based on PD-L1/CD8/IFN-γ Status

For each of several types of cancer, samples from multiple patients were tested for PD-L1/CD8/IFN-γ status. Each sample was classified as: triple-negative for PD-L1/CD8/IFN-γ, single or double positive for these markers, or triple-positive for these markers.

FIG. 1 shows that in this experiment, within a population of patients, the following types of cancer are frequently triple-positive for PD-L1/CD8/IFN-γ: Lung cancer (squamous), lung cancer (adenocarcinoma), head and neck cancer, cervical cancer (squamous), stomach cancer, thyroid cancer, melanoma, and nasopharyngeal cancer. Patients having these types of cancer are good candidates for therapy with anti-PD-1 antibodies and combination therapies as described herein. The likelihood of successful treatment can be further boosted by determining which patients are triple-positive for PD-L1/CD8/IFN-γ, and treating the triple-positive patients with anti-PD-1 antibodies (or anti-PD-L1 antibodies) and combination therapies as described herein.

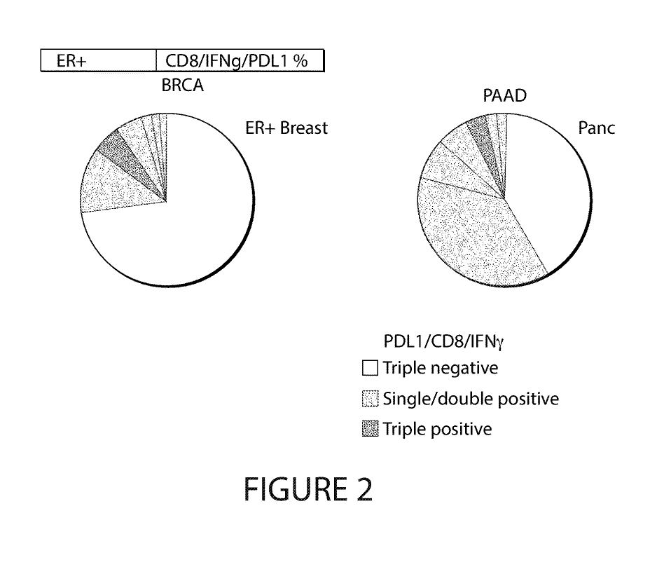

FIG. 2 shows that within a population of patients, the following types of cancer are rarely triple positive for PD-L1/CD8/IFN-γ: ER+ breast cancer and pancreatic cancer. Notably, even in cancers that are generally not positive for PD-L1/CD8/IFN-γ, one can increase the likelihood of successful treatment by determining which patients are triple-positive for PD-L1/CD8/IFN-γ, and treating the triple-positive patients with anti-PD-1 antibodies (or anti-PD-L1 antibodies) and combination therapies as described herein.

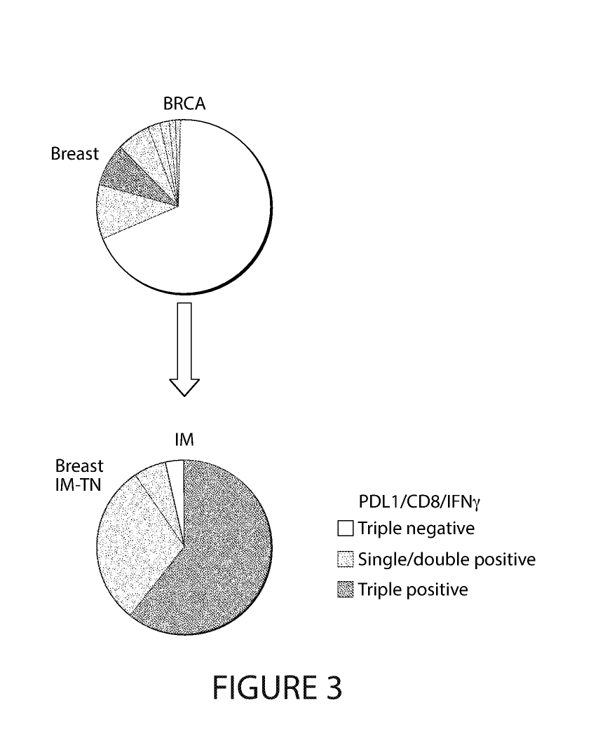

FIG. 3 shows the proportion of breast cancer patients that are triple positive for PD-L1/CD8/IFN-γ. Considering breast cancer in general, the proportion of triple-positives is somewhat low. However, when one focuses only on IM-TN breast cancer, it can be seen that a much larger percentage of patients is triple positive for PD-L1/CD8/IFN-γ. IM-TN breast cancer is particularly difficult to treat with conventional therapies. The discovery that IM-TN breast cancer is often triple-positive for PD-L1/CD8/IFN-γ opens up new avenues of therapy for this cancer with anti-PD-1 antibodies (or anti-PD-L1 antibodies) and combination therapies as described herein.

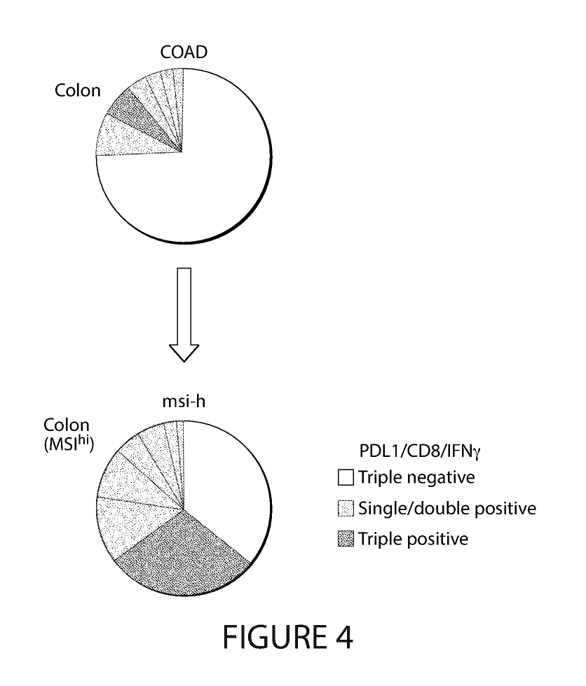

FIG. 4 shows the proportion of colon cancer patients that are triple positive for PDL1/CD8/IFN-γ. Considering colon cancer in general, the proportion of triple-positive is somewhat low. However, when one focuses only on MSI-high (high microsatellite instability) breast cancer, it can be seen that a much larger percentage of patients is triple positive for PD-L1/CD8/IFN-γ. MSI levels can be assayed using, e.g., commercially available PCR-based methods.

Gastric cancer samples were tested for levels of PD-L1/CD8/IFN-γ (data not shown). It was found that in MSI-high or EBV+ gastric cancers, about 49% were positive for PD-L1, and a high proportion of the PD-L1-positive cells were triple positive for PD-L1/CD8/IFN-γ. It was also found that a proportion of PD-L1-positive cells and PD-L1/CD8/IFN-γ positive cells were also positive for PIK3CA. This finding suggests that these cancers may be treated with an anti-PD-1 antibody (or an anti-PD-L1 antibody), optionally in combination with a PIK3 therapeutic.

MSI-high CRC samples were tested for a combination of markers (data not shown). It was found that in MSI-high CRC samples, a high proportion of the PD-L1/CD8/IFN-γ samples are also positive for LAG-3, PD-1 (also called PDCD1), RNF43, and BRAF. This finding suggests that these cancers may be treated with a PD-1 antibody, optionally in combination with a therapeutic that targets one or more of LAG-3, PDCD1, RNF43, and BRAF.

Squamous cell lung cancers were tested for a combination of markers (data not shown). It was found that in squamous cell lung cancer samples, a high proportion of the PD-L1/CD8/IFN-γ samples are also positive for LAG-3. This finding suggests that these cancers may be treated with an anti-PD-1 antibody (or an anti-PD-L1 antibody), optionally in combination with a therapeutic that targets LAG-3, e.g., a LAG-3 antibody.

Papillary thyroid cancers were tested for a combination of markers including the BRAF V600E mutation (data not shown). It was found that a high proportion of thyroid cancer samples that are positive for PD-L1 are also positive for BRAF V600E. This finding suggests that these cancers may be treated with a PD-1 antibody, optionally in combination with a therapeutic that targets BRAF.

### Example 2: Patient Selection Based on PD-L1 Status

To enable broad examination of cancer indications for PD-1/PD-L1 based therapies, we evaluated PD-L1 expression at both the protein and mRNA level in human cancers including both lung and hepatic tumors.

PD-L1 protein expression was evaluated in a set of formalin-fixed paraffin-embedded non-small cell lung (NSCLC) adenocarcinoma (ACA), NSCLC squamous cell carcinoma (SCC), and hepatocellular carcinoma (HCC) tumors by immunohistochemistry (IHC). PD-L1 expression was scored semi-quantitatively by a manual histo-score (H-score) methodology based on staining intensity and percentage of positive tumor cells. In our IHC analysis, PD-L1 positivity (PD-L1+) was defined as an H-score ≧20. In parallel, PD-L1 mRNA expression data was examined from The Cancer Genome Atlas (TCGA) in these same indications (503 NSCLC ACA, 489 NSCLC SCC, and 191 HCC) and analyzed by comparing the expression in matched normal tissues from TCGA.

With RNAseq analysis, data was calculated as log 2 (RPKM+0.1) after RSEM normalization, utilizing OmicSoft RNASeq pipelines across TCGA tumor indications. The expression of PD-L1 is elevated in NSCLC ACA and SCC, relative to that in HCC. By overlaying the distributions and comparing the expression levels across all indications in TCGA, we ranked overexpression profiles for PD-L1 and found the TCGA HCC cohort to have much reduced PD-L1 mRNA levels, with a median level of −0.8 compared to 1.3 for ACA and 1.5 for SCC, which amounts to more than a 2-fold change of median level expression. With RNAseq, our analysis defines 50% of NSCLC adenocarcinoma, 54% of NSCLC squamous cell carcinoma, and 6% of HCC as high expressers for PD-L1.

Tumor cell PD-L1 protein expression was measured in 45 lung adenocarcinoma (ACA) samples, 47 lung squamous cell carcinoma (SCC) samples, and 36 hepatocellular carcinoma (HCC) samples. 16/45 (35.6%) lung ACA, 21/47 (44.7%) lung SCC were PD-L1 positive. In contrast, PD-L1 positivity was seen in only 2/36 (5.6%) HCC samples.

In summary, with IHC and RNAseq analysis in large and independent human NSCLC and HCC sample sets, PD-L1 expression was found to be more enriched in NSCLC than in HCC. Within NSCLC, there are comparable findings between adenocarcinoma and squamous cell carcinomas. Importantly, amongst the large number of samples (128 for IHC and 1183 for RNAseq) in the 3 indications, very good concordance is observed between protein- and mRNA-based analyses. Our finding thus establishes the basis for large scale mRNA-based data mining in TCGA for indications and patient segments that may be enriched for responses to PD1/PD-L1-based immune therapies.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A combination comprising an immunomodulator and a second therapeutic agent for use in treating a cancer in a subject, wherein:
(i) the immunomodulator is an inhibitor of an immune checkpoint molecule or an activator of a costimulatory molecule, or a combination thereof,
wherein the inhibitor of an immune checkpoint molecule is chosen from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, and
wherein the activator of the costimulatory molecule is chosen from an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand; and
(ii) the second therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor, as provided in Table 1.

2. A combination comprising an immunomodulator and a second therapeutic agent for use in treating a cancer in a subject, wherein:
(i) the immunomodulator is an inhibitor of an immune checkpoint molecule or an activator of a costimulatory molecule, or a combination thereof,
wherein the inhibitor of an immune checkpoint molecule is chosen from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, and
wherein the activator of the costimulatory molecule is chosen from an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand; and
(ii) the second therapeutic agent is chosen from one or more of:
1) 3-(1H-indol-3-yl)-4-[2-(4-methyl-1-piperazinyl)-4-quinazolinyl]-1H-pyrrole-2,5-dione;
2) 5-(2,4-dihydroxy-5-isopropylphenyl)-N-ethyl-4-(4-(morpholinomethyl)phenyl)isoxazole-3-carboxamide;
3) 2-methyl-3-(5-(4-methyl-3-oxo-9-(quinolin-3-yl)-2,3-dihydro-1H-imidazo[4,5-c]quinolin-1-yl)phenyl)propanenitrile;
4) Compound D;
5) 4-[3,5-bis(2-hydroxyphenyl)-1H-1,2,4-triazol-1-yl]-benzoic acid;
6) 4,4′-(1H-1,2,4-triazol-1-ylmethylene)bis-benzonitrile;
7) (4S,5R)-3-(2′-amino-2-morpholino-4′-(trifluoromethyl)-[4,5′-bipyrimidin]-6-yl)-4-(hydroxymethyl)-5-methyloxazolidin-2-one;
8) (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one;
9) 4-[(4-methyl-1-piperazinyl)methyl]-N-[4-methyl-3-[[4-(3-pyridinyl)-2-pyrimidinyl]amino]phenyl]-methanesulfonate-benzamide;
10) 4-[(R)-6,7-dihydro-5H-pyrrolo[1,2-c]imidazol-5-yl]-3-fluorobenzonitrile;
11) N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate;
12) (R)-2-(5-(4-(6-benzyl-4,5-dimethylpyridazin-3-yl)-2-methylpiperazin-1-yl)pyrazin-2-yl)propan-2-ol;
13) Compound M;
14) 2-(2′,3-dimethyl-[2,4′-bipyridin]-5-yl)-N-(5-(pyrazin-2-yl)pyridin-2-yl)acetamide;
15) 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide;
16) Compound P;
17) Compound Q;
18) N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide;
19) 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine;
20) cyclo((4R)-4-(2-Aminoethylcarbamoyloxy)-L-prolyl-L-phenylglycyl-D-tryptophyl-L-lysyl-4-O-benzyl-L-tyrosyl-L-phenylalanyl-);
21) 1-amino-5-fluoro-3-[6-(4-methyl-1-piperazinyl)-1H-benzimidazol-2-yl]-2(1H)-quinolinone;
22) 8-(6-Methoxy-pyridin-3-yl)-3-methyl-1-(4-piperazin-1-yl-3-trifluoromethyl-phenyl)-1,3-dihydro-imidazo[4,5-c]quinolin-2-one;
23) N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine;
24) 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide;
25) 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine;
26) 5-chloro-N2-(4-(1-ethylpiperidin-4-yl)-2-fluoro-5-methylphenyl)-N⁴-(5-methyl-1H-pyrazol-3-yl)pyrimidine-2,4-diamine;
27) 6-[(2S,4R,6E)-4-Methyl-2-(methylamino)-3-oxo-6-octenoic acid]cyclosporin D, Amdray, PSC833, [3′-Desoxy-3′-oxo-MeBmt]1-[Val]2-cyclosporin;
28) N-(4-Chlorophenyl)-4-(4-pyridinylmethyl)-1-phthalazinamine succinate;
29) Compound CC;
30) (R)—N-(4-(chlorodifluoromethoxy)phenyl)-6-(3-hydroxypyrrolidin-1-yl)-5-(1H-pyrazol-5-yl)nicotinamide;
31) Compound EE;
32) Compound FF;
33) 4-((2-(((1R,2R)-2-hydroxycyclohexyl)amino)benzo[d]thiazol-6-yl)oxy)-N-methylpicolinamide.

3. A method of treating a cancer in a subject, comprising administering to the subject an immunomodulator and a second therapeutic agent, wherein:
(i) the immunomodulator is chosen from one or more of: an activator of a costimulatory molecule or an inhibitor of an immune checkpoint molecule, or a combination thereof,
wherein the inhibitor of an immune checkpoint molecule is chosen from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, and
wherein the activator of the costimulatory molecule is chosen from an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, and
(ii) the second therapeutic agent is chosen from one or more of the agents: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor, as provided in Table 1,
thereby treating the cancer in the subject.

4. A method of treating a cancer in a subject, comprising administering to the subject an immunomodulator and a second therapeutic agent, wherein:
(i) the immunomodulator is an inhibitor of an immune checkpoint molecule or an activator of a costimulatory molecule, or a combination thereof,
wherein the inhibitor of an immune checkpoint molecule is chosen from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, and
wherein the activator of the costimulatory molecule is chosen from an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand; and
(ii) the second therapeutic agent is chosen from one or more of:
1) 3-(1H-indol-3-yl)-4-[2-(4-methyl-1-piperazinyl)-4-quinazolinyl]-1H-pyrrole-2,5-dione;
2) 5-(2,4-dihydroxy-5-isopropylphenyl)-N-ethyl-4-(4-(morpholinomethyl)phenyl)isoxazole-3-carboxamide;
3) 2-methyl-2-(4-(3-methyl-2-oxo-8-(quinolin-3-yl)-2,3-dihydro-1H-imidazo[4,5-c]quinolin-1-yl)phenyl)propanenitrile;
4) Compound D;
5) 4-[3,5-bis(2-hydroxyphenyl)-1H-1,2,4-triazol-1-yl]-benzoic acid;
6) 4,4′-(1H-1,2,4-triazol-1-ylmethylene)bis-benzonitrile;
7) (4S,5R)-3-(2′-amino-2-morpholino-4′-(trifluoromethyl)-[4,5′-bipyrimidin]-6-yl)-4-(hydroxymethyl)-5-methyloxazolidin-2-one;
8) (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one;
9) 4-[(4-methyl-1-piperazinyl)methyl]-N-[4-methyl-3-[[4-(3-pyridinyl)-2-pyrimidinyl]amino]phenyl]-methanesulfonate-benzamide;
10) 4-[(5)-6,7-dihydro-5H-pyrrolo[1,2-c]imidazol-5-yl]-3-fluorobenzonitrile;
11) N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate;
12) (R)-2-(5-(4-(6-benzyl-4,5-dimethylpyridazin-3-yl)-2-methylpiperazin-1-yl)pyrazin-2-yl)propan-2-ol;
13) Compound M;
14) 2-(2′,3-dimethyl-[2,4′-bipyridin]-5-yl)-N-(5-(pyrazin-2-yl)pyridin-2-yl)acetamide;
15) 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide;
16) Compound P;
17) Compound Q;
18) N—[(R9,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide;
19) 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine;
20) cyclo((4R)-4-(2-Aminoethylcarbamoyloxy)-L-prolyl-L-phenylglycyl-D-tryptophyl-L-lysyl-4-O-benzyl-L-tyrosyl-L-phenylalanyl-);
21) 1-amino-5-fluoro-3-[6-(4-methyl-1-piperazinyl)-1H-benzimidazol-2-yl]-2(1H)-quinolinone;
22) 8-(6-Methoxy-pyridin-3-yl)-3-methyl-1-(4-piperazin-1-yl-3-trifluoromethyl-phenyl)-1,3-dihydro-imidazo[4,5-c]quinolin-2-one;
23) N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine;
24) 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide;
25) 5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine;
26) 5-chloro-N2-(4-(1-ethylpiperidin-4-yl)-2-fluoro-5-methylphenyl)-N⁴-(5-methyl-1H-pyrazol-3-yl)pyrimidine-2,4-diamine;
27) 6-[(2S,4R,6E)-4-Methyl-2-(methylamino)-3-oxo-6-octenoic acid]cyclosporin D, Amdray, PSC833, [3′-Desoxy-3′-oxo-MeBmt]1-[Val]2-cyclosporin;
28) N-(4-Chlorophenyl)-4-(4-pyridinylmethyl)-1-phthalazinamine succinate;
29) Compound CC;
30) (R)—N-(4-(chlorodifluoromethoxy)phenyl)-6-(3-hydroxypyrrolidin-1-yl)-5-(1H-pyrazol-5-yl)nicotinamide;
31) Compound EE;
32) Compound FF;
33) 4-((2-(((1R,2R)-2-hydroxycyclohexyl)amino)benzo[d]thiazol-6-yl)oxy)-N-methylpicolinamide,
thereby treating the cancer in the subject.

5. A method of reducing growth, survival, or viability, or all, of a cancer cell, comprising contacting the cell with an immunomodulator and a second therapeutic agent, wherein:
(i) the immunomodulator is chosen from one or more of: an activator of a costimulatory molecule or an inhibitor of an immune checkpoint molecule, or a combination thereof,
wherein the inhibitor of an immune checkpoint molecule is chosen from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, and
wherein the activator of the costimulatory molecule is chosen from an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, and
(ii) the second therapeutic agent is chosen from one or more of the agents 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or 17alpha-Hydroxylase/C17-20 Lyase); 5) an iron chelating agent; 6) an aromatase inhibitor; 7) an inhibitor of p53, e.g., an inhibitor of a p53/Mdm2 interaction; 8) an apoptosis inducer; 9) a transduction modulator and/or angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) an inhibitor of fibroblast growth factor receptor 2 (FGFR2) and/or fibroblast growth factor receptor 4 (FGFR4); 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) an isocitrate dehydrogenase (IDH) inhibitor; 25) a BCL-ABL inhibitor; 26) a cRAF inhibitor; 27) an ERK1/2 ATP inhibitor; or 28) a tyrosine kinase (e.g., CSF-1R tyrosine kinase) inhibitor, as provided in Table 1,
thereby reducing the growth, survival, or viability of the cancer cell.

6. The combination for use of claim 1 or 2, or the method of any of claims 3-5, wherein the inhibitor of the immune checkpoint molecule is chosen from an inhibitor of PD-1, PD-L1, LAG-3, TIM-3, CEACAM-1, CEACAM-3, CEACAM-5, or CTLA4, or any combination thereof.

7. The combination for use of any of claim 1-2 or 6, or the method of any of claims 3-6, wherein the agonist of the costimulatory molecule is chosen from an agonist of one or more of OX40, ICOS (CD278), 4-1BB (CD137), GITR, CD30, CD40, BAFFR, HVEM, CD7, NKG2C, SLAMF7, NKp80, CD160, B7-H3, or CD83 ligand, or any combination thereof.

8. The combination for use of any of claim 1-2 or 6-7, or the method of any of claims 3-7, wherein the combination of the immunomodulator and the second therapeutic agent is administered together in a single composition or administered separately in two or more different compositions or dosage forms.

9. The combination for use of any of claim 1-2 or 6-8, or the method of any of claims 3-8, wherein the combination of the immunomodulator and the second agent is administered or contacted concurrently with, prior to, or subsequent to, the second agent.

10. The combination for use of any of claim 1-2 or 6-9, or the method of any of claims 3-9, wherein the inhibitor of the immune checkpoint molecule is a soluble ligand or an antibody or antigen-binding fragment thereof, that binds to the immune checkpoint molecule.

11. The combination for use or the method of claim 10, wherein the antibody or antigen-binding fragment thereof is from an IgG1 or IgG4, or an altered form thereof.

12. The combination for use or the method of claim 11, wherein the altered constant region is mutated, to increase or decrease one or more of: Fc receptor binding, antibody glycosylation, the number of cysteine residues, effector cell function, or complement function.

13. The combination of use or the method of claim 10, wherein the antibody molecule is a bispecific or multispecific antibody molecule that has a first binding specificity to PD-1 or PD-L1 and a second binding specifity to TIM-3, CEACAM-1, CEACAM-3, CEACAM-5, LAG-3, or PD-L2.

14. The combination for use of any of claim 1-2 or 6-13, or the method of any of claims 1-13, wherein the immunomodulator is an anti-PD-1 antibody chosen from Nivolumab, Pembrolizumab or Pidilizumab.

15. The combination for use of any of claim 1-2 or 6-13, or the method of any of claims 1-13, wherein the immunomodulator is an anti-PD-L1 antibody chosen from YW243.55.S70, MPDL3280A, MEDI-4736, MSB-0010718C, or MDX-1105.

16. The combination for use of any of claim 1-2 or 6-13, or the method of any of claims 1-13, wherein the immunomodulator is an anti-LAG-3 antibody molecule.

17. The combination for use or the method of claim 16, wherein the anti-LAG-3 antibody molecule is BMS-986016.

18. The combination for use of any of claim 1-2 or 6-13, or the method of any of claims 3-13, wherein the immunomodulator is an anti-PD-1 antibody comprising the heavy chain amino acid sequence of SEQ ID NO: 2 and the light chain amino acid sequence of SEQ ID NO: 3; or the heavy chain amino acid sequence of SEQ ID NO: 4 and the light chain amino acid sequence of SEQ ID NO: 5.

19. The combination for use of any of claim 1-2 or 6-13, or the method of any of claims 3-13, wherein the immunomodulator is the anti-PD-L1 antibody comprising the heavy chain variable amino acid sequence of SEQ ID NO: 6 and the light chain variable amino acid sequence of SEQ ID NO: 7.

20. The combination for use of any of claim 1-2 or 6-13, or the method of any of claims 3-13, wherein the immunomodulator is a TIM-3 inhibitor.

21. The combination for use or the method of claim 20, wherein the TIM-3 inhibitor is an antibody molecule to TIM-3.

22. The combination for use of any of claim 1-2 or 6-21, or the method of any of claims 1-21, wherein the cancer is a solid tumor, a soft tissue tumor chosen from a hematological cancer, leukemia, lymphoma, or myeloma, and a metastatic lesion of any of the aforesaid cancers.

23. The combination for use of any of claim 1-2 or 6-21, or the method of any of claims 1-21, wherein the cancer is a solid tumor from the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), pancreas, colon, rectum, renal-cell carcinoma, liver, lung, non-small cell lung cancer, small intestine or the esophagus.

24. The combination for use of any of claim 1-2 or 6-21, or the method of any of claims 1-21, wherein the cancer is a hematological cancer (e.g., chosen from a Hogdkin's lymphoma, a non-Hodgkin's lymphoma, a lymphocytic leukemia, or a myeloid leukemia).

25. The combination for use of any of claim 1-2 or 6-21, or the method of any of claims 1-21, wherein the cancer is chosen from a cancer disclosed in a publication listed in Table 1.

26. The combination for use of any of claim 1-2 or 6-25, or the method of any of claims 1-25, wherein the subject is a human (e.g., a patient having, or at risk of having, a cancer described herein.

27. The combination for use of any of claim 1-2 or 6-26, or the method of any of claims 1-26, wherein the immunomodulator is an anti-PD-1 antibody molecule administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg., e.g., once a week to once every 2, 3, or 4 weeks.

28. The combination for use or the method of claim 27, wherein the anti-PD-1 antibody molecule is administered at a dose from about 10 to 20 mg/kg every other week.

29. The combination for use or the method of claim 26, wherein the anti-PD-1 antibody molecule, e.g., Nivolumab, is administered intravenously at a dose from about 1 mg/kg to 3 mg/kg, e.g., about 1 mg/kg, 2 mg/kg or 3 mg/kg, every two weeks.

30. The combination for use or the method of claim 26, wherein the anti-PD-1 antibody molecule, e.g., Nivolumab, is administered intravenously at a dose of about 2 mg/kg at 3-week intervals.

31. The combination for use of any of claim 1-2 or 6-30, or the method of any of claims 1-30, wherein the immunomodulator Nivolumab, Pembrolizumab, or MSB0010718C is used in combination with an PKC inhibitor.

32. The combination for use of any of claim 1-2 or 6-30, or the method of any of claims 1-30, wherein the immunomodulator is Nivolumab, Pembrolizumab, or MSB0010718C used in combination with Compound A to treat a disorder selected from, e.g., a cancer, a melanoma, a non-Hodgkin's lymphoma, an inflammatory bowel disease, transplant rejection, an ophthalmic disorder, or psoriasis, wherein Compound A is 3-(1H-indol-3-yl)-4-[2-(4-methyl-1-piperazinyl)-4-quinazolinyl]-1H-pyrrole-2,5-dione.

33. The combination for use or the method of claim 32, wherein Compound A is administered at a dose of about 20 to 600 mg, e.g., about 200 to about 600 mg, e.g., about 50 mg to about 450 mg, about 100 mg to 400 mg, about 150 mg to 350 mg, or about 200 mg to 300 mg, e.g., about 50 mg, 100 mg, 150 mg, 200 mg, 300 mg, 400 mg; 500 mg, or 600 mg every other day, daily, twice or three times a day.

34. The combination for use of any of claim 1-2 or 6-30, or the method of any of claims 1-30, wherein the immunomodulator is Nivolumab, Pembrolizumab, or MSB0010718C used in combination with an HSP90 inhibitor.

35. The combination for use of any of claim 1-2 or 6-30, or the method of any of claims 1-30, wherein the immunomodulator is administered in combination with Compound B to treat a disorder selected from, e.g., a cancer, a multiple myeloma, a non-small cell lung cancer, a lymphoma, a gastric cancer, a breast cancer, a digestive/gastrointestinal cancer, a pancreatic cancer, a colorectal cancer, a solid tumor, or a hematopoiesis disorder, wherein Compound B is 5-(2,4-dihydroxy-5-isopropylphenyl)-N-ethyl-4-(4-(morpholinomethyl)phenyl)isoxazole-3-carboxamide.

36. The combination for use of any of claim 1-2 or 6-30, or the method of any of claims 1-30, wherein the immunomodulator is used in combination with an inhibitor of PI3K and/or mTOR.

37. The combination for use of any of claim 1-2 or 6-30, or the method of any of claims 1-30, wherein the immunomodulator is administered in combination with Compound C to treat a disorder selected from, e.g., a cancer, a prostate cancer, a leukemia (e.g., a lymphocytic leukemia), a breast cancer, a brain cancer, a bladder cancer, a pancreatic cancer, a renal cancer, a solid tumor, or a liver cancer, wherein Compound C is 2-methyl-2-(4-(3-methyl-2-oxo-8-(quinolin-3-yl)-2,3-dihydro-1H-imidazo[4,5-c]quinolin-1-yl)phenyl)propanenitrile.

38. The combination for use of any of claim 1-2 or 6-30, or the method of any of claims 1-30, wherein the immunomodulator is administered in combination with Compound G to treat a disorder selected from, e.g., a cancer or an advanced solid tumor, wherein Compound G is(4S,5R)-3-(2′-amino-2-morpholino-4′-(trifluoromethyl)-[4,5′-bipyrimidin]-6-yl)-4-(hydroxymethyl)-5-methyloxazolidin-2-one.

39. The combination for use of any of claim 1-2 or 6-30, or the method of any of claims 1-30, wherein the immunomodulator is administered in combination with Compound V to treat a disorder selected from, e.g., a cancer, a prostate cancer, a leukemia (e.g., a lymphocytic leukemia), a breast cancer, a brain cancer, a bladder cancer, a pancreatic cancer, a renal cancer, a solid tumor, or a liver cancer, wherein Compound V is 8-(6-Methoxy-pyridin-3-yl)-3-methyl-1-(4-piperazin-1-yl-3-trifluoromethyl-phenyl)-1,3-dihydro-imidazo[4,5-c]quinolin-2-one.

40. The combination for use of any of claim 1-2 or 6-30, or the method of any of claims 1-30, wherein the immunomodulator is used in combination with an inhibitor of cytochrome P450.

41. The combination for use of any of claim 1-2 or 6-30, or the method of any of claims 1-30, wherein the immunomodulator is administered in combination with Compound D to treat a disorder selected from, e.g., a cancer or a prostate cancer.

42. The combination for use of any of claim 1-2 or 6-30, or the method of any of claims 1-30, wherein the immunomodulator is used in combination with an iron chelating agent.

43. The combination for use of any of claim 1-2 or 6-30, or the method of any of claims 1-30, wherein the immunomodulator is administered in combination with Compound E to treat a disorder selected from, e.g., iron overload, hemochromatosis, or myelodysplasia, wherein Compound E is4-[3,5-bis(2-hydroxyphenyl)-1H-1,2,4-triazol-1-yl]-benzoic acid.

44. The combination for use of any of claim 1-2 or 6-30, or the method of any of claims 1-30, wherein the immunomodulator is used in combination with an aromatase inhibitor.

45. The combination for use of any of claim 1-2 or 6-30, or the method of any of claims 1-30, wherein the immunomodulator is administered in combination with Compound F to treat a disorder selected from, e.g., a cancer, a leiomyosarcoma, an endometrium cancer, a breast cancer, a female reproductive system cancer, or a hormone deficiency, wherein Compound F is4,4′-(1H-1,2,4-triazol-1-ylmethylene)bis-benzonitrile.

46. The combination for use of any of claim 1-2 or 6-30, or the method of any of claims 1-30, wherein the immunomodulator is used in combination with an inhibitor of p53.

47. The combination for use of any of claim 1-2 or 6-30, or the method of any of claims 1-30, wherein the immunomodulator is administered in combination with Compound H to treat a disorder selected from, e.g., a cancer or a soft tissue sarcoma, wherein Compound H is (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one.

48. The combination for use of any of claim 1-2 or 6-30, or the method of any of claims 1-30, wherein the immunomodulator is used in combination with an apoptosis inducer.

49. The combination for use of any of claim 1-2 or 6-30, or the method of any of claims 1-30, wherein the immunomodulator is administered in combination with Compound I to treat a disorder selected from, e.g., a cancer, a multiple myeloma, a prostate cancer, a non-small cell lung cancer, a lymphoma, a gastric cancer, a melanoma, a breast cancer, a pancreatic cancer, a digestive/gastroinitestinal cancer, a colorectal cancer, glioblastoma multiforme, a liver cancer, a head and neck cancer, asthma, multiple scelerosis, allergy, Alzheimer's dementia, amyotrophic lateral sclerosis, or rheumatoid arthritis, wherein Compound I is4-[(4-methyl-1-piperazinyl)methyl]-N-[4-methyl-3-[[4-(3-pyridinyl)-2-pyrimidinyl]amino]phenyl]-methanesulfonate-benzamide.

50. The combination for use of claim 49, or the method of claim 49, wherein Compound I is administered at a dose of about 100 to 1000 mg, e.g., about 200 mg to 800 mg, about 300 mg to 700 mg, or about 400 mg to 600 mg, e.g., about 200 mg, 300 mg, 400 mg, 500 mg, 600 mg, or 700 mg; every other day, daily, twice or three times a day.

51. The combination for use of claim 49, or the method of claim 49, wherein Compound I is administered at an oral dose from about 100 mg to 600 mg daily, e.g., about 100 mg, 200 mg, 260 mg, 300 mg, 400 mg, or 600 mg daily.

52. The combination for use of any of claim 1-2 or 6-30, or the method of any of claims 1-30, wherein the immunomodulator is used in combination with an inhibitor of one or more of cytochrome P450, aldosterone or angiogenesis.

53. The combination for use of any of claim 1-2 or 6-30, or the method of any of claims 1-30, wherein the immunomodulator is administered in combination with Compound J to treat a disorder selected from, e.g., Cushing's syndrome, hypertension or heart failure therapy, wherein Compound J is4-[(5)-6,7-dihydro-5H-pyrrolo[1,2-c]imidazol-5-yl]-3-fluorobenzonitrile.

54. The combination for use of any of claim 1-2 or 6-30, or the method of any of claims 1-30, wherein the immunomodulator is used in combination with an SMO receptor inhibitor.

55. The combination for use of any of claim 1-2 or 6-30, or the method of any of claims 1-30, wherein the immunomodulator is administered in combination with Compound K to treat a disorder selected from, e.g., a cancer, a medulloblastoma, a small cell lung cancer, a prostate cancer, a basal cell carcinoma, a pancreatic cancer, or inflammation, wherein Compound K is N-[6-[(2R,6S)-2,6-dimethyl-4-morpholinyl]-3-pyridinyl]-2-methyl-4′-(trifluoromethoxy)-[1,1′-biphenyl]-3-carboxamide, diphosphate.

56. The combination for use of claim 55, or the method of claim 52, wherein Compound K is administered at a dose of about 20 to 500 mg, e.g., about 40 mg to 400 mg, about 50 mg to 300 mg, or about 100 mg to 200 mg, e.g., about 50 mg, 100 mg, 150 mg, 200 mg, 250 mg, or 300 mg; every other day, daily, twice or three times a day.

57. The combination for use of any of claim 1-2 or 6-30, or the method of any of claims 1-30, wherein the immunomodulator is administered in combination with Compound L to treat a disorder selected from, e.g., a cancer, a medulloblastoma, a small cell lung cancer, a prostate cancer, a basal cell carcinoma, a pancreatic cancer, or inflammation, wherein Compound L is (R)-2-(5-(4-(6-benzyl-4,5-dimethylpyridazin-3-yl)-2-methylpiperazin-1-yl)pyrazin-2-yl)propan-2-ol.

58. The combination for use of any of claim 1-2 or 6-30, or the method of any of claims 1-30, wherein the immunomodulator is used in combination with a PRLR inhibitor.

59. The combination for use of any of claim 1-2 or 6-30, or the method of any of claims 1-30, wherein the immunomodulator is administered in combination with Compound M to treat a disorder selected from, e.g., a cancer, a prostate cancer or a breast cancer, wherein Compound M is an isolated antibody that binds the extracellular domain of PRLR comprising a variable light chain amino acid sequence SEQ ID NO: 88 of U.S. Pat. No. 7,867,493, and a variable heavy chain amino acid sequence of SEQ ID NO: 90 of U.S. Pat. No. 7,867,493.

60. The combination for use of any of claim 1-2 or 6-30, or the method of any of claims 1-30, wherein the immunomodulator is used in combination with a Wnt signaling inhibitor.

61. The combination for use of any of claim 1-2 or 6-30, or the method of any of claims 1-30, wherein the immunomodulator is administered in combination with Compound N to treat a disorder selected from, e.g., a solid tumor (e.g., a head and neck cancer, a squamous cell carcinoma, a breast cancer, a pancreatic cancer, or a colon cancer), wherein Compound N is2-(2′,3-dimethyl-[2,4′-bipyridin]-5-yl)-N-(5-(pyrazin-2-yl)pyridin-2-yl)acetamide.

62. The combination for use of claim 61, or the method of claim 61, wherein Compound N is administered at a dose of about 1 to 50 mg, e.g., about 2 mg to 45 mg, about 3 mg to 40 mg, about 5 mg to 35 mg, 5 mg to 10 mg, or about 10 mg to 30 mg, e.g., about 2 mg, 5 mg, 10 mg, 20 mg, 30 mg, or 40 mg; every other day, daily, twice or three times a day.

63. The combination for use of any of claim 1-2 or 6-30, or the method of any of claims 1-30, wherein the immunomodulator is used in combination with a CDK4/6 inhibitor.

64. The combination for use of any of claim 1-2 or 6-30, or the method of any of claims 1-30, wherein the immunomodulator is administered in combination with Compound O to treat a disorder selected from, e.g., a cancer, a mantle cell lymphoma, a liposarcoma, a non-small cell lung cancer, a melanoma, a squamous cell esophageal cancer, or a breast cancer, wherein Compound O is 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide.

65. The combination for use of any of claim 1-2 or 6-30, or the method of any of claims 1-30, wherein the immunomodulator is used in combination with an inhibitor of FGFR2 or FGFR4.

66. The combination for use of any of claim 1-2 or 6-30, or the method of any of claims 1-30, wherein the immunomodulator is administered in combination with Compound P to treat a disorder selected from, e.g., a cancer, a gastric cancer, a breast cancer, a rhabdomyosarcoma, a liver cancer, an adrenal cancer, a lung cancer, an esophageal cancer, a colon cancer, or an endometrial cancer, wherein Compound P is mAb 12425.

67. The combination for use of any of claim 1-2 or 6-30, or the method of any of claims 1-30, wherein the immunomodulator is used in combination with an inhibitor of M-CSF.

68. The combination for use of any of claim 1-2 or 6-30, or the method of any of claims 1-30, wherein the immunomodulator is administered in combination with Compound Q to treat a disorder selected from, e.g., a cancer, a prostate cancer, a breast cancer, or pigmented villonodular synovitis (PVNS), wherein Compound Q is a monoclonal antibody or Fab fragment that comprises 1, 2, 3, 4, 5 or 6 CDRs of monoclonal antibody 5H4.

69. The combination for use of claim 68, or the method of claim 68, wherein Compound Q is administered at a dose of about 10 mg/kg.

70. The combination for use of any of claim 1-2 or 6-30, or the method of any of claims 1-30, wherein the immunomodulator is used in combination with an inhibitor of one or more of c-KIT, histamine release, Flt3 or PKC.

71. The combination for use of any of claim 1-2 or 6-30, or the method of any of claims 1-30, wherein the immunomodulator is administered in combination with Compound R to treat a disorder selected from, e.g., a cancer, a colorectal cancer, a myeloid leukemia, myelodysplastic syndrome, an age-related mascular degeration, a diabetic complication, or a dermatologic disorder, wherein Compound R is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-benzamide.

72. The combination for use of any of claim 1-2 or 6-30, or the method of any of claims 1-30, wherein the immunomodulator is used in combination with an inhibitor of one or more of VEGFR-2, PDGFRbeta, c-KIT or Raf kinase C.

73. The combination for use of any of claim 1-2 or 6-30, or the method of any of claims 1-30, wherein the immunomodulator is administered in combination with Compound S to treat a disorder selected from, e.g., a cancer, a melanoma or a solid tumor, wherein Compound S is 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine.

74. The combination for use of any of claim 1-2 or 6-30, or the method of any of claims 1-30, wherein the immunomodulator is used in combination with a somatostatin agonist and/or a growth hormone release inhibitor.

75. The combination for use of any of claim 1-2 or 6-30, or the method of any of claims 1-30, wherein the immunomodulator is administered in combination with Compound T to treat a disorder selected from, e.g., a cancer, e.g., a prostate cancer, an endocrine cancer, a neurologic cancer, a skin cancer (e.g., melanoma), a pancreatic cancer, a liver cancer, Cushing's syndrome, a gastrointestinal disorder, acromegaly, a liver and biliary tract disorder, or liver cirrhosis, wherein Compound T is cyclo((4R)-4-(2-Aminoethylcarbamoyloxy)-L-prolyl-L-phenylglycyl-D-tryptophyl-L-lysyl-4-O-benzyl-L-tyrosyl-L-phenylalanyl.

76. The combination for use of any of claim 1-2 or 6-30, or the method of any of claims 1-30, wherein the immunomodulator is used in combination with a signal transduction modulator and/or angiogenesis inhibitor.

77. The combination for use of any of claim 1-2 or 6-30, or the method of any of claims 1-30, wherein the immunomodulator is administered in combination with Compound U to treat a disorder selected from, e.g., a cancer, a respiratory/thoracic cancer, a multiple myeloma, a prostate cancer, a non-small cell lung cancer, an endocrine cancer, or a neurological genetic disorder, wherein Compound U is 1-amino-5-fluoro-3-[6-(4-methyl-1-piperazinyl)-1H-benzimidazol-2-yl]-2(1H)-quinolinone.

78. The combination for use of any of claim 1-2 or 6-30, or the method of any of claims 1-30, wherein the immunomodulator is used in combination with an ALK inhibitor.

79. The combination for use of any of claim 1-2 or 6-30, or the method of any of claims 1-30, wherein the immunomodulator is administered in combination with Compound W to treat a disorder selected from, e.g., a cancer, an anaplastic large-cell lymphoma (ALCL), a non-small cell lung carcinoma (NSCLC), or a neuroblastoma, wherein Compound W is N⁶-(2-isopropoxy-5-methyl-4-(1-methylpiperidin-4-yl)phenyl)-N⁴-(2-(isopropylsulfonyl)phenyl)-1H-pyrazolo[3,4-d]pyrimidine-4,6-diamine.

80. The combination for use of any of claim 1-2 or 6-30, or the method of any of claims 1-30, wherein the immunomodulator is used in combination with an IGF-1R inhibitor.

81. The combination for use of any of claim 1-2 or 6-30, or the method of any of claims 1-30, wherein the immunomodulator is administered in combination with Compound X to treat a disorder selected from, e.g., a cancer or a sarcoma, wherein Compound X is3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide.

82. The combination for use of any of claim 1-2 or 6-30, or the method of any of claims 1-30, wherein the immunomodulator is administered in combination with Compound Y to treat a disorder selected from, e.g., a cancer or a sarcoma, wherein Compound Y is5-chloro-N²-(2-fluoro-5-methyl-4-(1-(tetrahydro-2H-pyran-4-yl)piperidin-4-yl)phenyl)-N⁴-(5-methyl-1H-pyrazol-3-yl)pyrimidine-2,4-diamine.

83. The combination for use of any of claim 1-2 or 6-30, or the method of any of claims 1-30, wherein the immunomodulator is administered in combination with Compound Z to treat a disorder selected from, e.g., a cancer or a sarcoma, wherein Compound Z is5-chloro-N2-(4-(1-ethylpiperidin-4-yl)-2-fluoro-5-methylphenyl)-N⁴-(5-methyl-1H-pyrazol-3-yl)pyrimidine-2,4-diamine.

84. The combination for use of any of claim 1-2 or 6-30, or the method of any of claims 1-30, wherein the immunomodulator is used in combination with a P-Glycoprotein 1 inhibitor.

85. The combination for use of any of claim 1-2 or 6-30, or the method of any of claims 1-30, wherein the immunomodulator is administered in combination with Compound AA to treat a disorder selected from, e.g., a cancer or a drug-resistant tumor, wherein Compound AA is 6-[(2S,4R,6E)-4-Methyl-2-(methylamino)-3-oxo-6-octenoic acid]cyclosporin D.

86. The combination for use of any of claim 1-2 or 6-30, or the method of any of claims 1-30, wherein the immunomodulator is used in combination with a VEGFR inhibitor.

87. The combination for use of any of claim 1-2 or 6-30, or the method of any of claims 1-30, wherein the immunomodulator is administered in combination with Compound BB to treat a disorder selected from, e.g., a cancer, wherein Compound BB is N-(4-Chlorophenyl)-4-(4-pyridinylmethyl)-1-phthalazinamine succinate.

88. The combination for use of any of claim 1-2 or 6-30, or the method of any of claims 1-30, wherein the immunomodulator is used in combination with an IDH1 inhibitor.

89. The combination for use of any of claim 1-2 or 6-30, or the method of any of claims 1-30, wherein the immunomodulator is administered in combination with Compound CC to treat a disorder selected from, e.g., a cancer.

90. The combination for use of any of claim 1-2 or 6-30, or the method of any of claims 1-30, wherein the immunomodulator is used in combination with a BCL-ABL inhibitor.

91. The combination for use of any of claim 1-2 or 6-30, or the method of any of claims 1-30, wherein the immunomodulator is administered in combination with Compound DD to treat a disorder selected from, e.g., a cancer, wherein Compound DD is (R)—N-(4-(chlorodifluoromethoxy)phenyl)-6-(3-hydroxypyrrolidin-1-yl)-5-(1H-pyrazol-5-yl)nicotinamide.

92. The combination for use of any of claim 1-2 or 6-30, or the method of any of claims 1-30, wherein the immunomodulator is used in combination with a c-RAF inhibitor.

93. The combination for use of any of claim 1-2 or 6-30, or the method of any of claims 1-30, wherein the immunomodulator is administered in combination with Compound EE to treat a disorder selected from, e.g., a cancer.

94. The combination for use of any of claim 1-2 or 6-30, or the method of any of claims 1-30, wherein the immunomodulator is used in combination with an ERK1/2 ATP inhibitor.

95. The combination for use of any of claim 1-2 or 6-30, or the method of any of claims 1-30, wherein the immunomodulator is administered in combination with Compound FF to treat a disorder selected from, e.g., a cancer.

96. The combination for use of any of claim 1-2 or 6-30, or the method of any of claims 1-30, wherein the immunomodulator is used in combination with a tyrosine kinase inhibitor.

97. The combination for use of any of claim 1-2 or 6-30, or the method of any of claims 1-30, wherein the immunomodulator is administered in combination with Compound GG to treat a disorder selected from, e.g., a cancer, wherein Compound GG is 4-((2-(((1R,2R)-2-hydroxycyclohexyl)amino)benzo[d]thiazol-6-yl)oxy)-N-methylpicolinamide.

98. A composition (e.g., one or more compositions or dosage forms), comprising an immunomodulator (e.g., one or more of: an activator of a costimulatory molecule or an inhibitor of an immune checkpoint molecule) and a second therapeutic agent, e.g., a second therapeutic agent chosen from one or more of the agents listed in Table 1.
